(19) United States
(12) Patent Application Publication
(10) Pub. No.: US 2022/0363746 A1
(43) Pub. Date: Nov. 17, 2022

(54) METHODS FOR TREATING CORONAVIRUS INFECTION AND RESULTING INFLAMMATION-INDUCED LUNG INJURY

(71) Applicant: HUMANIGEN, INC., Burlingame, CA (US)

(72) Inventors: Cameron DURRANT, Oxford, FL (US); Dale CHAPPELL, Dolores, CO (US)

(73) Assignee: HUMANIGEN, INC., Burlingame, CA (US)

(21) Appl. No.: 17/306,884

(22) Filed: May 3, 2021

Related U.S. Application Data
(63) Continuation-in-part of application No. 17/216,660, filed on Mar. 29, 2021.
Application No. 17/216,660 is a continuation-in-part of application No. PCT/US21/21402, filed on Mar. 8, 2021.
(60) Provisional application No. 63/088,971, filed on Oct. 7, 2020.
(60) Provisional application No. 63/072,716, filed on Aug. 31, 2020.
(60) Provisional application No. 63/027,128, filed on May 19, 2020.
(60) Provisional application No. 62/986,751, filed on Mar. 8, 2020.

(51) Int. Cl. C07K 16/24; A61K 31/675; A61K 39/395; A61K 45/06; A61P 31/14; A61P 37/06

(52) U.S. Cl. CPC C07K 16/243; A61K 31/675; A61K 39/3955; A61K 2039/545; A61P 31/14; A61P 37/06; A61K 45/06

(57) ABSTRACT

The present invention provides methods for treating a subject infected with 2019 coronavirus (SARS-CoV-2) comprising administering to the subject a therapeutically effective amount of a GM-CSF antagonist or a therapeutically effective amount of a GM-CSF antagonist and a second drug, including an anti-viral agent, an anti-SARS-CoV-2 vaccine, and serum containing human polyclonal antibodies to SARS-CoV-2.

### CROSS-REFERENCE TO RELATED APPLICATIONS

This application is a continuation-in-part of U.S. application Ser. No. 17/216,660, filed Mar. 29, 2021, which is a continuation-in-part of PCT International Application No. PCT/US21/21402, filed Mar. 8, 2021, which claims priority to U.S. Provisional Application No. 62/986,751, filed Mar. 8, 2020, 63/027,128, filed May 19, 2020, 63/072,716, filed Aug. 31, 2020 and 63/088,971, filed Oct. 7, 2020, each of which is hereby incorporated by reference in its entirety.

### FIELD OF THE INVENTION

This invention relates to methods for treating a subject infected with 2019 coronavirus (SARS-CoV-2), the method comprising administering to the subject a therapeutically effective amount of a GM-CSF antagonist or a GM-CSF antagonist and a second drug, such as anti-viral agent(s), monoclonal antibodies that target and neutralize SARS-CoV-2, serum containing polyclonal antibodies to SARS-CoV-2 or monoclonal antibodies that target the interleukin 6 receptor.

### BACKGROUND OF THE INVENTION

Coronavirus infections, including SARS-CoV-2 (previously named “2019-nCoV” which causes the disease named “COVID-19”), can lead to significant morbidity and mortality with estimated mortality rates for confirmed cases reported to be in the approximately 2%-4% range. The severe clinical features associated with SARS-CoV-2 and other coronaviruses result from an inflammation-induced lung injury (ARDS) requiring ICU care and mechanical ventilation. The inflammation-induced lung injury is a result of cytokine storm (Cytokine Release Syndrome (CRS)) resulting in a hyper-reactive immune response. The inflammation-induced lung injury is not caused directly by the virus, per se, but is a result of an immune response to the virus and can continue after viral titers start to fall. In order to reduce morbidity and mortality, an intervention needs to prevent, shorten the duration of, or reduce cytokine storm in order to reduce the hyper-reactive immune response.

The SARS-CoV-2 pandemic has infected more than 153 million people worldwide causing severe respiratory illness similar to severe acute respiratory syndrome infection. Viral genome analysis has determined that there may be two strains of coronavirus, an aggressive type, the L-type, and the S-type, which may be less virulent. However, since the difference between the two so-called strains is small, scientists have stated that the two identified strains cannot be considered to be separate strains. Accordingly, there is a critical need for improved compositions and therapeutically effective methods for treating and preventing Coronavirus infections, including SARS-CoV-2.

### SUMMARY OF THE INVENTION

In one aspect, the present invention provides a method for treating a subject having emergent COVID-19-associated hyperinflammation (COV-HI), the method comprising administering to the subject a therapeutically effective amount of a pharmaceutical composition comprising an hGM-CSF antagonist.

In another aspect, the present invention provides a method for treating a subject hospitalized with severe COVID-19 pneumonia and having a baseline level of C-reactive protein (CRP) of less than 150 mg/L, the method comprising administering to the subject a therapeutically effective amount of a pharmaceutical composition comprising an hGM-CSF antagonist.

In one aspect, the present invention provides a method for improving invasive mechanical ventilator-free survival (VFS) of a subject infected with 2019 coronavirus (SARS-CoV-2) and having COVID-19 pneumonia, the method comprising administering to the subject a therapeutically effective amount of a pharmaceutical composition comprising a hGM-CSF antagonist.

In another aspect, the present invention provides a method for reducing a treatment emergent serious adverse event (TESAE) of a subject infected with 2019 coronavirus (SARS-CoV-2) and having COVID-19 pneumonia, the method comprising administering to the subject a therapeutically effective amount of a pharmaceutical composition comprising a hGM-CSF antagonist.

In one aspect, the present invention provides a method for reducing time to clinical improvement or time to recovery of a subject infected with 2019 coronavirus (SARS-CoV-2), the method comprising administering to the subject a pharmaceutical composition comprising a therapeutically effective amount of a GM-CSF antagonist, wherein the time to clinical improvement or time to recovery of the subject is reduced by at least 40% compared to the time to clinical improvement or time to recovery of a control subject treated with standard of care and is not administered a GM-CSF antagonist, wherein the subject and the control subject each have severe or critical COVID-19 pneumonia.

In another aspect, the present invention provides a method for treating a subject infected with 2019 coronavirus (SARS-CoV-2), the method comprising administering to the subject a pharmaceutical composition comprising a therapeutically effective amount of a GM-CSF antagonist, wherein the pharmaceutical composition is administered at a dose of from 1200 mg to 1800 mg over 24 hours.

In another aspect, the present invention provides a method for treating a subject infected with 2019 coronavirus (SARS-CoV-2), the method comprising administering to the subject a pharmaceutical composition comprising a therapeutically effective amount of a GM-CSF antagonist, wherein the pharmaceutical composition is administered at a dose of from 1200 mg to 1800 mg over 24 hours, and a therapeutically effective amount of an anti-viral agent.

In still another aspect, the present invention provides a method for preventing and/or treating inflammation-induced lung injury in a subject in need thereof, the method comprising administering to the subject a pharmaceutical composition comprising a therapeutically effective amount of a GM-CSF antagonist, wherein the pharmaceutical composition is administered at a dose of from 1200 mg to 1800 mg over 24 hours.

In one aspect, the present invention provides a method for preventing and/or treating inflammation-induced lung injury in a subject in need thereof, the method comprising administering to the subject a pharmaceutical composition comprising a therapeutically effective amount of a GM-CSF antagonist, wherein the pharmaceutical composition is administered at a dose of from 1200 mg to 1800 mg over 24 hours, and a therapeutically effective amount of an anti-viral agent.

In another aspect, the present invention provides a method for preventing and/or treating cytokine release syndrome (CRS) and/or toxicity induced by CRS, such as ARDS, myocarditis (including Kawasaki's Disease or Kawasaki Shock Syndrome), Multisystem Inflammatory Syndrome in Children (MIS-C), encephalopathy, and disseminated intravascular coagulation (DIC), in a subject in need thereof, the method comprising administering to the subject a pharmaceutical composition comprising a therapeutically effective amount of a GM-CSF antagonist, wherein the pharmaceutical composition is administered at a dose of from 1200 mg to 1800 mg over 24 hours.

In one aspect, the present invention provides a method for preventing and/or treating cytokine release syndrome (CRS) and/or toxicity induced by CRS, such as ARDS, myocarditis (including Kawasaki's Disease or Kawasaki Shock Syndrome), Multisystem Inflammatory Syndrome in Children (MIS-C), encephalopathy, and disseminated intravascular coagulation (DIC), in a subject in need thereof, the method comprising administering to the subject a pharmaceutical composition comprising a therapeutically effective amount of a GM-CSF antagonist, wherein the pharmaceutical composition is administered at a dose of from 1200 mg to 1800 mg over 24 hours, and a therapeutically effective amount of anti-viral agent.

In another aspect, the present invention provides a method for treating a subject infected with a coronavirus (SARS-CoV-2) comprising administering to the subject a pharmaceutical composition comprising a therapeutically effective amount of a GM-CSF antagonist, wherein the pharmaceutical composition is administered at a dose at a dose of from 1200 mg to 1800 mg over 24 hours, and a therapeutically effective amount of an oxygen transporter.

In yet another aspect, the present invention provides a method for treating and/or preventing inflammation-induced lung injury in a subject infected with a coronavirus (SARS-CoV-2) comprising administering to the subject a pharmaceutical composition comprising a therapeutically effective amount of a GM-CSF antagonist, wherein the pharmaceutical composition is administered at a dose of from 1200 mg to 1800 mg over 24 hours, and a therapeutically effective amount of an oxygen transporter.

In one aspect, the present invention provides a method for predicting and preventing a cytokine release syndrome (CRS) and/or inflammation-induced lung injury (ARDS) in a subject infected with 2019 coronavirus (SARS-CoV-2), the method comprising: a) measuring a level of serum ferritin in a blood sample obtained from the subject, wherein a measured level of the serum ferritin of >300 mcg/L indicates (i) the subject has CRS or is at high risk of developing CRS; and/or (ii) the subject has a severe risk factor for developing ARDS, wherein the severe risk for developing ARDS is a risk that is three times greater than the risk for developing ARDS when a measured level of the serum ferritin is <300 mcg/L in a blood sample obtained from a subject; and b) administering to (i) the subject having CRS or at high risk of developing CRS and/or (ii) the subject having the severe risk factor for developing ARDS a pharmaceutical composition comprising a therapeutically effective amount of a GM-CSF antagonist, wherein the pharmaceutical composition is administered at a dose of from 1200 mg to 1800 mg over 24 hours.

In another aspect, the present invention provides a method for predicting and preventing a cytokine release syndrome (CRS) and/or inflammation-induced lung injury (ARDS) in a subject infected with 2019 coronavirus (SARS-CoV-2), the method comprising: a) measuring a level of oxygen saturation by pulse oximetry (SpO₂) of the subject and/or b) performing a chest x-ray or computed tomography (CT) scan, wherein a measured level of the SpO₂ of ≤94% and/or presence of airspace opacity on chest x-ray or ground-glass opacity on CT scan indicate the subject has COVID-19 pneumonia, and (i) the subject has CRS or is at high risk of developing CRS; and/or (ii) the subject has a severe risk factor for developing ARDS, wherein the subject has CRS or is at high risk of developing CRS, wherein the high risk of developing CRS is a risk that is 2.3 times greater than the risk of developing CRS, when a measured level of the SpO₂ of >94% and/or the subject does not have dyspnea and/or has clear lungs on chest x-ray or on CT scan, and/or the subject has the severe risk for developing ARDS, wherein the severe risk for developing ARDS is 2.3 times greater than the risk for developing ARDS when a measured level of the SpO₂ of >94% and/or the subject does not have dyspnea and/or has clear lungs on chest x-ray or on CT scan; and c) administering to (i) the subject having CRS or at high risk of developing CRS and/or (ii) the subject having the severe risk factor for developing ARDS a pharmaceutical composition comprising a therapeutically effective amount of a GM-CSF antagonist, wherein the pharmaceutical composition is administered at a dose of from 1200 mg to 1800 mg over 24 hours.

In another aspect, the present invention provides a method for treating a subject infected with 2019 coronavirus (SARS-CoV-2), the method comprising administering to the subject a therapeutically effective amount of a GM-CSF antagonist.

In still another aspect, the present invention provides a method for treating a subject infected with 2019 coronavirus (SARS-CoV-2), the method comprising administering to the subject a therapeutically effective amount of a GM-CSF antagonist and a therapeutically effective amount of an anti-viral agent. Combination therapy comprising administering to the subject a therapeutically effective amount of a GM-CSF antagonist further comprises administering a second drug, including one or more anti-viral agent(s), an anti-SARS-CoV-2 vaccine, human immunoglobulin (IVIG), monoclonal neutralizing antibodies, and serum containing human polyclonal antibodies to SARS-CoV-2, and a toll-like receptor (TLR) agonist.

In one aspect, the present invention provides a method for preventing and/or treating inflammation-induced lung injury in a subject in need thereof, the method comprising administering to the subject a therapeutically effective amount of a GM-CSF antagonist.

In a further aspect, the present invention provides a method for preventing and/or treating inflammation-induced lung injury in a subject in need thereof, the method comprising administering to the subject a GM-CSF antagonist and an anti-viral agent.

In one aspect, the present invention provides a method for preventing and/or treating cytokine release syndrome (CRS) and/or toxicity induced by CRS, such as ARDS, myocarditis (including Kawasaki's Disease or Kawasaki Shock Syndrome), Multisystem Inflammatory Syndrome in Children (MIS-C), encephalopathy, and disseminated intravascular coagulation (DIC), in a subject in need thereof, the method comprising administering to the subject a therapeutically effective amount of a GM-CSF antagonist. In specific embodiments, the subject in need of prevention and/or treatment of CRS and/or toxicity induced by CRS is a subject infected with 2019 coronavirus (SARS-CoV-2).

In another aspect, the present invention provides a method for preventing and/or treating cytokine release syndrome (CRS) and/or toxicity induced by CRS, such as ARDS, myocarditis (including Kawasaki's Disease or Kawasaki Shock Syndrome), Multisystem Inflammatory Syndrome in Children (MIS-C), encephalopathy, and disseminated intravascular coagulation (DIC), in a subject in need thereof, the method comprising administering to the subject a therapeutically effective amount of a GM-CSF antagonist and a therapeutically effective amount of an anti-viral agent. In particular embodiments, the subject in need of prevention and/or treatment of CRS and/or toxicity induced by CRS is a subject infected with 2019 coronavirus (SARS-CoV-2).

In another aspect, the present invention provides a method for treating a subject infected with a coronavirus (SARS-CoV-2) comprising administering to the subject a therapeutically effective amount of GM-CSF antagonist and a therapeutically effective amount of an oxygen transporter.

In yet another aspect, the present invention provides a method for treating and/or preventing inflammation-induced lung injury in a subject infected with a coronavirus (SARS-CoV-2) comprising administering to the subject a therapeutically effective amount of a GM-CSF antagonist and a therapeutically effective amount of an oxygen transporter.

In another aspect, the present invention provides a method for reducing time to recovery of a subject infected with 2019 coronavirus (SARS-CoV-2) and alleviating the immune-mediated CRS in the subject, the method comprising administering to the subject a pharmaceutical composition comprising a therapeutically effective amount of a GM-CSF antagonist, wherein the pharmaceutical composition is administered at a dose of from 1200 mg to 1800 mg over 24 hours, wherein the time to recovery of the subject is reduced by at least 33% compared to time to recovery of a second subject administered a therapeutically effective amount of an antiviral agent without administration of a GM-CSF antagonist.

In another aspect, the present invention provides a method for treating a subject infected with 2019 coronavirus (SARS-CoV-2) for a time period beyond an initial acute hyper-inflammatory period, the method comprising administering to the subject a pharmaceutical composition comprising a therapeutically effective amount of a GM-CSF antagonist.

Other features and advantages of this invention will become apparent from the following detailed description, examples, and figures. It should be understood, however, that the detailed description and the specific examples while indicating preferred embodiments are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1 shows the natural course of coronavirus infections (SARS) in a typical three-phase manifestation (WHO figure).

FIG. 2 shows pathogenic Th1 cells and inflammatory monocytes have positive correlation with severe pulmonary syndrome in patients infected with SARS-CoV-2. Pathogenic CD4+Th1 (GM-CSF+INFγ+) cells are activated rapidly to produce GM-CSF and other inflammatory cytokines that expand, recruit, and cause trafficking of inflammatory monocytes (CD14+CD16+ with high expression of IL-6) and their progeny. These activated immune cells may enter the pulmonary circulation in large numbers and play an immune damaging role in severe pulmonary syndrome patients. Monoclonal antibodies that target GM-CSF (or the GM-CSF receptor) or interleukin 6 receptor may prevent, or curb immunopathology caused by SARS-CoV-2.

FIG. 3 shows a proposed mechanism for GM-CSF depletion in COVID-19 associated cytokine storm: administered Lenzilumab will bind to and neutralize GM-CSF, and thereby reduce the number of myeloid cells and decrease or eliminate both the production of cytokines and the cascade that causes non-specific killing of respiratory lining cells and reduces or abolishes the clinical symptoms of SARS-CoV-2 infection in a subject. SARS-CoV-2 infects monocytes/macrophages directly via the ACE-2 receptors and through antibody dependent enhancement. Infection with SARS-CoV-2 induces a T cell response through the activation of ThGM and Th17 cells. GM-CSF production by ThGM cells further stimulates monocytes and initiates an immune hyperinflammatory response. Activated monocytes result in production of myeloid derived cytokines, propagation of cytokine storm, trafficking of blood derived monocytes to the lungs, ARDS, and respiratory failure. GM-CSF activated monocytes induce T cell death and result in lymphopenia and worse clinical outcomes.

FIG. 4 shows COVID19 severity over time during three stages of the disease, Stage I (Early Infection), Stage II (Pulmonary Phase) and Stage III (Hyperinflammatory Phase), as measured by the level of lymphocytes, level of myeloid cells and disease severity, together with the clinical symptoms, Lab findings and therapeutic intervention at each stage.

FIG. 5 shows the cumulative % incidence of clinical two-point improvement in 12 patients after therapeutic administration on a compassionate use (CU) of Lenzilumab versus Remdesivir CU in 19 patients over time in day(s) post therapy.

FIGS. 6A-6D show Lenzilumab treatment results in improved clinical outcomes of patients with severe and critical COVID-19 pneumonia. FIG. 6A shows cumulative percentage of patients with at least 2-point improvement in 8 point clinical endpoint scale (95% Kaplan Meier confidence interval displayed). FIG. 6B shows individual temperature over time post-lenzilumab treatment. FIG. 6C shows the percentage of patients with SpO2/FiO2<315 over time post-lenzilumab treatment (95% Kaplan Meier confidence interval displayed). FIG. 6D shows individual hospitalization and oxygen requirement status.

FIGS. 7A-7E show Lenzilumab treatment results in improved inflammatory cytokines and markers of disease severity in patients with severe and critical COVID-19 pneumonia. FIG. 7A shows individual CRP level over time post-lenzilumab treatment. FIG. 7B shows individual IL-6 levels, on Day −1, Day 0 and Day 3 post-lenzilumab treatment. FIG. 7C shows individual platelet levels on Day −1 and Day 3 post-lenzilumab treatment. FIG. 7D shows individual absolute lymphocyte count on Day −1 and Day 3 post-lenzilumab treatment. FIG. 7E shows inflammatory cytokine levels on Day −1 and Day 2 post-lenzilumab treatment. Lenzilumab treatment results in improved inflammatory cytokines in a patient with severe COVID-19 pneumonia Inflammatory cytokine levels on Day −1 and Day 2 post-lenzilumab treatment (*=p<0.05, **=p<0.01)

FIG. 8 shows a comparison of cumulative percentage (%) of clinical two-point improvement over time from administration at DO of each of Lenzilumab compassionate use (CU), Remdesivir and Lopinavir-Ritonavir CU to D28. The time to clinical two-point improvement was more than 50% faster after treatment with Lenzilumab, which had mean days to discharge of 6.3 days versus mean days to discharge of 13.7 days after treatment with Remdesivir and median days to discharge of 13 days after treatment with Lopinavir-Ritonavir. (Table 6 and additional comparative results of Remdesivir compassionate use (CU) in Table 7)

FIG. 9 shows SpO₂/FiO₂ ratio full over time before and after Lenzilumab administration at DO for the 12 patients treated with Lenz CU in Example 8.

FIG. 10 shows individual temperature over time before and post-lenzilumab administration at DO up to D6 for the 12 patients treated with Lenz CU in Example 8.

FIG. 11 shows absolute lymphocyte counts (×10⁹/mL) before and after Lenzilumab administration at DO for the 12 patients treated with Lenz CU in Example 8.

FIG. 12 shows absolute neutrophil counts (×10⁹/mL) before and after Lenzilumab administration at DO for the 12 patients treated with Lenz CU in Example 8.

FIGS. 13A-13B show clinical outcome measures of patients with severe COVID-19 pneumonia, lenzilumab treated vs. untreated. FIG. 13A shows cumulative percentage of patients with at least a 2-point improvement in the 8-point ordinal clinical endpoint scale estimated by Kaplan-Meier curve and compared by log-rank test. FIG. 13B shows mechanical ventilator-free survival estimated by Kaplan-Meier curve and compared by log-rank test.

FIGS. 14A-14B show measurement of oxygenation status of patients treated with lenzilumab vs. untreated. FIG. 14A shows change in mean SpO2/FiO2 ratio displayed at baseline (DO) through day 14 post therapy and compared by repeated measures ANOVA. FIG. 14B shows percentage of patients with ARDS (defined as SpO2/FiO2<315) and compared by repeated measures ANOVA.

FIGS. 15A-15B show radiographic findings upon initial ED examination. FIG. 15A shows initial chest X-ray on presentation. FIG. 15B shows initial chest CT scan on presentation.

FIGS. 16A-16B show supplemental oxygen requirements and lymphocytes as a percentage of complete blood count (CBC) from presentation to discharge of the patient (see Example 11); arrows indicate date of lenzilumab administration. FIG. 16A shows supplemental oxygen requirements (liter flow per minute) from presentation to discharge. FIG. 16B shows lymphocytes as a percentage of CBC from admission to discharge (normal range is 18-45%).

FIG. 17 shows the Primary Endpoint, Ventilator-Free Survival (mITT Population).

FIG. 18 shows a schematic of the enrollment and randomization, as described in Example 13.

FIGS. 19A-19B show Kaplan Meier plots of survival without ventilation. FIG. 19A shows a plot for mITT population. FIG. 19B shows a plot for mITT population with baseline CRP<150 mg/L.

FIG. 20 shows prediction of survival without ventilation by level of CRP cutoff. The hazard ratio for survival without ventilation was calculated for all patients regardless of age with CRP level below the cutoff value.

FIGS. 21A-21B show change in serum CRP following treatment. FIG. 21A shows CRP in mITT population with baseline with CRP<150 mg/L. FIG. 21B shows CRP in mITT population with baseline CRP ≥150 mg/L (with standard error).

### DETAILED DESCRIPTION OF THE INVENTION

In the following detailed description, numerous specific details are set forth in order to provide a thorough understanding of the invention. The subject matter here may be understood more readily by reference to the following detailed description which forms part of this disclosure. It is to be understood that this invention is not limited to the specific products, methods, conditions or parameters described or shown here, and that the terminology used here is for the purpose of describing certain embodiments by way of example only and is not intended to be limiting of the claimed invention.

Highly pathogenic coronaviruses infect the lower airways in humans and cause pneumonia; severe pneumonia is caused by rapid virus replication, immense inflammatory cell infiltration and elevated pro-inflammatory cytokine and chemokine responses, resulting in acute lung injury and acute respiratory distress syndrome. It is this massive immunological response that plays a key role in the adverse clinical manifestations after a person is infected by Coronavirus, including SARS-CoV-2.

Variants of the coronavirus that causes COVID-19 occur when the virus's gene is mutated. Certain variants of the SARS-CoV-2 that are different from the SARS-CoV-2 version first detected in China have been identified. One highly transmissible SARS-CoV-2 variant, now known as B.1.1.7, was initially identified in southeastern England in September 2020 and accounted for about 60% of new COVID-19 cases in the UK in December 2020. SARS-CoV-2 variant, B.1.1.7, has 17 genetic mutations, eight of which are in the spike protein of the coronavirus. Another variant of SARS-CoV-2, called B.1.351, originally was found in South Africa and may have the ability to re-infect people who have recovered from earlier versions of the SARS-CoV-2 coronavirus. A third extremely infectious SARS-CoV-2 variant, P.1, was first detected in Brazil and data suggest that this variant also is able to reinfect people who survived infections with earlier versions of the SARS-CoV-2 coronavirus.

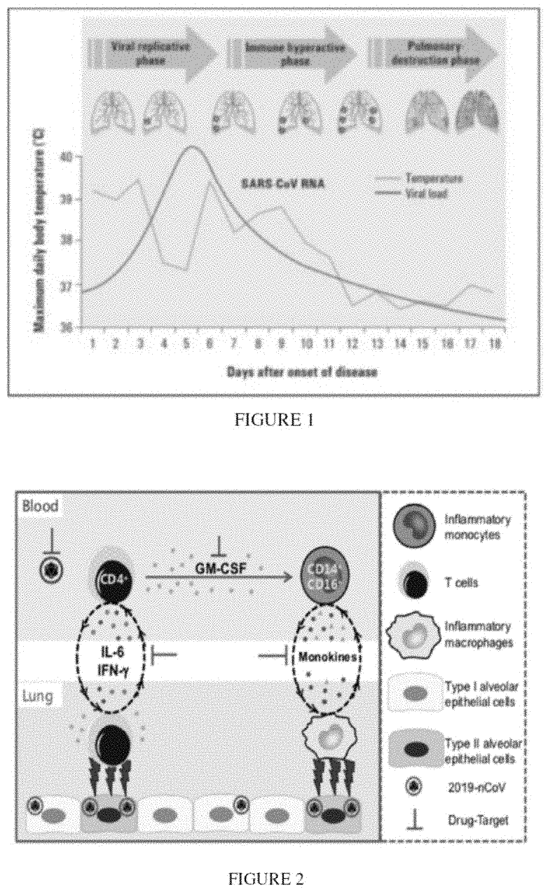

According to the WHO, coronavirus infections are characterized by three phases. Phase 1 is the viral replication phase and last about one week after symptom onset. CT and X-ray show only slowly progressing lung damage. Phase 2 is the immune hyper-reactive phase associated with CRS, with damage caused by the body's immune system, even though viral titers are falling. There is oxygen desaturation, radiological progression of pneumonia and/or development of ARDS. Phase 3 is the pulmonary destruction phase even with low viral titers (FIG. 1).

Activated T cells (including CAR-T cells) produce GM-CSF upon contact with their target. GM-CSF acts as a communication conduit between activated antigen specific T cells/CAR-T cells and the non-specific inflammatory myeloid cell compartment. When T cells become hyper-activated, the resulting GM-CSF over-production causes myeloid cells to expand and traffic to the site of inflammation. These inflammatory myeloid cells then secrete other inflammatory cytokines (IL-1, IL-6, MIP1α, MIP1β, MIG, IP10) and chemokines (MCP-1) that further recruit additional inflammatory myeloid cells resulting in a self-perpetuating inflammatory loop diagnosed clinically as CRS. GM-CSF antagonism, in a xenograft model, has been demonstrated to prevent and/or reduce CRS associated with CAR-T cell therapy by blocking the communication between activated T cells and the inflammatory myeloid cells compartment.

In the case of coronavirus infections including SARS-CoV-2, the activation of virus specific T cells leads to significant GM-CSF production that initiates the CRS process and ultimately leads to inflammation-induced lung injury and, in some cases, death. As is the case with CAR-T induced CRS, using a GM-CSF antagonist can prevent/reduce CRS and the inflammation induced lung injury (FIG. 2).

Since coronavirus has not been shown to infect liver cells, early signs that a patient is developing a CRS related hyper-immune response would be abnormalities in liver enzymes, coagulation markers, albumin, creatinine phosphokinase and lactate dehydrogenase. Elevation of key cytokines/chemokines in the CRS inflammatory cascade such as GM-CSF, MCP-1, IP10, MIP1α, MIP1β, and IL-6 would also be an indication of a hyper-immune response occurring during coronavirus infection. Ferritin is also highly correlated with CRS and can be used as a marker to identify patients at high risk of developing CRS or patients that have already developed CRS. The invention relates to therapeutic compositions comprising an anti-GM-CSF antagonist, as described herein, and to methods for treating a subject infected with 2019 coronavirus (SARS-CoV-2), including but not limited to treatment of infections with highly transmittable SARS-CoV-2 variants B.1.1.7, B.1.351 and P.1, comprising administering anti-GM-CSF antagonists, and/or an anti-GM-CSF antagonist and one or more additional therapeutic agent, including but not limited to anti-viral agents, anti-SARS-CoV-2 vaccines, convalescent plasma, and toll-like receptor (TLR) agonists.

Unless otherwise defined herein, scientific and technical terms used in connection with this application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In this disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, when referring to a measurable value such as an amount, e.g., in mg, a temporal duration, a concentration, and the like, may encompass variations of ±20% or ±10%, more preferably ±5%, even more preferably ±1%, and still more preferably ±0.1% from the specified value, as such variations are appropriate to perform the disclosed methods.

### Clinical Manifestations of COVID-19

The clinical manifestations of COVID-19, the disease caused by severe acute respiratory coronavirus 2 (SARS-CoV-2) infection, range from asymptomatic disease to severe and critical pneumonia. Although viral evasion of host immune response and virus-induced cytopathic effects are believed to be critical for disease progression, most deaths associated with COVID-19 are attributed to the development of immune hyper-response (also known as cytokine release syndrome (CRS) herein) and resultant acute respiratory distress syndrome (ARDS) and multi-organ failure. CRS is characterized by an elevation of inflammatory cytokines resulting in fever, hypotension, capillary leak syndrome, pulmonary edema, disseminated intravascular coagulation, respiratory failure, and ARDS. The development of CRS as a direct result of immune hyper-stimulation has been previously described in patients with autoimmune and lymphoproliferative diseases, as well as in patients with B-cell malignancies receiving chimeric antigen receptor T-cell (CART) therapy, and has been named cytokine release syndrome (CRS). Over the last five years, preclinical studies and correlative science from clinical trials in CART therapy have shed light on the pathophysiology, development, characterization, and management of CRS.

CRS during CART therapy is characterized by activation of myeloid cells and release of inflammatory cytokines and chemokines, including interleukin-6 (IL-6), granulocyte-monocyte colony stimulating factor (GM-CSF), monocyte chemoattractant protein-1 (MCP-1), macrophage inflammatory protein 1α (MIP-1α), Interferon gamma-induced protein 10 (IP-10), and interleukin-1 (IL-1). The cascade, once initiated, can quickly evolve into a cytokine storm, resulting in further activation, expansion and trafficking of myeloid cells, leading to abnormal endothelial activation, increased vascular permeability, and disseminated intravascular coagulation.

### Biomarkers/Inflammatory Markers of CRS in SARS-CoV-2 Infected Patients

The development of immune hyper-response (CRS) in patients with COVID-19 has been associated with elevation of C-reactive protein (CRP), ferritin, and IL-6, as well as correlating with respiratory failure, ARDS, and adverse clinical outcomes. Most significantly, high levels of GM-CSF-secreting Th17 T-cells (Th^{GM} cells) have been associated with disease severity, myeloid cell trafficking to the lungs, and ICU admission. The elevation in inflammatory cytokine levels indicates that post-COVID-19 immune hyperstimulation (CRS) is caused by a similar mechanism, induced by activation of myeloid cells and their trafficking to the lung, resulting in lung injury and ARDS. Tissue CD14+ myeloid cells produce GM-CSF and IL-6, further triggering a cytokine storm cascade. Single-cell RNA sequencing of bronchoalveolar lavage samples from COVID-19 patients with severe ARDS demonstrated an overwhelming infiltration of newly-arrived inflammatory myeloid cells compared to mild COVID-19 disease and healthy controls, consistent with a hyperinflammatory immune (CRS)-mediated pathology.

With this understanding of the pathophysiology of COVID-19, modalities to target inflammatory cytokines and suppress or prevent immune hyperstimulation (CRS) after COVID-19 have been investigated in pilot clinical trials. IL-6 blockade has shown encouraging results. Controlled clinical trials using IL-6 blockade, as well as other immunomodulatory molecules targeting receptor tyrosine kinase are ongoing.

GM-CSF depletion as a strategy to mitigate CRS following CART therapy has developed, as previously described. It has been shown that GM-CSF neutralization results in a reduction in IL-6, MCP-1, MIP-1α, IP-10, vascular endothelial growth factor (VEGF), and tumor necrosis factor-α (TNFα) levels, demonstrating that GM-CSF is an upstream regulator of many inflammatory cytokines that are important in the pathophysiology of CRS. GM-CSF depletion results in modulation of myeloid cell behavior, a specific decrease in their inflammatory cytokines, and a reduction in tissue trafficking, while enhancing T-cell apoptosis machinery. These biological effects prevented both CRS and neuro-inflammation after CART therapy in preclinical models and are being tested in a phase Ib/II clinical trial (NCT 04314843).

### Lenzilumab

Lenzilumab is a first-in-class Humaneered® recombinant monoclonal antibody, derived from mouse antibody LMM102, targeting human GM-CSF, with potential immunomodulatory activity, high binding affinity in the picomolar range, 94% homology to human germline, and has low immunogenicity. Following intravenous administration, lenzilumab binds to and neutralizes GM-CSF, preventing GM-CSF binding to its receptor, thereby preventing GM-CSF-mediated signaling to myeloid progenitor cells. Lenzilumab has been studied across 4 completed clinical trials in healthy volunteers, and persons with asthma, rheumatoid arthritis, and chronic myelomonocytic leukemia. A total of 113 individuals received lenzilumab in these trials; lenzilumab was very well tolerated with a low frequency and severity of adverse events.

In one aspect, the present invention provides a method for treating a subject infected with 2019 coronavirus (SARS-CoV-2), the method comprising administering to the subject a pharmaceutical composition comprising a therapeutically effective amount of a GM-CSF antagonist, wherein the pharmaceutical composition is administered at a dose of from 1200 mg to 1800 mg over 24 hours. In an embodiment, the GM-CSF antagonist is administered at a dose of 400 mg every 8 hours over 24 hours. In another embodiment, the GM-CSF antagonist is administered at a dose of 600 mg every 12 hours over 24 hours. In a particular embodiment, the GM-CSF antagonist is administered at a dose of 600 mg every 8 hours over 24 hours for one day. In a particular embodiment, the GM-CSF antagonist is administered at a dose of 600 mg every 8 hours for a total of three doses over 24 hours. In an embodiment, the administration over 24 hours comprises a total of three doses. In another embodiment, the GM-CSF antagonist is administered at a dose of 800 mg every 12 hours for a total of two doses over 24 hours for one day. In a certain embodiment, the GM-CSF antagonist is administered at a dose of 1800 mg as a single dose for one day. In each of the above-described embodiments, the GM-CSF antagonist is administered intravenously to the subject. In a specific embodiment, the GM-CSF antagonist is neutralizing anti-hGM-CSF antibody Lenzilumab. In an embodiment, the pharmaceutical composition comprises Lenzilumab in a dose of 400 mg. In a particular embodiment, the pharmaceutical composition comprises Lenzilumab in a dose of 600 mg. In another embodiment, the pharmaceutical composition comprises Lenzilumab in a dose of 800 mg. In still another embodiment, the pharmaceutical composition comprises Lenzilumab in a dose of 1800 mg. In the above-described embodiments, the pharmaceutical composition comprising Lenzilumab is administered intravenously to the subject. In another embodiment, the GM-CSF antagonist is chimeric GM-CSF neutralizing antibody KB002 or mouse neutralizing human GM-CSF antibody LMM102. In an embodiment, the pharmaceutical composition comprising a therapeutically effective amount of a GM-CSF antagonist is administered intravenously to the subject. In some embodiments, the GM-CSF antagonist is an anti-GM-SCF antibody selected from the group consisting of Namilumab, Otilimab, Gimsilumab, and TJM2 (TJ003234). In an embodiment, the GM-CSF antagonist is anti-GM-CSF receptor antibody Mavrilimumab. In another embodiment, the method further comprises administering a therapeutically effective amount of an anti-viral agent. In an embodiment, the anti-viral agent is administered to the subject by any suitable route, as described herein. In specific embodiments, the anti-viral agent is administered intravenously to the subject. In another embodiment, the anti-viral agent is administered orally to the subject. In another embodiment, the anti-viral agent is administered by inhalation. In particular embodiments, the anti-viral agent is selected from the group consisting of Aribidol (umifenovir), Favilavir, APN01, defensin mimetic Brilacidin, CCR5 antagonist leronlimab (PRO140), Remdesivir (GS-5734), GS-441524, Galidesivir (BCX4430), Molnupiravir (MK-4482/EIDD-2801), and MK-7110 (CD24Fc) and combinations thereof. In some embodiments, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 (mAbs that target MERS-CoV) or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. Table 1A provides a summary of monoclonal antibody therapies for COVID-19 that are in in clinical trials.

TABLE 1A
mAb-based therapeutics for COVID-19 in clinical trials

| | | Estimated | | |
|---|---|---|---|---|
| | | starting and | | |
| | | primary | | |
| | Trial ID | completion | | |
| Drug code | (Status) | dates | Sponsor | Country |
| REGN-COV2 | NCT04425629 | June 2020-December 2020 | Regeneron/NIAID/ | USA/UK |
| (REGN10933 + | (Phase 2/3) | June 2020-January 2021 | University | |
| REGN10987) | NCT04426695 | July 2020-June 2021 | of Oxford | |
| | (Phase 2/3) | | | |
| | NCT04452318 | | | |
| | (Phase 3) | | | |
| | NCT04381936 | | | |
| | (Phase 3) | | | |
| LY3819253 | NCT04411628 | May 2020-August 2020 | AbCellera/Eli | Canada/USA |
| (LY-CoV555) | (Phase 1) | June 2020-September 2020 | Lilly/NIH | |
| | NCT04427501 | August 2020-March 2021 | | |
| | (Phase 2) | August 2020-July 2021 | | |
| | NCT04497987 | August 2020-November 2020 | | |
| | (Phase 3) | | | |
| | NCT04501978 | | | |
| | (Phase 3) | | | |
| | NCT04518410 | | | |
| | (Phase 2/3) | | | |
| VIR-7831, | NCT04545060 | August 2020-January 2021 | Vir biotechnology/ | USA/UK |
| VIR-7832 | (phase2/3) | | GSK | |
| DXP-593 | NCT04532294; | August 2020-October 2020 | Beigene/Singlomics | China |
| | (Phase 1) | October 2020-February 2021 | Biopharmaceuticals/ | |
| | NCT04551898 | | Peking University etc | |
| | (Phase 2 pending) | | | |
| JS016 | NCT04441918 | June 2020-December 2020 | Junshi Biosciences/ | China/USA |
| | (Phase 1) | | Institute | |
| | | | of Microbiology, | |
| | | | Chinese Academy of | |
| | | | Sciences/Eli Lilly | |
| TY027 | NCT04429529 | June 2020-October 2020 | Tychan | Singapore |
| | (Phase 1) | | | |
| CT-P59 | NCT04525079 | July 2020-November 2020 | Celltrion | South Korea |
| | (Phase 1) | September 2020-December 2020 | | |
| | NCT04593641 | September 2020-December 2020 | | |
| | (Phase 1) | | | |
| | NCT04602000 | | | |
| | (Phase 2/3) | | | |
| BRII-196 | NCT04479631 | July 2020-March 2021 | Brii Bio/TSB | China/USA |
| | (Phase 1) | | Therapeutics/Tsinghua | |
| | | | University | |
| BRII-198 | NCT04479644 | July 2020-March 2021 | Brii Bio/TSB | China/USA |
| | (Phase 1) | | Therapeutics/Tsinghua | |
| | | | University | |
| SCTA01 | NCT04483375 | July 2020-November 2020 | Sinocelltech Ltd/Chinese | China |
| | (Phase 1) | | Academy of Sciences | |
| AZD7442 | NCT04507256 | July 2020-September 2021 | AstraZeneca/Vanderbilt | UK/USA |
| (AZD8895 + | (Phase 1) | | University Medical | |
| AZD1061) | (Phase 3 pending) | | Center/DARPA/BARDA | |
| MW33 | NCT04533048 | July 2020-December 2020 | Mabwell (Shanghai) | China |
| | (Phase 1) | | Bioscience Co., Ltd. | |
| STI- | NCT04454398 | September 2020-February 2021 | Sorrento/Mount Sinai | USA |
| 1499/COVI- | (Phase 1) | | Health System | |
| SHIELD | | | | |
| STI-2020 | NCT04584697 | December 2020-April 2021 | Sorrento/Mount Sinai | USA |
| | (Phase 1/2 | | Health System | |
| | pending) | | | |
| HLX70 | NCT04561076 | December 2020-September 2021 | Hengenix Biotech Inc | USA |
| | (Phase 1) | | | |
| HFB30132A | NCT04590430 | October 2020-July 2021 | HiFiBiO Therapeutics/ABL | USA |
| | (Phase 1) | | bio | |
| ADM03820 | NCT04592549 | November 2020-August 2021 | Ology Bioservices/Enabling | USA |
| | (Phase 1 pending) | | Biotechnologies | |

In an embodiment, the anti-viral agent comprises a combination of antiretroviral drugs, wherein each of the antiretroviral drugs is an inhibitor of HIV-1 protease, or a combination of the inhibitor of HIV-1 protease and a second drug. In another embodiment, the inhibitor of HIV-1 protease is lopinavir or a combination of lopinavir and ritonavir (Lopimune; Aluvia). In still another embodiment, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In another embodiment, the anti-viral agent is SARS-CoV neutralizing antibody CR3022 that binds and neutralizes a receptor binding domain (RBD) of S-protein of SARS-CoV-2. In an embodiment, the method further comprises administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In a particular embodiment, the GM-CSF antagonist is anti-hGM-CSF antibody Lenzilumab. In an embodiment, the herein provided methods further comprise administering to the subject a therapeutically effective amount of a (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In certain embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In another aspect, the present invention provides a method for treating a subject infected with 2019 coronavirus (SARS-CoV-2), the method comprising administering to the subject a pharmaceutical composition comprising a therapeutically effective amount of a GM-CSF antagonist, wherein the pharmaceutical composition is administered at a dose of from 1200 mg to 1800 mg over 24 hours, and a therapeutically effective amount of an anti-viral agent. In an embodiment, the GM-CSF antagonist is administered at a dose of 400 mg every 8 hours over 24 hours. In another embodiment, the GM-CSF antagonist is administered at a dose of 600 mg every 12 hours over 24 hours. In a particular embodiment, the GM-CSF antagonist is administered at a dose of 600 mg every 8 hours over 24 hours for one day. In a particular embodiment, the GM-CSF antagonist is administered at a dose of 600 mg every 8 hours for a total of three doses over 24 hours. In an embodiment, the administration over 24 hours comprises a total of three doses. In another embodiment, the GM-CSF antagonist is administered at a dose of 800 mg every 12 hours for a total of two doses over 24 hours for one day. In a certain embodiment, the GM-CSF antagonist is administered at a dose of 1800 mg as a single dose for one day. In each of the above-described embodiments, the GM-CSF antagonist is administered intravenously to the subject. In a specific embodiment, the GM-CSF antagonist is neutralizing anti-hGM-CSF antibody Lenzilumab. In an embodiment, the pharmaceutical composition comprises Lenzilumab in a dose of 400 mg. In a particular embodiment, the pharmaceutical composition comprises Lenzilumab in a dose of 600 mg. In another embodiment, the pharmaceutical composition comprises Lenzilumab in a dose of 800 mg. In still another embodiment, the pharmaceutical composition comprises Lenzilumab in a dose of 1800 mg. In the above-described embodiments, the pharmaceutical composition comprising Lenzilumab is administered intravenously to the subject. In certain embodiments, the pharmaceutical composition comprising a therapeutically effective amount of a GM-CSF antagonist, e.g., lenzilumab, is administered intravenously to the subject. In another embodiment, the GM-CSF antagonist is chimeric GM-CSF neutralizing antibody KB002 or mouse antibody LMM102. In still another embodiment, the GM-CSF antagonist is an anti-GM-SCF antibody selected from the group consisting of Namilumab, Otilimab, Gimsilumab, and TJM2 (TJ003234). In another embodiment, the GM-CSF antagonist is anti-GM-CSF receptor antibody Mavrilimumab. In an embodiment, the anti-viral agent is administered to the subject by any suitable route, as described herein. In a particular embodiment, the anti-viral agent is administered intravenously to the subject. In another embodiment, the anti-viral agent is administered orally to the subject. In some embodiments, the anti-viral agent is selected from the group consisting of Aribidol (umifenovir), Favilavir, APN01, defensin mimetic Brilacidin, CCR5 antagonist leronlimab (PRO140), Remdesivir (GS-5734), GS-441524, Galidesivir (BCX4430) Molnupiravir (MK-4482/EIDD-2801), and MK-7110 (CD24Fc) and combinations thereof. In various embodiments, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In certain embodiments, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the inhibitor of HIV-1 protease is lopinavir or a combination of lopinavir and ritonavir (Lopimune; Aluvia). In another embodiment, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In yet another embodiment, the anti-viral agent is SARS-CoV neutralizing antibody CR3022 that binds and neutralizes a receptor binding domain (RBD) of S-protein of SARS-CoV-2. In an embodiment of the methods provided herein, the therapeutically effective amount of the GM-CSF antagonist antiviral agent(s), antiretroviral drugs or a combination thereof are administered intravenously to the subject. In an embodiment, the method further comprises administering to the subject a therapeutically effective amount of an anti-SARS-CoV-2 vaccine selected from consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In a particular embodiment, the GM-CSF antagonist is anti-hGM-CSF antibody Lenzilumab. In another embodiment, the herein provided methods further comprise administering to the subject a therapeutically effective amount of (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In an embodiment, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In still another aspect, the present invention provides a method for preventing and/or treating inflammation-induced lung injury in a subject in need thereof, the method comprising administering to the subject a pharmaceutical composition comprising a therapeutically effective amount of a GM-CSF antagonist, wherein the pharmaceutical composition is administered at a dose of from 1200 mg to 1800 mg over 24 hours, and a therapeutically effective amount of an anti-viral agent. In an embodiment, the GM-CSF antagonist is administered at a dose of 400 mg every 8 hours over 24 hours. In another embodiment, the GM-CSF antagonist is administered at a dose of 600 mg every 12 hours over 24 hours. In a particular embodiment, the GM-CSF antagonist is administered at a dose of 600 mg every 8 hours over 24 hours for one day. In a particular embodiment, the GM-CSF antagonist is administered at a dose of 600 mg every 8 hours for a total of three doses over 24 hours. In an embodiment, the administration over 24 hours comprises a total of three doses. In another embodiment, the GM-CSF antagonist is administered at a dose of 800 mg every 12 hours for a total of two doses over 24 hours for one day. In a certain embodiment, the GM-CSF antagonist is administered at a dose of 1800 mg as a single dose for one day. In each of the above-described embodiments, the GM-CSF antagonist is administered intravenously to the subject. In a specific embodiment, the GM-CSF antagonist is neutralizing anti-hGM-CSF antibody Lenzilumab. In an embodiment, the pharmaceutical composition comprises Lenzilumab in a dose of 400 mg. In a particular embodiment, the pharmaceutical composition comprises Lenzilumab in a dose of 600 mg. In another embodiment, the pharmaceutical composition comprises Lenzilumab in a dose of 800 mg. In still another embodiment, the pharmaceutical composition comprises Lenzilumab in a dose of 1800 mg. In the above-described embodiments, the pharmaceutical composition comprising Lenzilumab is administered intravenously to the subject. In a specific embodiment, the GM-CSF antagonist is anti-hGM-CSF antibody Lenzilumab. In an embodiment, the pharmaceutical composition comprising a therapeutically effective amount of a GM-CSF antagonist, e.g., lenzilumab, is administered intravenously to the subject. In an embodiment, the GM-CSF antagonist is chimeric GM-CSF neutralizing antibody KB002 or mouse neutralizing human GM-CSF antibody LMM102. In another embodiment, the GM-CSF antagonist is an anti-GM-SCF antibody selected from the group consisting of Namilumab, Otilimab, Gimsilumab, and TJM2 (TJ003234). In an embodiment, the GM-CSF antagonist is anti-GM-CSF receptor antibody Mavrilimumab. In an embodiment, the anti-viral agent is administered to the subject by any suitable route, as described herein. In a particular embodiment, the anti-viral agent is administered intravenously to the subject. In another embodiment, the anti-viral agent is administered orally to the subject. In an embodiment, the anti-viral agent is selected from the group consisting of Aribidol (umifenovir), Favilavir, APN01, defensin mimetic Brilacidin, CCR5 antagonist leronlimab (PRO140), Remdesivir (GS-5734), GS-441524, Galidesivir (BCX4430) GS-441524, Molnupiravir (MK-4482/EIDD-2801), and MK-7110 (CD24Fc) and combinations thereof. In some embodiments, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In an embodiment, the anti-viral agent comprises a combination of antiretroviral drugs, wherein each of the antiretroviral drugs is an inhibitor of HIV-1 protease, or a combination of the inhibitor of HIV-1 protease and a second drug. In another embodiment, the inhibitor of HIV-1 protease is lopinavir or a combination of lopinavir and ritonavir (Lopimune; Aluvia). In some embodiments, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In an embodiment, the anti-viral agent is SARS-CoV neutralizing antibody CR3022 that binds and neutralizes a receptor binding domain (RBD) of S-protein of SARS-CoV-2. In another embodiment, the methods provided herein further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In a specific embodiment, the GM-CSF antagonist is anti-hGM-CSF antibody Lenzilumab. In another embodiment, the methods provided herein further comprising administering to the subject a therapeutically effective amount of (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In some embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In one aspect, the present invention provides a method for preventing and/or treating inflammation-induced lung injury in a subject in need thereof, the method comprising administering to the subject a pharmaceutical composition comprising a therapeutically effective amount of a GM-CSF antagonist, wherein the pharmaceutical composition is administered at a dose of from 1200 mg to 1800 mg over 24 hours, and a therapeutically effective amount of an anti-viral agent. In an embodiment, the GM-CSF antagonist is administered at a dose of 400 mg every 8 hours over 24 hours. In another embodiment, the GM-CSF antagonist is administered at a dose of 600 mg every 12 hours over 24 hours. In a particular embodiment, the GM-CSF antagonist is administered at a dose of 600 mg every 8 hours over 24 hours for one day. In a particular embodiment, the GM-CSF antagonist is administered at a dose of 600 mg every 8 hours for a total of three doses over 24 hours. In an embodiment, the administration over 24 hours comprises a total of three doses. In another embodiment, the GM-CSF antagonist is administered at a dose of 800 mg every 12 hours for a total of two doses over 24 hours for one day. In a certain embodiment, the GM-CSF antagonist is administered at a dose of 1800 mg as a single dose for one day. In each of the above-described embodiments, the GM-CSF antagonist is administered intravenously to the subject. In a specific embodiment, the GM-CSF antagonist is neutralizing anti-hGM-CSF antibody Lenzilumab. In an embodiment, the pharmaceutical composition comprises Lenzilumab in a dose of 400 mg. In a particular embodiment, the pharmaceutical composition comprises Lenzilumab in a dose of 600 mg. In another embodiment, the pharmaceutical composition comprises Lenzilumab in a dose of 800 mg. In still another embodiment, the pharmaceutical composition comprises Lenzilumab in a dose of 1800 mg. In the above-described embodiments, the pharmaceutical composition comprising Lenzilumab is administered intravenously to the subject. In an embodiment, the anti-viral agent is administered to the subject by any suitable route, as described herein. In a particular embodiment, the anti-viral agent is administered intravenously to the subject. In another embodiment, the anti-viral agent is administered orally to the subject. In a specific embodiment, the GM-CSF antagonist is anti-hGM-CSF antibody Lenzilumab. In an embodiment, the pharmaceutical composition comprising a therapeutically effective amount of a GM-CSF antagonist, e.g., lenzilumab, is administered intravenously to the subject. In another embodiment, the GM-CSF antagonist is chimeric GM-CSF neutralizing antibody KB002 or mouse neutralizing human GM-CSF antibody LMM102. In a further embodiment, the GM-CSF antagonist is an anti-GM-SCF antibody selected from the group consisting of Namilumab, Otilimab, Gimsilumab, and TJM2 (TJ003234). In another embodiment, the GM-CSF antagonist is anti-GM-CSF receptor antibody Mavrilimumab. In some embodiments, the anti-viral agent is selected from the group consisting of Aribidol (umifenovir), Favilavir, APN01, defensin mimetic Brilacidin, CCR5 antagonist leronlimab (PRO140), Remdesivir (GS-5734), GS-441524, Galidesivir (BCX4430) GS-441524, Molnupiravir (MK-4482/EIDD-2801), and MK-7110 (CD24Fc) and combinations thereof. In another embodiment, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In still another embodiment, the anti-viral agent comprises a combination of antiretroviral drugs, wherein each of the antiretroviral drugs is an inhibitor of HIV-1 protease, or a combination of the inhibitor of HIV-1 protease and a second drug. In another embodiment, the inhibitor of HIV-1 protease is lopinavir or a combination of lopinavir and ritonavir (Lopimune; Aluvia). In an embodiment, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In another embodiment, the anti-viral agent is SARS-CoV neutralizing antibody CR3022 that binds and neutralizes a receptor binding domain (RBD) of S-protein of SARS-CoV-2. In some embodiments, the herein provided methods further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In a particular embodiment, the GM-CSF antagonist is anti-hGM-CSF antibody Lenzilumab. In some embodiments, the methods provided herein further comprising administering to the subject a therapeutically effective amount of (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In an embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In one aspect, the present invention provides a method for preventing and/or treating cytokine release syndrome (CRS) and/or toxicity induced by CRS in a subject in need thereof, the method comprising administering to the subject a pharmaceutical composition comprising a therapeutically effective amount of a GM-CSF antagonist, wherein the pharmaceutical composition is administered at a dose of from 1200 mg to 1800 mg over 24 hours. In an embodiment, the toxicity induced by CRS includes, but is not limited to ARDS, myocarditis (including Kawasaki's Disease or Kawasaki Shock Syndrome), Multisystem Inflammatory Syndrome in Children (MIS-C), encephalopathy, and disseminated intravascular coagulation (DIC). In a specific embodiment, the GM-CSF antagonist is neutralizing anti-hGM-CSF antibody Lenzilumab. In an embodiment, the GM-CSF antagonist is administered at a dose of 400 mg every 8 hours over 24 hours. In another embodiment, the GM-CSF antagonist is administered at a dose of 600 mg every 12 hours over 24 hours. In a particular embodiment, the GM-CSF antagonist is administered at a dose of 600 mg every 8 hours over 24 hours for one day. In an embodiment, the administration over 24 hours comprises a total of three doses. In another embodiment, the GM-CSF antagonist is administered at a dose of 800 mg every 12 hours for a total of two doses over 24 hours for one day. In a certain embodiment, the GM-CSF antagonist is administered at a dose of 1800 mg as a single dose for one day. In each of the above-described embodiments, the GM-CSF antagonist is administered intravenously to the subject. In a specific embodiment, the GM-CSF antagonist is neutralizing anti-hGM-CSF antibody Lenzilumab. In a particular embodiment, the pharmaceutical composition comprising the therapeutically effective amount of the GM-CSF antagonist, e.g., lenzilumab, is administered intravenously to the subject. In an embodiment, the pharmaceutical composition comprises Lenzilumab in a dose of 400 mg. In a particular embodiment, the pharmaceutical composition comprises Lenzilumab in a dose of 600 mg. In another embodiment, the pharmaceutical composition comprises Lenzilumab in a dose of 800 mg. In still another embodiment, the pharmaceutical composition comprises Lenzilumab in a dose of 1800 mg. In the above-described embodiments, the pharmaceutical composition comprising Lenzilumab is administered intravenously to the subject. In another embodiment, the GM-CSF antagonist is chimeric GM-CSF neutralizing antibody KB002 or mouse neutralizing human GM-CSF antibody LMM102. In yet another embodiment, the GM-CSF antagonist is an anti-GM-SCF antibody selected from the group consisting of Namilumab, Otilimab, Gimsilumab, and TJM2 (TJ003234). In still another embodiment, the GM-CSF antagonist is anti-GM-CSF receptor antibody Mavrilimumab. In another embodiment, the methods provided herein further comprise administering a therapeutically effective amount of an anti-viral agent. In an embodiment, the anti-viral agent is administered to the subject by any suitable route, as described herein. In specific embodiments, the anti-viral agent is administered intravenously to the subject. In another embodiment, the anti-viral agent is administered orally to the subject. In an embodiment, the anti-viral agent is selected from the group consisting of Aribidol (umifenovir), Favilavir, APN01, defensin mimetic Brilacidin, CCR5 antagonist leronlimab (PRO140), Remdesivir (GS-5734), GS-441524, Galidesivir (BCX4430) GS-441524, Molnupiravir (MK-4482/EIDD-2801), and MK-7110 (CD24Fc) and combinations thereof. In another embodiment, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In some embodiments, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the inhibitor of HIV-1 protease is lopinavir or a combination of lopinavir and ritonavir (Lopimune; Aluvia). In another embodiment, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In another embodiment, the anti-viral agent is SARS-CoV neutralizing antibody CR3022 that binds and neutralizes a receptor binding domain (RBD) of S-protein of SARS-CoV-2. In a further embodiment, the methods provided herein further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In a particular embodiment, the GM-CSF antagonist is anti-hGM-CSF antibody Lenzilumab. In another embodiment, the herein provided methods further comprise administering to the subject a therapeutically effective amount of (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In some embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In one aspect, the present invention provides a method for preventing and/or treating cytokine release syndrome (CRS) and/or toxicity induced by CRS in a subject in need thereof, the method comprising administering to the subject a pharmaceutical composition comprising a therapeutically effective amount of a GM-CSF antagonist, wherein the pharmaceutical composition is administered at a dose of from 1200 mg to 1800 mg over 24 hours, and a therapeutically effective amount of anti-viral agent. In a specific embodiment, the GM-CSF antagonist is neutralizing anti-hGM-CSF antibody Lenzilumab. In an embodiment, the GM-CSF antagonist is administered at a dose of 400 mg every 8 hours over 24 hours. In another embodiment, the GM-CSF antagonist is administered at a dose of 600 mg every 12 hours over 24 hours. In a particular embodiment, the GM-CSF antagonist is administered at a dose of 600 mg every 8 hours over 24 hours for one day. In an embodiment, the administration over 24 hours comprises a total of three doses. In another embodiment, the GM-CSF antagonist is administered at a dose of 800 mg every 12 hours for a total of two doses over 24 hours for one day. In a certain embodiment, the GM-CSF antagonist is administered at a dose of 1800 mg as a single dose for one day. In each of the above-described embodiments, the pharmaceutical composition comprising the therapeutically effective amount of the GM-CSF antagonist, e.g., lenzilumab, is administered intravenously to the subject. In an embodiment, the pharmaceutical composition comprises Lenzilumab in a dose of 400 mg. In a particular embodiment, the pharmaceutical composition comprises Lenzilumab in a dose of 600 mg. In another embodiment, the pharmaceutical composition comprises Lenzilumab in a dose of 800 mg. In still another embodiment, the pharmaceutical composition comprises Lenzilumab in a dose of 1800 mg. In the above-described embodiments, the pharmaceutical composition comprising Lenzilumab is administered intravenously to the subject. In specific embodiments, the subject in need of prevention and/or treatment of CRS and/or toxicity induced by CRS is a subject infected with 2019 coronavirus (SARS-CoV-2). In an embodiment, the toxicity induced by CRS includes, but is not limited to ARDS, myocarditis (including Kawasaki's Disease or Kawasaki Shock Syndrome), Multisystem Inflammatory Syndrome in Children (MIS-C), encephalopathy, and disseminated intravascular coagulation (DIC). In a particular embodiment, the GM-CSF antagonist is anti-hGM-CSF antibody Lenzilumab. In another embodiment, the GM-CSF antagonist is chimeric GM-CSF neutralizing antibody KB002 or mouse neutralizing human GM-CSF antibody LMM102. In some embodiments, the GM-CSF antagonist is an anti-GM-SCF antibody selected from the group consisting of Namilumab, Otilimab, Gimsilumab, and TJM2 (TJ003234). In another embodiment, the GM-CSF antagonist is anti-GM-CSF receptor antibody Mavrilimumab. In various embodiments, the anti-viral agent is selected from the group consisting of Aribidol (umifenovir), Favilavir, APN01, defensin mimetic Brilacidin, CCR5 antagonist leronlimab (PRO140), Remdesivir (GS-5734), GS-441524, Galidesivir (BCX4430), Molnupiravir (MK-4482/EIDD-2801), and MK-7110 (CD24Fc) and combinations thereof. In some embodiments, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In certain embodiments, the anti-viral agent is administered to the subject by any suitable route, as described herein. In specific embodiments, the anti-viral agent is administered intravenously to the subject. In another embodiment, the anti-viral agent is administered orally to the subject. In another embodiment, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the inhibitor of HIV-1 protease is lopinavir or a combination of lopinavir and ritonavir (Lopimune; Aluvia). In another embodiment, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In an embodiment, the anti-viral agent is SARS-CoV neutralizing antibody CR3022 that binds and neutralizes a receptor binding domain (RBD) of S-protein of SARS-CoV-2. In still another embodiment, the methods provided herein further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In a specific embodiment, the GM-CSF antagonist is anti-hGM-CSF antibody Lenzilumab. In yet another embodiment, the methods provided herein of further comprise administering to the subject a therapeutically effective amount of (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In various embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In another aspect, the present invention provides a method for treating a subject infected with a coronavirus (SARS-CoV-2) comprising administering to the subject a pharmaceutical composition comprising a therapeutically effective amount of a GM-CSF antagonist, wherein the pharmaceutical composition is administered at a dose of from 1200 mg to 1800 mg over 24 hours, and a therapeutically effective amount of an oxygen transporter. In a specific embodiment, the GM-CSF antagonist is neutralizing anti-hGM-CSF antibody Lenzilumab. In an embodiment, the GM-CSF antagonist is administered at a dose of 400 mg every 8 hours over 24 hours. In another embodiment, the GM-CSF antagonist is administered at a dose of 600 mg every 12 hours over 24 hours. In a particular embodiment, the GM-CSF antagonist is administered at a dose of 600 mg every 8 hours over 24 hours for one day. In an embodiment, the administration over 24 hours comprises a total of three doses. In another embodiment, the GM-CSF antagonist is administered at a dose of 800 mg every 12 hours for a total of two doses over 24 hours for one day. In a certain embodiment, the GM-CSF antagonist is administered at a dose of 1800 mg as a single dose for one day. In each of the above-described embodiments, the pharmaceutical composition comprising the therapeutically effective amount of the GM-CSF antagonist is administered intravenously to the subject. In an embodiment, the pharmaceutical composition comprises Lenzilumab in a dose of 400 mg. In a particular embodiment, the pharmaceutical composition comprises Lenzilumab in a dose of 600 mg. In another embodiment, the pharmaceutical composition comprises Lenzilumab in a dose of 800 mg. In still another embodiment, the pharmaceutical composition comprises Lenzilumab in a dose of 1800 mg. In the above-described embodiments, the pharmaceutical composition comprising Lenzilumab is administered intravenously to the subject. In an embodiment, the oxygen transporter is BXT25. In a specific embodiment, the GM-CSF antagonist is anti-hGM-CSF antibody Lenzilumab. In another embodiment, the GM-CSF antagonist is chimeric GM-CSF neutralizing antibody KB002 or mouse neutralizing human GM-CSF antibody LMM102. In yet another embodiment, the GM-CSF antagonist is an anti-GM-SCF antibody selected from the group consisting of Namilumab, Otilimab, Gimsilumab, and TJM2 (TJ003234). In another embodiment, the GM-CSF antagonist is anti-GM-CSF receptor antibody Mavrilimumab. In another embodiment, the herein provided method further comprises administering a therapeutically effective amount of an anti-viral agent. In an embodiment, the anti-viral agent is administered to the subject by any suitable route, as described herein. In specific embodiments, the anti-viral agent is administered intravenously to the subject. In another embodiment, the anti-viral agent is administered orally to the subject. In some embodiments, the anti-viral agent is selected from the group consisting of Aribidol (umifenovir), Favilavir, APN01, defensin mimetic Brilacidin, CCR5 antagonist leronlimab (PRO140), Remdesivir (GS-5734), GS-441524, Galidesivir (BCX4430), Molnupiravir (MK-4482/EIDD-2801), and MK-7110 (CD24Fc) and combinations thereof. In certain embodiments, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In an embodiment, the anti-viral agent comprises a combination of antiretroviral drugs, wherein each of the antiretroviral drugs is an inhibitor of HIV-1 protease, or a combination of the inhibitor of HIV-1 protease and a second drug. In another embodiment, the inhibitor of HIV-1 protease is lopinavir or a combination of lopinavir and ritonavir (Lopimune; Aluvia). In still another embodiment, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In another embodiment, the methods provided herein further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In some embodiments, the methods provided herein of further comprise administering to the subject a therapeutically effective amount of (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In an embodiment, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In a further aspect, the present invention provides a method for treating and/or preventing inflammation-induced lung injury in a subject infected with a coronavirus (SARS-CoV-2) comprising administering to the subject a pharmaceutical composition comprising a therapeutically effective amount of a GM-CSF antagonist, wherein the pharmaceutical composition is administered at a dose of from 1200 mg to 1800 mg over 24 hours, and a therapeutically effective amount of an oxygen transporter. In a specific embodiment, the GM-CSF antagonist is neutralizing anti-hGM-CSF antibody Lenzilumab. In an embodiment, the GM-CSF antagonist is administered at a dose of 400 mg every 8 hours over 24 hours. In another embodiment, the GM-CSF antagonist is administered at a dose of 600 mg every 12 hours over 24 hours. In a particular embodiment, the GM-CSF antagonist is administered at a dose of 600 mg every 8 hours over 24 hours for one day. In an embodiment, the administration over 24 hours comprises a total of three doses. In another embodiment, the GM-CSF antagonist is administered at a dose of 800 mg every 12 hours for a total of two doses over 24 hours for one day. In a certain embodiment, the GM-CSF antagonist is administered at a dose of 1800 mg as a single dose for one day. In each of the above-described embodiments, the pharmaceutical composition comprising the therapeutically effective amount of a GM-CSF antagonist, e.g., lenzilumab, is administered intravenously to the subject. In an embodiment, the pharmaceutical composition comprises Lenzilumab in a dose of 400 mg. In a particular embodiment, the pharmaceutical composition comprises Lenzilumab in a dose of 600 mg. In another embodiment, the pharmaceutical composition comprises Lenzilumab in a dose of 800 mg. In still another embodiment, the pharmaceutical composition comprises Lenzilumab in a dose of 1800 mg. In the above-described embodiments, the pharmaceutical composition comprising Lenzilumab is administered intravenously to the subject. In some embodiments, the oxygen transporter is BXT25. In a particular embodiment, the GM-CSF antagonist is anti-hGM-CSF antibody Lenzilumab. In another embodiment, the GM-CSF antagonist is chimeric GM-CSF neutralizing antibody KB002 or mouse neutralizing human GM-CSF antibody LMM102. In some embodiments, the GM-CSF antagonist is an anti-GM-SCF antibody selected from the group consisting of Namilumab, Otilimab, Gimsilumab, and TJM2 (TJ003234). In another embodiment, the GM-CSF antagonist is anti-GM-CSF receptor antibody Mavrilimumab. In another embodiment, the herein provided method further comprises administering a therapeutically effective amount of an anti-viral agent. In an embodiment, the anti-viral agent is administered to the subject by any suitable route, as described herein. In specific embodiments, the anti-viral agent is administered intravenously to the subject. In another embodiment, the anti-viral agent is administered orally to the subject. In yet another embodiment, the anti-viral agent is selected from the group consisting of Aribidol (umifenovir), Favilavir, APN01, defensin mimetic Brilacidin, CCR5 antagonist leronlimab (PRO140), Remdesivir (GS-5734), GS-441524, Galidesivir (BCX4430), Molnupiravir (MK-4482/EIDD-2801), and MK-7110 (CD24Fc) and combinations thereof. In some embodiments, the anti-viral agent comprises a combination of fully human neutralizing monoclonal antibodies (mAb) against S-protein of MERS-CoV, or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In another embodiment, the anti-viral agent comprises a combination of antiretroviral drugs, wherein each of the antiretroviral drugs is an inhibitor of HIV-1 protease, or a combination of the inhibitor of HIV-1 protease and a second drug. In certain embodiments, the inhibitor of HIV-1 protease is lopinavir or a combination of lopinavir and ritonavir (Lopimune; Aluvia). In some embodiments, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In another embodiment, the methods provided herein further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In an embodiment, the methods provided herein of further comprise administering to the subject a therapeutically effective amount of (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In some embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In another aspect, the present invention provides a method for reducing time to recovery of a subject infected with 2019 coronavirus (SARS-CoV-2) and alleviating the immune-mediated CRS in the subject, the method comprising administering to the subject a pharmaceutical composition comprising a therapeutically effective amount of a GM-CSF antagonist, wherein the pharmaceutical composition is administered at a dose of from 1200 mg to 1800 mg over 24 hours, wherein the time to recovery of the subject is reduced by at least 33% compared to time to recovery of a second subject administered a therapeutically effective amount of an antiviral agent without administration of a GM-CSF antagonist. In a particular embodiment, the GM-CSF antagonist is neutralizing anti-hGM-CSF antibody Lenzilumab. In another embodiment, the GM-CSF antagonist is administered at a dose of 400 mg every 8 hours for a total of three doses over 24 hours. In a specific embodiment, the GM-CSF antagonist is administered at a dose of 600 mg every 8 hours for a total of three doses over 24 hours for one day. In an embodiment, the GM-CSF antagonist is administered at a dose of 800 mg every 12 hours for a total of two doses over 24 hours for one day. In another embodiment, the GM-CSF antagonist is administered at a dose of 1800 mg as a single dose for one day. In an embodiment, the GM-CSF antagonist is chimeric GM-CSF neutralizing antibody KB002 or mouse neutralizing human GM-CSF antibody LMM102. In another embodiment, the GM-CSF antagonist is an anti-GM-SCF antibody selected from the group consisting of Namilumab, Otilimab, Gimsilumab, and TJM2 (TJ003234). In still another embodiment, the GM-CSF antagonist is anti-GM-CSF receptor antibody Mavrilimumab. In yet another embodiment, the method for reducing time to recovery of the subject infected with 2019 coronavirus (SARS-CoV-2) and alleviating the immune-mediated CRS in the subject, further comprises administering a therapeutically effective amount of an anti-viral agent. In an embodiment, the anti-viral agent is selected from the group consisting of Aribidol (umifenovir), Favilavir, APN01, defensin mimetic Brilacidin, CCR5 antagonist leronlimab (PRO140), Remdesivir (GS-5734), GS-441524, Galidesivir (BCX4430), Molnupiravir (MK-4482/EIDD-2801), and MK-7110 (CD24Fc) and combinations thereof. In another embodiment, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In an embodiment, the anti-viral agent comprises a combination of antiretroviral drugs, wherein each of the antiretroviral drugs is an inhibitor of HIV-1 protease, or a combination of the inhibitor of HIV-1 protease and a second drug. In another embodiment, the inhibitor of HIV-1 protease is lopinavir or a combination of lopinavir and ritonavir (Lopimune; Aluvia). In still another embodiment, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In some embodiments, the antiviral agent administered to the second subject is selected from the group consisting of Aribidol (umifenovir), Favilavir, APN01, defensin mimetic Brilacidin, CCR5 antagonist leronlimab (PRO140), Remdesivir (GS-5734), GS-441524, Galidesivir (BCX4430), Molnupiravir (MK-4482/EIDD-2801), and MK-7110 (CD24Fc) and combinations thereof. In another embodiment, the antiviral agent administered to the second subject comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In an embodiment, the anti-viral agent comprises a combination of antiretroviral drugs, wherein each of the antiretroviral drugs is an inhibitor of HIV-1 protease, or a combination of the inhibitor of HIV-1 protease and a second drug. In another embodiment, the inhibitor of HIV-1 protease is lopinavir or a combination of lopinavir and ritonavir (Lopimune; Aluvia). In some embodiments, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In a particular embodiment, the GM-CSF antagonist is lenzilumab and the antiviral agent administered to the second subject is Remdesivir (GS-5734), GS-441524, Molnupiravir (MK-4482/EIDD-2801), MK-7110 (CD24Fc) and combinations thereof and wherein the time to recovery of the subject is reduced by at least 50% compared to the time to recovery of the second subject administered the therapeutically effective amount of the antiviral agent without administration of lenzilumab. In a specific embodiment, the GM-CSF antagonist is lenzilumab and the antiviral agent administered to the second subject is a combination of lopinavir and ritonavir (Lopimune; Aluvia), and wherein the time to recovery of the subject is reduced by at least 50% compared to the time to recovery of the second subject administered the therapeutically effective amount of the antiviral agent without administration of lenzilumab. In another embodiment, the methods provided herein further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In some embodiments, the methods provided herein of further comprise administering to the subject a therapeutically effective amount of (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In certain embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject

In still another aspect, the present invention provides a method for predicting and preventing a cytokine release syndrome (CRS) and/or inflammation-induced lung injury (ARDS) in a subject infected with 2019 coronavirus (SARS-CoV-2), the method comprising: a) measuring a level of serum ferritin in a blood sample obtained from the subject, wherein a measured level of the serum ferritin of >300 mcg/L indicates (i) the subject has CRS or is at high risk of developing CRS; and/or (ii) the subject has a severe risk factor for developing ARDS, wherein the severe risk for developing ARDS is a risk that is three times greater than the risk for developing ARDS when a measured level of the serum ferritin is <300 mcg/L in a blood sample obtained from a subject; and b) intravenously administering to (i) the subject having CRS or at high risk of developing CRS and/or (ii) the subject having the severe risk factor for developing ARDS a pharmaceutical composition comprising a therapeutically effective amount of a GM-CSF antagonist, wherein the pharmaceutical composition is administered at a dose of from 1200 mg to 1800 mg over 24 hours. In a specific embodiment, the GM-CSF antagonist is neutralizing anti-hGM-CSF antibody Lenzilumab. In an embodiment, the GM-CSF antagonist is administered at a dose of 400 mg every 8 hours over 24 hours. In another embodiment, the GM-CSF antagonist is administered at a dose of 600 mg every 12 hours over 24 hours. In a particular embodiment, the GM-CSF antagonist is administered at a dose of 600 mg every 8 hours over 24 hours for one day. In an embodiment, the administration over 24 hours comprises a total of three doses. In another embodiment, the GM-CSF antagonist is administered at a dose of 800 mg every 12 hours for a total of two doses over 24 hours for one day. In a certain embodiment, the GM-CSF antagonist is administered at a dose of 1800 mg as a single dose for one day. In each of the above-described embodiments, the GM-CSF antagonist is administered intravenously to the subject. In an embodiment, the pharmaceutical composition comprises Lenzilumab in a dose of 400 mg. In a particular embodiment, the pharmaceutical composition comprises Lenzilumab in a dose of 600 mg. In another embodiment, the pharmaceutical composition comprises Lenzilumab in a dose of 800 mg. In still another embodiment, the pharmaceutical composition comprises Lenzilumab in a dose of 1800 mg. In the above-described embodiments, the pharmaceutical composition comprising Lenzilumab is administered intravenously to the subject. In another embodiment, the herein provided method further comprises administering a therapeutically effective amount of an anti-viral agent. In an embodiment, the anti-viral agent is administered to the subject by any suitable route, as described herein. In specific embodiments, the anti-viral agent is administered intravenously to the subject. In another embodiment, the anti-viral agent is administered orally to the subject. In some embodiments, the anti-viral agent is selected from the group consisting of Aribidol (umifenovir), Favilavir, APN01, defensin mimetic Brilacidin, CCR5 antagonist leronlimab (PRO140), Remdesivir (GS-5734), GS-441524, Galidesivir (BCX4430), Molnupiravir (MK-4482/EIDD-2801), and MK-7110 (CD24Fc) and combinations thereof. In certain embodiments, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In an embodiment, the anti-viral agent comprises a combination of antiretroviral drugs, wherein each of the antiretroviral drugs is an inhibitor of HIV-1 protease, or a combination of the inhibitor of HIV-1 protease and a second drug. In another embodiment, the inhibitor of HIV-1 protease is lopinavir or a combination of lopinavir and ritonavir (Lopimune; Aluvia). In still another embodiment, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In yet another embodiment, the methods provided herein further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In an embodiment, the methods provided herein of further comprise administering to the subject a therapeutically effective amount of (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In various embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In certain embodiments, the TLR7 agonist, TLR8 agonist and/or the TLR7/8 dual agonist is administered to a male subject

In another aspect, the present invention provides a method for predicting and preventing a cytokine release syndrome (CRS) and/or inflammation-induced lung injury (ARDS) in a subject infected with 2019 coronavirus (SARS-CoV-2), the method comprising: a) measuring a level of oxygen saturation by pulse oximetry (SpO₂) of the subject and/or b) performing a chest x-ray or computed tomography (CT) scan, wherein a measured level of the SpO₂ of ≤94% and/or presence of airspace opacity on chest x-ray or ground-glass opacity on CT scan indicate the subject has COVID-19 pneumonia, and (i) the subject has CRS or is at high risk of developing CRS; and/or (ii) the subject has a severe risk factor for developing ARDS, wherein the subject has CRS or is at high risk of developing CRS, wherein the high risk of developing CRS is a risk that is 2.3 times greater than the risk of developing CRS when a measured level of the SpO₂ is >94% and/or the patient does not have dyspnea and/or has clear lungs on chest x-ray or on CT scan, and/or the subject has the severe risk for developing ARDS, wherein the severe risk for developing ARDS is 2.3 times greater than the risk for developing ARDS when a measured level of the SpO₂ is >94% and/or the patient does not have dyspnea and/or has clear lungs on chest x-ray or on CT scan; and c) administering to (i) the subject having CRS or at high risk of developing CRS and/or (ii) the subject having the severe risk factor for developing ARDS a pharmaceutical composition comprising a therapeutically effective amount of a GM-CSF antagonist, wherein the pharmaceutical composition is administered at a dose of from 1200 mg to 1800 mg over 24 hours. In a specific embodiment, the GM-CSF antagonist is neutralizing anti-hGM-CSF antibody Lenzilumab. In an embodiment, the GM-CSF antagonist is administered at a dose of 400 mg every 8 hours over 24 hours. In another embodiment, the GM-CSF antagonist is administered at a dose of 600 mg every 12 hours over 24 hours. In a particular embodiment, the GM-CSF antagonist is administered at a dose of 600 mg every 8 hours over 24 hours for one day. In an embodiment, the administration over 24 hours comprises a total of three doses. In another embodiment, the GM-CSF antagonist is administered at a dose of 800 mg every 12 hours for a total of two doses over 24 hours for one day. In a certain embodiment, the GM-CSF antagonist is administered at a dose of 1800 mg as a single dose for one day. In each of the above-described embodiments, the GM-CSF antagonist is administered intravenously to the subject. In an embodiment, the pharmaceutical composition comprises Lenzilumab in a dose of 400 mg. In a particular embodiment, the pharmaceutical composition comprises Lenzilumab in a dose of 600 mg. In another embodiment, the pharmaceutical composition comprises Lenzilumab in a dose of 800 mg. In still another embodiment, the pharmaceutical composition comprises Lenzilumab in a dose of 1800 mg. In the above-described embodiments, the pharmaceutical composition comprising Lenzilumab is administered intravenously to the subject.

In various embodiments of the therapeutic methods described herein, the GM-CSF antagonist is chimeric GM-CSF neutralizing antibody KB002. In an embodiment, the GM-CSF antagonist is an anti-GM-SCF antibody selected from the group consisting of Namilumab, Otilimab, Gimsilumab, and TJM2 (TJ003234). In another embodiment, the GM-CSF antagonist is anti-GM-CSF receptor antibody Mavrilimumab.

As defined herein a subject is “at high risk for developing CRS” and “at high risk of CRS related inflammatory lung injury” when the person has one or more of the following clinical indicators (also called clinical markers):

Ferritin elevation of >300 mcg/L,
CRP elevation of >8 mg/L
alanine aminotransferase (ALT) elevation that is ten or more times higher than the normal ALT range of 7 to 56 units per liter (U/L),
aspartate aminotransferase (AST) elevation that is ten or more times higher than the normal AST range of 10 to 40 U/L,
alkaline phosphatase (ALP) elevation that is ten or more times higher than the normal ALP range of 30 to 130 U/L,
lactate dehydrogenase (LDH) elevation that is ten or more times higher than the normal LDH range of 140 to 280 U/L,
creatine kinase (CK) elevation that is ≥3 times greater than upper limits of the normal CK range of 35-175 U/L,
D-dimer elevation that is a level of D-dimer of 500 nanograms per milliliter (mL) or higher, prothrombin time (PT) elevation of higher than the upper range of 11 to 13.5 seconds that indicates that it takes blood longer than usual to clot. Conversely, if the PT number is less than the lower range that indicates that blood clots more quickly than normal.
GM-CSF elevation of three or more times higher than 10 pg per milliliter of GM-CSF,
MCP-1 elevation of two or more times higher than 69.5-175.2 pg/mL of MCP-1,
IP10 elevation of ten or more times higher than 41.5 pg/ml of IP10,
MIP1 alpha (also called CCL3) elevation of >10 pg/mL,
IL-6 elevation of 3 times higher than the upper range of 5-15 pg/ml IL-6,
albumin reduction of below 3.4 grams per deciliter (g/dL),
GM-CSF+CD4+ T cell elevation measured as a percentage of about >3.0% to about 45% of GM-CSF+CD4+ T cells from CD45+CD3+CD4+ T cells isolated from peripheral blood compared to a percentage of about 0% to about 3.0% of GM-CSF+CD4+ T cells from CD45+CD3+CD4+ T cells isolated from peripheral blood of healthy control subjects,
IL-6+CD4+ T cell elevation measured as a percentage of about >1.0% to about 15% of IL-6+CD4+ T cells from CD45+CD3+CD4+ T cells isolated from peripheral blood compared to a percentage of about 0% to about 1.0% of IL-6+CD4+ T cells from CD45+CD3+CD4+ T cells isolated from peripheral blood of healthy control subjects,
INF-γ+GM-CSF+CD4+ T cell elevation measured as a percentage of about >1.0% to about 12.5% of INF-γ+GM-CSF+CD4+ T cells from CD45+CD3+CD4+ T cells isolated from peripheral blood compared to a percentage of about 0% to about 1.0% of INF-γ+GM-CSF+CD4+ T cells from CD45+CD3+CD4+ T cells isolated from peripheral blood of healthy control subjects,
CD14+CD16+ monocyte elevation measured as a percentage of about >10% to about 60% of CD14+CD16+ monocytes from CD45+ monocytes isolated from peripheral blood compared to a percentage of about 0% to 10% of CD14+CD16+ monocytes from CD45+ monocytes isolated from peripheral blood of healthy control subjects,
GM-CSF+CD14+ monocyte elevation measured as a percentage of about >1.25% to about 10% of GM-CSF+CD14+ monocytes from CD14+ monocytes isolated from peripheral blood compared to a percentage of about 0% to about 1.25% of GM-CSF+CD14+ monocytes from CD14+ monocytes isolated from peripheral blood of healthy control subjects,
GM-CSF+CD14+ monocyte elevation measured as a level of about >5×10⁶/L to 35×10⁶/L of GM-CSF+CD14+ monocytes from CD14+ monocytes isolated from peripheral blood compared to a level of about 0×10⁶/L to about 5×10⁶/L of GM-CSF+CD14+ monocytes from CD14+ monocytes isolated from peripheral blood of healthy control subjects,
IL-6+CD14+ monocyte elevation measured as a percentage of about >2.5% to about 20% of IL-6+CD14+ monocytes from CD14+ monocytes isolated from peripheral blood compared to a percentage of about 0% to about 2.5% of IL-6+CD14+ monocytes from CD14+ monocytes isolated from peripheral blood of healthy control subjects, and/or
IL-6+CD14+ monocyte elevation measured as a level of about 10×10⁶/L to 50×10⁶/L of IL-6+CD14+ monocytes from CD14+ monocytes isolated from peripheral blood compared to a level of about 0×10⁶/L to about 9×10⁶/L of IL-6+CD14+ monocytes from CD14+ monocytes isolated from peripheral blood in healthy control subjects.

Additional clinical indicators/markers for a subject being “at high risk for developing CRS” and “at high risk of CRS related inflammatory lung injury” are the person having one or more of the following features: (i) hypotension or shock, i.e., measurement of systolic/diastolic that is less than 90/60 millimeters of mercury (mmHg) or patient requires vasopressors (also called “pressors” herein), (ii) hypoxemia value of arterial oxygen of under 60 mmHg, a pulse oximeter reading (SpO2) of less than or equal to 94% and/or patient requires supplemental oxygen (low-flow oxygen support required for patient in severe condition and high-flow oxygen support, non-invasive positive pressure ventilation (NIPPV)) required for patient in critical state, (iii) a radiological progression of pneumonia shown in chest radiographs as multifocal consolidation, predominantly in the lower lung zone and shown on CT images as ground-glass opacity (GGO), as main findings, and reticulation is noted after the 2nd week. Radiologic findings are usually normal initially or consist of minimal interstitial edema and pleural effusion is common, and/or (iv) multi-organ dysfunction/failure. In some subjects, the radiological findings rapidly progress to bilateral airspace consolidation and fulminant respiratory deterioration within 48 hours and/or (iv) ARDS (acute respiratory distress syndrome) which is demonstrated radiologically by a diffuse lung damage; a rapidly progressive pneumonia results in ARDS.

In specific embodiments of the herein provided therapeutic methods, the GM-CSF antagonist is anti-hGM-CSF antibody Lenzilumab. In some embodiments, the GM-CSF antagonist is chimeric GM-CSF neutralizing antibody KB002 or mouse neutralizing human GM-CSF antibody LMM102. In another embodiment, the GM-CSF antagonist is an anti-GM-SCF antibody selected from the group consisting of Namilumab, Otilimab, Gimsilumab, and TJM2 (TJ003234). In an embodiment, the GM-CSF antagonist is anti-GM-CSF receptor antibody Mavrilimumab. In particular embodiments, a pharmaceutical composition comprising a therapeutically effective amount of a GM-CSF antagonist, e.g., lenzilumab, is administered intravenously to the subject. In some embodiments the methods of treatment comprising administering a pharmaceutical composition comprising a therapeutically effective amount of a GM-CSF antagonist to a subject in need thereof, further comprise administering an anti-viral to the subject. In an embodiment, the anti-viral agent is administered to the subject by any suitable route, as described herein. In specific embodiments, the anti-viral agent is administered intravenously to the subject. In another embodiment, the anti-viral agent is administered orally to the subject. In some embodiments, the anti-viral agent is selected from the group consisting of Aribidol (umifenovir), Favilavir, APN01, defensin mimetic Brilacidin, CCR5 antagonist leronlimab, Remdesivir (GS-5734), GS-441524, Galidesivir (BCX4430), Molnupiravir (MK-4482/EIDD-2801), and MK-7110 (CD24Fc) and combinations thereof. In various embodiments, the herein provided methods further comprise administering a therapeutically effective amount of an anti-viral agent to the subject. In specific embodiments, the therapeutically effective amount of the anti-viral agent is administered intravenously to the subject. In certain embodiments, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In another embodiment, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the inhibitor of HIV-1 protease is lopinavir or a combination of lopinavir and ritonavir (Lopimune; Aluvia). In another embodiment, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In some embodiments, the anti-viral agent is SARS-CoV neutralizing antibody CR3022 that binds and neutralizes a receptor binding domain (RBD) of S-protein of SARS-CoV-2. In an embodiment, the methods provided herein further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In a specific embodiment, the GM-CSF antagonist is anti-hGM-CSF antibody Lenzilumab. In another embodiment, the methods provided herein further comprise administering to the subject a therapeutically effective amount of a (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In some embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

### Pharmaceutical Compositions

Described herein are pharmaceutical compositions comprising compounds of the invention and one or more pharmaceutically acceptable carriers and methods of administering them. “Pharmaceutically acceptable carriers” include any excipient which is nontoxic to the cell or mammal being exposed thereto at the dosages and concentrations employed. The pharmaceutical composition may include one or more therapeutic agents.

Thus, as used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In an embodiment, pharmaceutical compositions containing the therapeutic agent or agents described herein, can be, in one embodiment, administered to a subject by any method known to a person skilled in the art, such as, without limitation, orally, parenterally, transnasally, transmucosally, subcutaneously, transdermally, intramuscularly, intravenously, intraarterially, intra-dermally, intra-peritoneally, intra-ventricularly, intra-cranially, intra-vaginally, or intra-tumorally.

Carriers may be any of those conventionally used, as described above, and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. In one embodiment, water, preferably bacteriostatic water, is the carrier when the pharmaceutical composition is administered intravenously or intratumorally. Saline solutions and aqueous dextrose and glycerol solutions can also be employed as liquid carriers, particularly for injectable solutions.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include, without limitation, physiological saline, bacteriostatic water, Cremophor EL™ (BASF, Parsippany, N.J.) or phosphate buffered saline (PBS). The composition should be sterile and should be fluid to the extent that easy syringeability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as appropriate, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The compositions and formulations as described herein may be administered alone or with other biologically-active agents. Administration can be systemic or local, e.g., through portal vein delivery to the liver. In addition, it may be advantageous to administer the composition into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant.

Moreover, “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable” also includes those carriers approved by a regulatory agency of the Federal or a state government or listed in the U.S. Pharmacopeia or other generally recognized pharmacopeia for use in animals and, more particularly, in humans.

### Effective Doses

Effective doses of the pharmaceutical compositions of the present invention, for treatment of conditions or diseases vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The pharmaceutical compositions of the invention thus may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound of the invention that will be effective in the treatment of a particular disorder or condition, including 2019 coronavirus (SARS-CoV-2) infection, also will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. In one embodiment, the dosage of the GM-CSF antagonist, the anti-viral agent and the oxygen transporter will be within the range of about 0.01-about 1000 mg/kg of body weight. In another embodiment, the dosage will be within the range of about 0.1 mg/kg to about 100 mg/kg. In another embodiment, the dosage will be within the range of about 1 mg/kg to about 10 mg/kg. In an embodiment, the dosage is about 10 mg/kg. In another embodiment, the dosage is 10 mg/kg.

The compound or composition of the invention may be administered only once, or it may be administered multiple times. For multiple dosages, the composition may be, for example, administered three times a day, twice a day, once a day, once every two days, twice a week, weekly, once every two weeks, or monthly.

In an embodiment, the dosage is administered twice daily. In an embodiment, the dosage is administered for four weeks. In an embodiment, the dosage is 10 mg/kg and is administered twice daily for four weeks. The dosage may be administered for 1 week, ten days, two weeks, three weeks, four weeks, six weeks, eight weeks or more, as needed to achieve the desired therapeutic effect. Moreover, effective doses may be extrapolated from dose-response curves derived from in vitro or animal model test bioassays or systems.

In particular embodiments of the herein provided therapeutic methods for treating a subject infected with 2019 coronavirus (SARS-CoV-2), a pharmaceutical composition comprising a therapeutically effective amount of a GM-CSF antagonist is administered to the subject at a dose of from 1200 mg to 1800 mg over 24 hours. In a specific embodiment, the GM-CSF antagonist is neutralizing anti-hGM-CSF antibody Lenzilumab. In an embodiment, the GM-CSF antagonist is administered at a dose of 400 mg every 8 hours over 24 hours. In another embodiment, the GM-CSF antagonist is administered at a dose of 600 mg every 12 hours over 24 hours. In a particular embodiment, the GM-CSF antagonist is administered at a dose of 600 mg every 8 hours over 24 hours for one day. In an embodiment, the administration over 24 hours comprises a total of three doses. In another embodiment, the GM-CSF antagonist is administered at a dose of 800 mg every 12 hours for a total of two doses over 24 hours for one day. In a certain embodiment, the GM-CSF antagonist is administered at a dose of 1800 mg as a single dose for one day. In each of the above-described embodiments, the GM-CSF antagonist is administered intravenously to the subject. In an embodiment, the pharmaceutical composition comprises Lenzilumab in a dose of 400 mg. In a particular embodiment, the pharmaceutical composition comprises Lenzilumab in a dose of 600 mg. In another embodiment, the pharmaceutical composition comprises Lenzilumab in a dose of 800 mg. In still another embodiment, the pharmaceutical composition comprises Lenzilumab in a dose of 1800 mg. In the above-described embodiments, the pharmaceutical composition comprising Lenzilumab is administered intravenously to the subject.

In an embodiment of the herein provided therapeutic methods, the therapeutically effective amount of a GM-CSF antagonist is administered within 48-72 hours of SARS-CoV-2 infection symptom onset. In some embodiments, the therapeutically effective amount of a GM-CSF antagonist is administered when a subject has CRS, is at high risk of developing CRS, or is at high risk of CRS related inflammatory lung injury, wherein being at high risk of developing CRS at high risk of CRS related inflammatory lung injury is as defined hereinabove and as described in Example 1. In an embodiment, a subject is at high risk of developing CRS or is at high risk of CRS related inflammatory lung injury when the subject has one or more of the clinical indicators set forth in Example 1, including but not limited to, a ferritin elevation of >300 mcg/L.

In a specific embodiment of the therapeutic methods for treating a subject infected with 2019 coronavirus (SARS-CoV-2) provided herein, a pharmaceutical composition comprising a therapeutically effective amount of lenzilumab is administered to the subject at a dose of 600 mg every 8 hours for a total of three doses over 24 hours.

In particular embodiments of the therapeutic methods for treating a subject infected with 2019 coronavirus (SARS-CoV-2) provided herein, a pharmaceutical composition comprising a therapeutically effective amount of an anti-viral agent, e.g., Remdesivir, is administered intravenously at 4-15 μg/ml EC₅₀ for 2019 coronavirus (SARS-CoV-2). In an embodiment, Remdesivir is administered intravenously at a dose of 200 mg on day 1 followed by 100 mg on days 2-10 in single daily infusions. In some embodiments, Remdesivir is administered intravenously daily at a dose of 100 mg/kg for 10 days. In another embodiment, Remdesivir is administered intravenously daily at a dose of 150 mg/kg daily doses for 10 days or up to 14 days. In some embodiments, Remdesivir is administered intravenously daily at a dose of 200 mg/kg daily for 10 days. In certain embodiments, lopinavir-ritonavir, a fixed dose of lopinavir (400 mg) with a low dose of ritonavir (100 mg) is administered orally mg twice a day for 14 days. In an embodiment of the herein provided therapeutic methods, the therapeutically effective amount of a GM-CSF antagonist is administered within 48-72 hours of SARS-CoV-2 infection symptom onset. In some embodiments, the therapeutically effective amount of an anti-viral agent, e.g., Remdesivir is administered when a subject has CRS, is at high risk of developing CRS, or is at high risk of CRS related inflammatory lung injury, wherein being at high risk of developing CRS at high risk of CRS related inflammatory lung injury is as defined hereinabove and as described in Example 1. In an embodiment, a subject is at high risk of developing CRS or is at high risk of CRS related inflammatory lung injury when the subject has one or more of the clinical indicators set forth in Example 1, including but not limited to, a ferritin elevation of >300 mcg/L.

In one aspect, the present invention provides a method for treating a subject infected with 2019 coronavirus (SARS-CoV-2), the method comprising administering to the subject a therapeutically effective amount of a GM-CSF antagonist. In an embodiment, the GM-CSF antagonist is the anti-hGM-CSF antibody Lenzilumab. Lenzilumab (Humanigen, Burlingame, Calif.), a hGM-CSF neutralizing antibody in accordance with embodiments described herein and as described in U.S. Pat. Nos. 8,168,183 and 9,017,674, each of which is incorporated herein by reference in its entirety, is a novel, first in class Humaneered® monoclonal antibody that neutralizes human GM-CSF. In a specific embodiment, the therapeutically effective amount of the GM-CSF antagonist, e.g., lenzilumab, is administered intravenously to the subject. In another embodiment, the GM-CSF antagonist is chimeric GM-CSF neutralizing antibody KB002 or mouse neutralizing human GM-CSF antibody LMM102. In one embodiment, the GM-CSF antagonist is an anti-GM-SCF antibody selected from the group consisting of Namilumab, Otilimab, Gimsilumab, and TJM2 (TJ003234). In a further embodiment, the GM-CSF antagonist is anti-GM-CSF alpha receptor antibody Mavrilimumab. In an embodiment, the above-provided methods further comprise administering a therapeutically effective amount of an anti-viral agent. In particular embodiments, the anti-viral agent is administered intravenously to the subject. In some embodiments, the anti-viral agent is selected from the group consisting of Aribidol (umifenovir), Favilavir, APN01, defensin mimetic Brilacidin, CCR5 antagonist leronlimab, Remdesivir (GS-5734), GS-441524, Galidesivir (BCX4430), Molnupiravir (MK-4482/EIDD-2801), and MK-7110 (CD24Fc) and combinations thereof. In various embodiments, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In certain embodiments, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the inhibitor of HIV-1 protease is lopinavir. In another embodiment, the inhibitor of HIV-1 protease comprises a combination of lopinavir and ritonavir (Lopimune; Aluvia). In some embodiments, the combination of the inhibitor of HIV-1 protease and the second drug comprises the inhibitor of HIV-1 protease, darunavir, and the second drug is an inhibitor of human CYP3A proteins, wherein the inhibitor of human CYP3A proteins is cobicistat. In particular embodiments, the anti-viral agent is SARS-CoV neutralizing antibody CR3022 that binds and neutralizes a receptor binding domain (RBD) of S-protein of SARS-CoV-2. In certain embodiments, the herein provided methods further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In specific embodiments of the methods comprising administering the therapeutically effective amount of an anti-SARS-CoV-2 vaccine, the GM-CSF antagonist administered is anti-hGM-CSF antibody Lenzilumab. In some embodiments of the herein provided methods, the methods further comprise administering to the subject a therapeutically effective amount of a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or a therapeutically effective amount of purified immunoglobulins (pIVIg) from a SARS-CoV-2 inoculated transgenic animal that produces human immunoglobulins and the pIVIg contains polyclonal human antibodies to SARS-CoV-2. In various embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (s/IN-2337) or selgantolimod (GS-9688)). In a particular embodiment, the TLR7 agonist, TLR8 agonist and/or the TLR7/8 dual agonist is administered to a male subject.

In another aspect, the present invention provides a method for treating a subject infected with 2019 coronavirus (SARS-CoV-2), the method comprising administering to the subject a therapeutically effective amount of a GM-CSF antagonist and a therapeutically effective amount of an anti-viral agent. In some embodiments, the GM-CSF antagonist is anti-hGM-CSF antibody Lenzilumab. In specific embodiments, the therapeutically effective amount of the GM-CSF antagonist, e.g., lenzilumab, is administered intravenously to the subject. In certain embodiments, the GM-CSF antagonist is chimeric GM-CSF neutralizing antibody KB002 or mouse neutralizing human GM-CSF antibody LMM102. In various embodiments, the GM-CSF antagonist is an anti-GM-SCF antibody selected from the group consisting of Namilumab, Otilimab, Gimsilumab, and TJM2 (TJ003234). In some embodiments, the GM-CSF antagonist is anti-GM-CSF receptor antibody Mavrilimumab. In an embodiment, the anti-viral agent is selected from the group consisting of Aribidol (umifenovir), Favilavir, APN01, defensin mimetic Brilacidin, CCR5 antagonist leronlimab, Remdesivir (GS-5734), GS-441524, Galidesivir (BCX4430), Molnupiravir (MK-4482/EIDD-2801), and MK-7110 (CD24Fc) and combinations thereof. In another embodiment, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In some embodiments, the anti-viral agent comprises a combination of antiretroviral drugs, wherein each of the antiretroviral drugs is an inhibitor of HIV-1 protease. In some embodiments, the inhibitor of HIV-1 protease is lopinavir. In various embodiments, the inhibitor of HIV-1 protease comprises a combination of lopinavir and ritonavir (LOPIMMUNE). In various embodiments, the anti-viral agent is a SARS-CoV neutralizing antibody that binds and neutralizes a receptor binding domain (RBD) of S-protein of SARS-CoV-2, wherein the SARS-CoV neutralizing antibody is CR3022. In particular embodiments, the herein provided methods further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In specific embodiments of the methods comprising the therapeutically effective amount of an anti-SARS-CoV-2 vaccine, the GM-CSF antagonist administered is anti-hGM-CSF antibody Lenzilumab. In various embodiments of the herein provided methods, the methods further comprise administering to the subject a therapeutically effective amount of a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or a therapeutically effective amount of purified immunoglobulins (pIVIg) from a SARS-CoV-2 inoculated transgenic animal that produces human immunoglobulins and the pIVIg contains polyclonal human antibodies to SARS-CoV-2. In some embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In one aspect, the present invention provides a method for preventing and/or treating inflammation-induced lung injury in a subject in need thereof, the method comprising administering to the subject a therapeutically effective amount of a GM-CSF antagonist. In certain embodiments, the GM-CSF antagonist is anti-hGM-CSF antibody Lenzilumab. In some embodiments, the GM-CSF antagonist is chimeric GM-CSF neutralizing antibody KB002 or mouse neutralizing human GM-CSF antibody LMM102. In various embodiments, the GM-CSF antagonist is an anti-GM-SCF antibody selected from the group consisting of Namilumab, Otilimab, Gimsilumab, and TJM2 (TJ003234). In an embodiment, the GM-CSF antagonist is anti-GM-CSF receptor antibody Mavrilimumab. In particular embodiments, the therapeutically effective amount of the GM-CSF antagonist, e.g., lenzilumab, is administered intravenously to the subject. In some embodiments, the herein provided methods further comprise administering a therapeutically effective amount of an anti-viral agent. In specific embodiments, the anti-viral agent is administered intravenously to the subject. In another embodiment, the anti-viral agent is selected from the group consisting of Aribidol (umifenovir), Favilavir, APN01, defensin mimetic Brilacidin, CCR5 antagonist leronlimab, Remdesivir (GS-5734), GS-441524, Galidesivir (BCX4430), Molnupiravir (MK-4482/EIDD-2801), and MK-7110 (CD24Fc) and combinations thereof. In an embodiment, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In a further embodiment, the anti-viral agent comprises a combination of antiretroviral drugs, wherein each of the antiretroviral drugs is an inhibitor of HIV-1 protease. In an embodiment, the anti-viral agent comprises a combination of an inhibitor of HIV-1 protease and a second drug. In an embodiment, the inhibitor of HIV-1 protease is lopinavir. In another embodiment, the inhibitor of HIV-1 protease comprises a combination of lopinavir and ritonavir (Lopimune; Aluvia). In another embodiment of the herein provided methods, a combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In some embodiments, the anti-viral agent is SARS-CoV neutralizing antibody CR3022 that binds and neutralizes a receptor binding domain (RBD) of S-protein of SARS-CoV-2. In a particular embodiment, the subject is infected with SARS-CoV-2. In certain embodiments, the herein provided methods further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In particular embodiments of the methods comprising administering the therapeutically effective amount of an anti-SARS-CoV-2 vaccine, the GM-CSF antagonist administered is anti-hGM-CSF antibody Lenzilumab. In various embodiments of the herein provided methods, the methods further comprise administering to the subject a therapeutically effective amount of a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or a therapeutically effective amount of purified immunoglobulins (pIVIg) from a SARS-CoV-2 inoculated transgenic animal that produces human immunoglobulins and the pIVIg contains polyclonal human antibodies to SARS-CoV-2. In certain embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In a further aspect, the present invention provides a method for preventing and/or treating inflammation-induced lung injury in a subject in need thereof, the method comprising administering to the subject a GM-CSF antagonist and an anti-viral agent. In particular embodiments, the GM-CSF antagonist, e.g., lenzilumab, is administered intravenously to the subject. In some embodiments, the GM-CSF antagonist is anti-hGM-CSF antibody Lenzilumab. In various embodiments, the GM-CSF antagonist is chimeric GM-CSF neutralizing antibody KB002 or mouse neutralizing human GM-CSF antibody LMM102. In certain embodiments, the GM-CSF antagonist is an anti-GM-SCF antibody selected from the group consisting of Namilumab, Otilimab, Gimsilumab, and TJM2 (TJ003234). In an embodiment, the GM-CSF antagonist is anti-GM-CSF receptor antibody Mavrilimumab. In some embodiments, the anti-viral agent is selected from the group consisting of Aribidol (umifenovir), Favilavir, APN01, defensin mimetic Brilacidin, CCR5 antagonist leronlimab, Remdesivir (GS-5734), GS-441524, Galidesivir (BCX4430), Molnupiravir (MK-4482/EIDD-2801), and MK-7110 (CD24Fc) and combinations thereof. In specific embodiments, the anti-viral agent is administered intravenously to the subject. In certain embodiments, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In various embodiments, the anti-viral agent comprises a combination of antiretroviral drugs, wherein each of the antiretroviral drugs is an inhibitor of HIV-1 protease. In some embodiments, the inhibitor of HIV-1 protease is lopinavir. In further embodiments, the inhibitor of HIV-1 protease comprises a combination of lopinavir and ritonavir (Lopimune; Aluvia). In various embodiments, the anti-viral agent is a SARS-CoV neutralizing antibody CR3022 that binds and neutralizes a receptor binding domain (RBD) of S-protein of SARS-CoV-2. In specific embodiments, the subject is infected with SARS-CoV-2. In various embodiments, the herein provided methods further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In specific embodiments of the methods comprising administering the therapeutically effective amount of an anti-SARS-CoV-2 vaccine, the GM-CSF antagonist administered is anti-hGM-CSF antibody Lenzilumab. In various embodiments of the herein provided methods, the methods further comprise administering to the subject a therapeutically effective amount of a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or a therapeutically effective amount of purified immunoglobulins (pIVIg) from a SARS-CoV-2 inoculated transgenic animal that produces human immunoglobulins and the pIVIg contains polyclonal human antibodies to SARS-CoV-2. In an embodiment, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In one aspect, the present invention provides a method for preventing and/or treating cytokine release syndrome (CRS) and/or toxicity induced by CRS in a subject in need thereof, the method comprising administering to the subject a GM-CSF antagonist. In a particular some embodiment, the GM-CSF antagonist is anti-hGM-CSF antibody Lenzilumab. In particular embodiments, the GM-CSF antagonist, e.g., lenzilumab, is administered intravenously to the subject. In an embodiment, the GM-CSF antagonist is chimeric GM-CSF neutralizing antibody KB002 or mouse neutralizing human GM-CSF antibody LMM102. In some embodiments, the GM-CSF antagonist is an anti-GM-SCF antibody selected from the group consisting of Namilumab, Otilimab, Gimsilumab, and TJM2 (TJ003234). In certain embodiments, the GM-CSF antagonist is anti-GM-CSF receptor antibody Mavrilimumab. In an embodiment, the herein provided methods further comprise administering a therapeutically effective amount of an anti-viral agent. In particular embodiments, the anti-viral agent is administered intravenously to the subject. In some embodiments, the anti-viral agent is selected from the group consisting of Aribidol (umifenovir), Favilavir, APN01, defensin mimetic Brilacidin, CCR5 antagonist leronlimab, Remdesivir (GS-5734), GS-441524, Galidesivir (BCX4430), Molnupiravir (MK-4482/EIDD-2801), and MK-7110 (CD24Fc) and combinations thereof. In another embodiment, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In certain embodiments, the anti-viral agent comprises a combination of antiretroviral drugs, wherein each of the antiretroviral drugs is an inhibitor of HIV-1 protease. In some embodiment, the inhibitor of HIV-1 protease is lopinavir. In an embodiment, the inhibitor of HIV-1 protease comprises a combination of lopinavir and ritonavir (Lopimune; Aluvia). In various embodiments, the anti-viral agent is SARS-CoV neutralizing antibody CR3022 that binds and neutralizes a receptor binding domain (RBD) of S-protein of SARS-CoV-2. In a specific embodiment, the subject is infected with SARS-CoV-2. In an embodiment, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the herein provided methods comprising administering a combination the inhibitor of HIV-1 protease and the second drug, the methods comprise administering the inhibitor of HIV-1 protease, darunavir, and the second drug is an inhibitor of human CYP3A proteins, wherein the inhibitor of human CYP3A proteins is cobicistat. In a specific embodiment, the anti-viral agent is SARS-CoV neutralizing antibody CR3022 that binds and neutralizes a receptor binding domain (RBD) of S-protein of SARS-CoV-2. In a particular embodiment of the herein provided methods, the methods of further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In specific embodiments of the methods comprising administering the therapeutically effective amount of an anti-SARS-CoV-2 vaccine, the GM-CSF antagonist administered is anti-hGM-CSF antibody Lenzilumab. In certain embodiments of the herein provided methods, the methods further comprise administering to the subject a therapeutically effective amount of a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or a therapeutically effective amount of purified immunoglobulins (pIVIg) from a SARS-CoV-2 inoculated transgenic animal that produces human immunoglobulins and the pIVIg contains polyclonal human antibodies to SARS-CoV-2. In particular embodiments, the subject is infected with SARS-CoV-2 or purified immunoglobulins (pIVIg) from a SARS-CoV-2 inoculated transgenic animal that produces human immunoglobulins and the pIVIg contains polyclonal human antibodies to SARS-CoV-2. In particular embodiments, the subject is infected with SARS-CoV-2. In certain embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In another aspect, the present invention provides a method for preventing and/or treating cytokine release syndrome (CRS) and/or toxicity induced by CRS in a subject in need thereof, the method comprising administering to the subject a GM-CSF antagonist and an anti-viral agent. In specific embodiments, the subject in need of prevention and/or treatment of CRS and/or toxicity induced by CRS is a subject infected with 2019 coronavirus (SARS-CoV-2). In a particular embodiment, the GM-CSF antagonist is anti-hGM-CSF antibody Lenzilumab. In particular embodiments, the GM-CSF antagonist, e.g., lenzilumab, is administered intravenously to the subject. In another embodiment, the GM-CSF antagonist is chimeric GM-CSF neutralizing antibody KB002 or mouse neutralizing human GM-CSF antibody LMM102. In some embodiments, the GM-CSF antagonist is an anti-GM-SCF antibody selected from the group consisting of Namilumab, Otilimab, Gimsilumab, and TJM2 (TJ003234). In certain embodiments, the GM-CSF antagonist is anti-GM-CSF receptor antibody Mavrilimumab. In specific embodiments, the anti-viral agent is administered intravenously to the subject. In various embodiments, the anti-viral agent is selected from the group consisting of Aribidol (umifenovir), Favilavir, APN01, defensin mimetic Brilacidin, CCR5 antagonist leronlimab, Remdesivir (GS-5734), GS-441524, Galidesivir (BCX4430), Molnupiravir (MK-4482/EIDD-2801), and MK-7110 (CD24Fc) and combinations thereof. In an embodiment, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In one embodiment, the anti-viral agent comprises a combination of antiretroviral drugs, wherein each of the antiretroviral drugs is an inhibitor of HIV-1 protease. In an embodiment, the inhibitor of HIV-1 protease is lopinavir. In another embodiment, the inhibitor of HIV-1 protease comprises a combination of lopinavir and ritonavir (Lopimune; Aluvia). In some embodiments, the anti-viral agent is SARS-CoV neutralizing antibody CR3022 that binds and neutralizes a receptor binding domain (RBD) of S-protein of SARS-CoV-2. In a specific embodiment, the subject is infected with SARS-CoV-2. In an embodiment, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the herein provided methods comprising administering a combination the inhibitor of HIV-1 protease and the second drug, the methods comprise administering the inhibitor of HIV-1 protease, darunavir, and the second drug is an inhibitor of human CYP3A proteins, wherein the inhibitor of human CYP3A proteins is cobicistat. In a specific embodiment, the anti-viral agent is SARS-CoV neutralizing antibody CR3022 that binds and neutralizes a receptor binding domain (RBD) of S-protein of SARS-CoV-2. In a particular embodiment of the herein provided methods, the methods of further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In specific embodiments of the methods comprising administering the therapeutically effective amount of an anti-SARS-CoV-2 vaccine, the GM-CSF antagonist administered is anti-hGM-CSF antibody Lenzilumab. In certain embodiments of the herein provided methods, the methods further comprise administering to the subject a therapeutically effective amount of a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or a therapeutically effective amount of purified immunoglobulins (pIVIg) from a SARS-CoV-2 inoculated transgenic animal that produces human immunoglobulins and the pIVIg contains polyclonal human antibodies to SARS-CoV-2. In some embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In another aspect, the present invention provides a method for treating a subject infected with a coronavirus (SARS-CoV-2) comprising administering to the subject a therapeutically effective amount of GM-CSF antagonist and a therapeutically effective amount of an oxygen transporter. In specific embodiments, the oxygen transporter is BXT25. In an embodiment, the GM-CSF antagonist is anti-hGM-CSF antibody Lenzilumab. In particular embodiments, the GM-CSF antagonist, e.g., lenzilumab, is administered intravenously to the subject. In another embodiment, the GM-CSF antagonist is chimeric GM-CSF neutralizing antibody KB002 or mouse neutralizing human GM-CSF antibody LMM102. In one embodiment, the GM-CSF antagonist is an anti-GM-SCF antibody selected from the group consisting of Namilumab, Otilimab, Gimsilumab, and TJM2 (TJ003234). In a further embodiment, the GM-CSF antagonist is anti-GM-CSF receptor antibody Mavrilimumab. In an embodiment, the above-provided methods further comprise administering a therapeutically effective amount of an anti-viral agent. In particular embodiments, the anti-viral agent is administered intravenously to the subject. In some embodiments, the anti-viral agent is selected from the group consisting of Aribidol (umifenovir), Favilavir, APN01, defensin mimetic Brilacidin, CCR5 antagonist leronlimab, Remdesivir (GS-5734), GS-441524, Galidesivir (BCX4430), Molnupiravir (MK-4482/EIDD-2801), and MK-7110 (CD24Fc) and combinations thereof. In various embodiments, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In certain embodiments, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the inhibitor of HIV-1 protease is lopinavir. In another embodiment, the inhibitor of HIV-1 protease comprises a combination of lopinavir and ritonavir (Lopimune; Aluvia). In some embodiments, the combination of the inhibitor of HIV-1 protease and the second drug comprises the inhibitor of HIV-1 protease, darunavir, and the second drug is an inhibitor of human CYP3A proteins, wherein the inhibitor of human CYP3A proteins is cobicistat. In particular embodiments, the anti-viral agent is SARS-CoV neutralizing antibody CR3022 that binds and neutralizes a receptor binding domain (RBD) of S-protein of SARS-CoV-2. In certain embodiments, the herein provided methods further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In specific embodiments of the methods comprising administering the therapeutically effective amount of an anti-SARS-CoV-2 vaccine, the GM-CSF antagonist administered is anti-hGM-CSF antibody Lenzilumab. In some embodiments of the herein provided methods, the methods further comprise administering to the subject a therapeutically effective amount of a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or a therapeutically effective amount of purified immunoglobulins (pIVIg) from a SARS-CoV-2 inoculated transgenic animal that produces human immunoglobulins and the pIVIg contains polyclonal human antibodies to SARS-CoV-2. In certain embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In another aspect, the present invention provides a method for treating and/or preventing inflammation-induced lung injury in a subject infected with a coronavirus (SARS-CoV-2) comprising administering to the subject a therapeutically effective amount of GM-CSF antagonist and a therapeutically effective amount of an oxygen transporter. In particular embodiments, the oxygen transporter is BXT25. In an embodiment, the GM-CSF antagonist is anti-hGM-CSF antibody Lenzilumab. In particular embodiments, the GM-CSF antagonist, e.g., lenzilumab, is administered intravenously to the subject. In another embodiment, the GM-CSF antagonist is chimeric GM-CSF neutralizing antibody KB002 or mouse neutralizing human GM-CSF antibody LMM102. In one embodiment, the GM-CSF antagonist is an anti-GM-SCF antibody selected from the group consisting of Namilumab, Otilimab, Gimsilumab, and TJM2 (TJ003234). In a further embodiment, the GM-CSF antagonist is anti-GM-CSF receptor antibody Mavrilimumab. In an embodiment, the above-provided methods further comprise administering a therapeutically effective amount of an anti-viral agent. In some embodiments, the anti-viral agent is selected from the group consisting of Aribidol (umifenovir), Favilavir, APN01, defensin mimetic Brilacidin, CCR5 antagonist leronlimab, Remdesivir (GS-5734), GS-441524, Galidesivir (BCX4430), Molnupiravir (MK-4482/EIDD-2801), and MK-7110 (CD24Fc) and combinations thereof. In various embodiments, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In certain embodiments, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the inhibitor of HIV-1 protease is lopinavir. In another embodiment, the inhibitor of HIV-1 protease comprises a combination of lopinavir and ritonavir (Lopimune; Aluvia). In some embodiments, the combination of the inhibitor of HIV-1 protease and the second drug comprises the inhibitor of HIV-1 protease, darunavir, and the second drug is an inhibitor of human CYP3A proteins, wherein the inhibitor of human CYP3A proteins is cobicistat. In particular embodiments, the anti-viral agent is SARS-CoV neutralizing antibody CR3022 that binds and neutralizes a receptor binding domain (RBD) of S-protein of SARS-CoV-2. In certain embodiments, the herein provided methods further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech), adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In specific embodiments of the methods comprising administering the therapeutically effective amount of an anti-SARS-CoV-2 vaccine, the GM-CSF antagonist administered is anti-hGM-CSF antibody Lenzilumab. In some embodiments of the herein provided methods, the methods further comprise administering to the subject a therapeutically effective amount of a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or a therapeutically effective amount of purified immunoglobulins (pIVIg) from a SARS-CoV-2 inoculated transgenic animal that produces human immunoglobulins and the pIVIg contains polyclonal human antibodies to SARS-CoV-2. In various embodiments, the herein provided methods further comprise administering to the subject a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist and/or the TLR7/8 dual agonist is administered to a male subject.

In one aspect, the present invention provides a method for reducing time to clinical improvement or time to recovery of a subject infected with 2019 coronavirus (SARS-CoV-2), the method comprising administering to the subject a pharmaceutical composition comprising a therapeutically effective amount of a GM-CSF antagonist, wherein the time to clinical improvement or time to recovery of the subject is reduced by at least 40% compared to the time to clinical improvement or time to recovery of a control subject treated with standard of care and is not administered a GM-CSF antagonist, wherein the subject and the control subject each have severe COVID-19 pneumonia. In an embodiment of the provided method, wherein the clinical improvement comprises at least two points on an 8-point ordinal clinical outcome scale and time to recovery comprises obtaining/reaching a 6, 7, or 8 score wherein the 8-point ordinal outcome scale is a clinical status of the subject consisting of scores: 1) death; 2) hospitalized, on invasive mechanical ventilation or extracorporeal membrane oxygenation (ECMO); 3) hospitalized, on non-invasive ventilation or high flow oxygen devices; 4) hospitalized, requiring supplemental oxygen; 5) hospitalized, not requiring supplemental oxygen and requiring ongoing medical care; 6) hospitalized, not requiring supplemental oxygen and no longer requiring ongoing medical care; 7) not hospitalized and having a limitation of activities; and 8) not hospitalized and having no limitations of activities. In another embodiment, the medical care of the standard of care is COVID-19 related medical care and/or medical care not related to COVID-19. In an embodiment, the standard of care of the control subject comprises administration of a therapeutically effective amount of an anti-viral agent, a steroid, hydroxychloroquine (HCQ), an anti-interleukin-6 (IL-6) receptor monoclonal antibody, azithromycin, an immunoglobulin, intravenous immunoglobulin (IVIG), a convalescent plasma comprising COVID 19 immune serum, a statin, and combinations thereof. In an embodiment of the provided methods, the anti-viral agent comprises Remdesivir (GS-5734), GS-441524, ribavirin, Aribidol (umifenovir), Favilavir, APN01, defensin mimetic Brilacidin, CCR5 antagonist leronlimab (PRO140), Galidesivir (BCX4430), GS-441524, Molnupiravir (MK-4482/EIDD-2801), and MK-7110 (CD24Fc) and combinations thereof. In certain embodiments, the anti-IL6 receptor monoclonal antibody comprises tocilizumab or sarilumab. In a particular embodiment, the IVIG comprises human immune globulin g (OCTAGAM® 10% Octapharma USA, Hoboken, N.J.)). In an embodiment, the human immune globulin g (OCTAGAM® is administered intravenously at a dose of 0.5 g/kg daily for 3 days.

In an embodiment of the herein provided methods, the ratio of oxygen saturation by pulse oximetry (SpO₂) to fraction of inspired oxygen (FiO2) of the subject administered the GM-CSF antagonist improves within one day of administration of the GM-CSF antagonist compared to the (SpO₂)/(FiO2) of the control subject. ARDS is defined by the Berlin Criteria as an SpO2/FiO2<315 or as a PaO2/FiO2 ratio <300. In a particular embodiment, the subject administered the GM-CSF antagonist has ARDS. In certain embodiments of the methods described herein, the acute respiratory distress syndrome (ARDS) of the subject administered the GM-CSF antagonist improves within one day of administration of the GM-CSF antagonist and ARDS is reduced over time by at least day 4 post-GM-CSF antagonist administration compared to the ARDS improvement and reduction over time by at least day 4 of the control subject, wherein reduction in the ARDS comprises a change in a ratio of SpO2/FiO2 from less than 315 to a ratio of SpO2/FiO2 315 or higher. In an embodiment, the subject administered the GM-CSF antagonist has an elevated serum C-reactive protein (CRP) level. In some embodiments, the elevated serum C-reactive protein (CRP) level of the subject administered the GM-CSF antagonist is reduced by at least 50% within one to two days of administration of the GM-CSF antagonist compared to reduction in the elevated serum CRP level, over the same timeframe, in the control subject, wherein the elevated serum CRP level is above the upper limit of normal (>8.0 mg/L).

In an embodiment, the subject administered the GM-CSF antagonist has an absolute lymphocyte counts (ALC) of 0.95-3.07×10⁹/L or less before administration of the GM-CSF antagonist, and after the administration, the subject has a change (an increase) in the absolute lymphocyte counts (ALC). Examples 8 and 9 provide the ALCs of subjects before administration of an GM-CSF antagonist; one subject had an ALC as low as 0.62×10⁹/L and another had an ALC of 0.89×10⁹/L prior to administration of the GM-CSF antagonist In a particular embodiment, the change in the absolute lymphocyte counts (ALC) of the subject administered the GM-CSF antagonist is an ALC of at least 1000-fold greater compared to the ALC of the control subject.

In another embodiment, the time to discharge of the subject is 40%-50% faster in the subject administered the GM-CSF antagonist compared to the time to discharge of the control subject. In an embodiment, serum IL-6 concentration of the subject administered the GM-CSF antagonist is elevated, i.e., outside the normal upper limit of serum IL-6 concentration. In certain embodiments, the serum IL-6 concentration of the subject administered the GM-CSF antagonist is reduced by at least 50% in the subject on or by day 4 after administration of the GM-CSF antagonist compared to the reduction in the serum IL-6 concentration of the subject on or by day 4 of the control subject. In an embodiment of the herein described methods, incidence of invasive mechanical ventilation (IMV) and/or death of the subject administered the GM-CSF antagonist is reduced by 80% on a relative basis and is reduced by 33% on an absolute risk reduction compared to the IMV and/or death of the control subject, wherein invasive mechanical ventilation-free survival of a subject administered the GM-CSF antagonist is increased by 40% to 80% on an relative basis compared to the invasive mechanical ventilation-free survival of the control subject. In certain embodiments, relative risk of invasive mechanical ventilation (IMV) and/or death of the subject administered the GM-CSF antagonist is reduced by 30% or more compared to the IMV and/or death of the invasive mechanical ventilation-free survival of control subject treated with standard of care and not administered a GM-CSF antagonist. In some embodiments of the methods provided herein, the COVID-19 pneumonia is severe COVID-19 pneumonia as determined by radiographic assessment or by low-flow oxygen requirement. In an embodiment, the COVID-19 pneumonia is critical COVID-19 pneumonia as determined by the need for high-flow oxygen or non-invasive positive pressure ventilation support. In certain embodiments, the time to clinical improvement or time to recovery of the subject administered the GM-CSF antagonist is reduced by at least 50% compared to the time to clinical improvement or time to recovery of a control subject.

In an embodiment, the subject administered the GM-CSF antagonist and the control subject each have clinical and/or biomarker evidence for increased risk of progression to respiratory failure. In particular embodiments of the methods provided herein, the clinical evidence for increased risk of progression to respiratory failure comprises fever, CRP >100 mg/L, lymphocytopenia, hypotension, shock, capillary leak syndrome, pulmonary edema, disseminated intravascular coagulation, a hypoxemia value of arterial oxygen of under 60 mmHg, a pulse oximeter reading (SpO2) of less than or equal to 94%, the subject requiring supplemental oxygen, a radiological progression of pneumonia shown in chest radiographs as multifocal consolidation and/or shown on CT images as ground-glass opacity, multi-organ dysfunction/failure, and/or ARDS shown radiologically by a diffuse lung damage. As described above, a subject is defined as having ARDS when the subject's SpO2/FiO2 ratio <315 (or PaO2/FiO2 ratio <300). In certain embodiments, the biomarker evidence for increased risk of progression to respiratory failure comprises abnormal levels of liver enzymes, coagulation markers, albumin, creatinine phosphokinase and lactate dehydrogenase; elevated levels above the upper limit of normal levels of at least one cytokine/chemokine selected from the group consisting of GM-CSF, G-CSF, MCD, IL-1α, IFN-γ, IL-7, FMS-related tyrosine kinase 3 ligand (FLT-3L), IL-1rα, IL-6, and IL-12p70, MCP-1, IP10, MIP1α, and MIP1β; and/or a ferritin level of >300 mcg/L.

In embodiments of the methods provided herein, the subject administered the GM-CSF antagonist and the control subject each have at least one risk factor associated with poor outcome selected from the group consisting of age at or over 60 years, smoking history, cardiovascular disease, diabetes, chronic kidney disease, chronic lung disease, high BMI, and at least one elevated biomarker inflammatory marker. In particular embodiments, the at least one elevated biomarker inflammatory marker comprises CRP, serum ferritin, D-dimer, IL-6 or lactate dehydrogenase. In particular embodiments of the methods provided herein, the subject and the control subject each require oxygen supplementation without mechanical ventilation.

In an embodiment of the methods provided herein, the pharmaceutical composition comprising a therapeutically effective amount of a GM-CSF antagonist is administered at a total dose of from 1200 mg to 1800 mg over 24 hours. In a particular embodiment, the GM-CSF antagonist is neutralizing anti-hGM-CSF antibody Lenzilumab. In an embodiment, the GM-CSF antagonist is administered at a dose of 400 mg every 8 hours for a total of three doses over 24 hours. In some embodiments, the GM-CSF antagonist is administered at a dose of 600 mg every 8 hours for a total of three doses over 24 hours for one day. In certain embodiments, the GM-CSF antagonist is administered at a dose of 800 mg every 12 hours for a total of two doses over 24 hours for one day. In an embodiment, the GM-CSF antagonist is administered as a single dose of 1800 mg. In some embodiments, the GM-CSF antagonist is chimeric GM-CSF neutralizing antibody KB002 or mouse neutralizing human GM-CSF antibody LMM102. In an embodiment, the GM-CSF antagonist is an anti-GM-SCF antibody selected from the group consisting of Namilumab, Otilimab, Gimsilumab, and TJM2 (TJ003234). In another embodiment, the GM-CSF antagonist is anti-GM-CSF receptor antibody Mavrilimumab.

In certain embodiments of the methods provided herein, the methods further comprise administering a therapeutically effective amount of an anti-viral agent to the subject administered the GM-CSF antagonist and/or to the control subject. In particular embodiments, the anti-viral agent comprises Remdesivir (GS-5734), GS-441524, ribavirin, Aribidol (umifenovir), Favilavir, APN01, defensin mimetic Brilacidin, CCR5 antagonist leronlimab (PRO140), Galidesivir (BCX4430), GS-441524, Molnupiravir (MK-4482/EIDD-2801), and MK-7110 (CD24Fc) and combinations thereof. In some embodiments, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In particular embodiments, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the inhibitor of HIV-1 protease is lopinavir or a combination of lopinavir and ritonavir (Lopimune; Aluvia). In some embodiments, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat.

In a particular embodiment of the methods provided herein, the GM-CSF antagonist is lenzilumab and the antiviral agent administered to the subject administered the lenzilumab and/or to the control subject is Remdesivir (GS-5734), the time to recovery of the subject administered the lenzilumab and the anti-viral agent is reduced by at least 40% compared to the time to recovery of the control subject, administered the antiviral agent without administration of lenzilumab. In an embodiment, the time to recovery of the subject administered the lenzilumab and the anti-viral agent is reduced by at least 50% compared to the time to recovery of the control subject. In some embodiments, wherein the GM-CSF antagonist is lenzilumab and the antiviral agent administered to the subject administered the lenzilumab and/or to the control subject is a combination of lopinavir and ritonavir (Lopimune; Aluvia), the time to recovery of the subject administered the lenzilumab and the anti-viral agent is reduced by at least 40% compared to the time to recovery of the control subject administered the antiviral agent without administration of lenzilumab. In an embodiment, the time to recovery of the subject administered the lenzilumab and the anti-viral agent is reduced by at least 50% compared to the time to recovery of the control subject administered the antiviral agent without administration of lenzilumab. In some embodiments, one or more of the antiviral agents described herein is administered in addition to Remdesivir (GS-5734).

In an embodiment of the methods provided herein, the methods further comprise administering a therapeutically effective amount of an anti-viral agent, a steroid, hydroxychloroquine (HCQ), azithromycin, an anti-interleukin-6 (IL-6) receptor monoclonal antibody, an immunoglobulin, intravenous immunoglobulin (IVIG), a statin, and combinations thereof to the subject administered the GM-CSF antagonist. In some embodiments, the anti-viral agent comprises Remdesivir (GS-5734), GS-441524, ribavirin, Aribidol (umifenovir), Favilavir, APN01, defensin mimetic Brilacidin, CCR5 antagonist leronlimab (PRO140), Galidesivir (BCX4430), Molnupiravir (MK-4482/EIDD-2801), and MK-7110 (CD24Fc) and combinations thereof and combinations thereof. In another embodiment, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In an embodiment, the IVIG comprises human immune globulin g. In some embodiments, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the inhibitor of HIV-1 protease is lopinavir or a combination of lopinavir and ritonavir (Lopimune; Aluvia). In some embodiments of the herein provided methods, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In certain embodiments, the herein provided methods further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech) adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In a particular embodiment, the GM-CSF antagonist is neutralizing anti-hGM-CSF antibody Lenzilumab. In certain embodiments of the herein provided methods, the methods further comprise administering to the subject a therapeutically effective amount of a (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In another embodiment, the provided methods further comprise administering a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod VTX-2337) or selgantolimod (GS-9688)). In a particular embodiment, the TLR7 agonist, TLR8 agonist or a TLR7/8 dual agonist is administered to a male subject.

In another aspect, the present invention provides a method for treating a subject infected with 2019 coronavirus (SARS-CoV-2) for a time period beyond an initial acute hyper-inflammatory period, the method comprising administering to the subject a pharmaceutical composition comprising a therapeutically effective amount of a GM-CSF antagonist. In an embodiment of this method, the time period beyond the initial acute hyper-inflammatory period is from 21 days to 13 weeks after onset of the initial acute hyper-inflammatory period. In some embodiments, the initial acute hyper-inflammatory period occurs about 5 to 12 days after onset of symptoms of infection with SARS-CoV-2. In certain embodiments, the symptoms of infection with SARS-CoV-2 occur 2 to 14 day after exposure to SARS-CoV-2, wherein the symptoms of infection with SARS-CoV-2 comprise fever, chills, cough without fever, shortness of breath, difficulty breathing, fatigue, muscle aches, body aches, headache, back ache, loss of taste and/or smell, sore throat, congestion, runny nose, nausea, vomiting, diarrhea, abdominal pain, or combinations thereof. In a particular embodiment, the onset of the initial acute hyper-inflammatory period is determined by plasma of the subject comprising below normal lower level of absolute lymphocyte counts, elevated level of CRP, serum ferritin, D-dimer, IL-6, liver enzymes, albumin, creatinine phosphokinase, lactate dehydrogenase, inflammatory cytokine, troponin, myeloid cells, or combinations thereof. In some embodiments, the elevated levels of the inflammatory cytokine comprise elevated levels of IL-6, G-CSF, GM-CSF, MCP-1, MIP-1α, MIP-1β, MIG, IP-10, MDC, IL-1α, IL-8, IL-10, IFN-γ, IL-7, FLT-3L, IL-1rα, IL-12p70 or combinations thereof. In an embodiment, the below normal lower level of absolute lymphocyte counts (ALC) comprises an ALC of 0.95×10⁹/L or less, wherein the below normal lower level of ALC occurs about 4 to 8 days after onset of symptoms of infection with SARS-CoV-2. In certain embodiments, the elevated levels of the myeloid cells comprise CD14+ myeloid cells. In some embodiments, the onset of the initial acute hyper-inflammatory period is further determined by the subject having dyspnea and hypoxia, wherein the dyspnea occurs about 5 to 9 days after onset of symptoms of infection with SARS-CoV-2. In various embodiments, the onset of the initial acute hyper-inflammatory period is further determined by the subject manifesting with Acute Respiratory Distress Syndrome (ARDS), wherein the ARDS occurs about 8 to 12 days after onset of symptoms of infection with SARS-CoV-2. In an embodiment, the ARDS further comprises the subject having severe lung inflammation and lung damage. In some embodiments, the onset of the initial acute hyper-inflammatory period is further determined by abnormal lung computed tomography (CT) scans. In a particular embodiment of the herein described methods, the GM-CSF antagonist is anti-hGM-CSF antibody lenzilumab. In an embodiment, the pharmaceutical composition comprising lenzilumab is administered at a dose of from 1200 mg to 1800 mg over 24 hours. In certain embodiments, the pharmaceutical composition comprising lenzilumab is administered at a dose of 1800 mg over 24 hours. In some embodiments, the GM-CSF antagonist is an anti-GM-CSF antibody selected from the group consisting of Namilumab, Otilimab, Gimsilumab, and TJM2 (TJ003234). In some embodiments, the pharmaceutical composition comprising Namilumab, Otilimab, Gimsilumab, or TJM2 (TJ003234) is administered at a dose of from 1200 mg to 1800 mg over 24 hours. In an embodiment, the pharmaceutical composition comprising Namilumab, Otilimab, Gimsilumab, or TJM2 (TJ003234) is administered at a dose of 1800 mg over 24 hours. In another embodiment, the GM-CSF antagonist is anti-GM-CSF alpha receptor antibody Mavrilimumab. In an embodiment, the pharmaceutical composition comprising Mavrilimumab is administered at a dose of from 1200 mg to 1800 mg over 24 hours. In certain embodiments, the pharmaceutical composition comprising Mavrilimumab is administered at a dose of 1800 mg over 24 hours. In certain embodiments, the subject has ARDS, COVID-19 pneumonia, severe hypoxemia, lymphopenia on complete blood count, bilateral infiltrates on chest x-ray, diffuse ground glass opacities on lung CT scan, a bacterial respiratory tract infection, a fungal respiratory tract infection, mild transaminitis on liver function tests or combinations thereof prior to administration of the pharmaceutical composition. In various embodiments, the subject is administered high-flow supplemental oxygen. In an embodiment, the subject is treated with a standard of care prior to administration of the pharmaceutical composition, where the standard of care comprises administration of an antibacterial agent, an antifungal agent, hydroxychloroquine and zinc, a corticosteroid or combinations thereof. In an embodiment, the high-flow supplemental oxygen administration is reduced to low-flow nasal cannula after administration of the pharmaceutical composition. In a particular embodiment, time to clinical improvement or time to recovery of the subject is accelerated to one week after administration of the pharmaceutical composition, the recovery comprising improvement in lymphopenia, decreased supplemental oxygen administration from high-flow to low-flow; improved mobility and accelerated time to discharge, compared to a lack of time to clinical improvement or time to recovery of the same subject treated with standard of care for 12 weeks, wherein the same subject was not administered a GM-CSF antagonist during treatment with the standard of care. In an embodiment, the accelerated time to discharge is 16 days after administration of the pharmaceutical composition. In some embodiments, the subject has a comorbidity, wherein the comorbidity comprises age over 65 years, male sex, type II diabetes, hypertension, cardiovascular disease, heart disease, coronary artery disease, obesity, obstructive lung disease, chronic obstructive pulmonary disease, reactive airway disease, chronic kidney disease, kidney transplantation or combinations thereof. In certain embodiments, the subject having the comorbidity is refractory to corticosteroids. In another embodiment, the subject infected with 2019 coronavirus (SARS-CoV-2) for a time period beyond an initial acute hyper-inflammatory period is refractory to corticosteroids. In an embodiment of the methods provided herein, the methods further comprise administering a therapeutically effective amount of an anti-viral agent, a steroid, hydroxychloroquine (HCQ), azithromycin, an anti-interleukin-6 (IL-6) receptor monoclonal antibody, an immunoglobulin, intravenous immunoglobulin (IVIG), a statin, and combinations thereof to the subject administered the GM-CSF antagonist. In some embodiments, the anti-viral agent comprises Remdesivir (GS-5734), GS-441524, ribavirin, Aribidol (umifenovir), Favilavir, APN01, defensin mimetic Brilacidin, CCR5 antagonist leronlimab (PRO140), Galidesivir (BCX4430), Molnupiravir (MK-4482/EIDD-2801), and MK-7110 (CD24Fc) and combinations thereof and combinations thereof. In another embodiment, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In an embodiment, the IVIG comprises human immune globulin g. In some embodiments, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the inhibitor of HIV-1 protease is lopinavir or a combination of lopinavir and ritonavir (Lopimune; Aluvia). In some embodiments of the herein provided methods, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat. In certain embodiments, the herein provided methods further comprise administering to the subject a therapeutically effective amount of an anti-SARS-CoV-2 vaccine selected from the group consisting of an intranasal SARS-CoV-2 vaccine (Altimmune), INO-4800 (Inovio Pharma and Beijing Advaccine Biotechnology Company), APN01 (APEIRON Biologics), mRNA-1273 vaccine (Moderna and the Vaccine Research Center), nucleoside modified mNRA BNT162b2 Tozinameran (INN) (Pfizer-BioNTech) adenovirus-based vaccine AZD1222 (recombinant ChAdOx1 adenoviral vector encoding the SARS-CoV-2 spike protein antigen; Oxford-AstraZeneca), Covishield (ChAdOx1_nCoV19) recombinant ChAdOx1 adenoviral vector encoding SARS-CoV-2 spike protein antigen (Serum Institute of India), SARS-CoV-2 Vaccine (Vero Cell), Inactivated (lnCoV) (Sinopharm/BIBP), SARS-CoV-2 Vaccine (Vero Cell), Inactivated (Sinovac), Ad26.COV2.S recombinant, replication-incompetent adenovirus type 26 (Ad26) vectored vaccine encoding SARS-CoV-2) Spike (S) protein (Janssen Pharmaceuticals Companies of Johnson & Johnson), Sputnik V Human Adenovirus Vector-based Covid-19 vaccine (The Gamaleya National Center), Ad5-nCoV Recombinant Novel Coronavirus Vaccine (Adenovirus Type 5 Vector) (CanSinoBIO), EpiVacCorona Peptide antigen vaccine (Vector State Research Centre of Viralogy and Biotechnology, Russia), Recombinant Novel Coronavirus Vaccine (CHO) (Zhifei Longcom, China), SARS-CoV-2 Vaccine, Inactivated (Vero Cell) (IMBCAMS, China), Inactivated SARS-CoV-2 Vaccine (Vero Cell) (Sinopharm/WIBP), an avian coronavirus infectious bronchitis virus (IBV) vaccine (MIGDAL Research Institute), a modified horsepox virus vaccine TNX-1800 (Tonix Pharmaceuticals), a recombinant subunit vaccine based on trimeric S protein (S-Trimer) of the SARS-CoV-2 coronavirus (Clover Pharmaceuticals), an oral recombinant coronavirus vaccine (Vaxart), a linear DNA vaccine based on (i) the entire spike gene of the coronavirus or (ii) based on the antigenic portions of the coronavirus protein (Applied DNA Sciences and Takis Biotech), SARS-Cov-2 coronavirus vaccine NVX-CoV2373 (Novavax), an intramuscular vaccine INO-4700 (GLS-5300) (Inovio Pharma and GeneOne Life Science), and combinations thereof. In a particular embodiment, the GM-CSF antagonist is neutralizing anti-hGM-CSF antibody Lenzilumab. In certain embodiments of the herein provided methods, the methods further comprise administering to the subject a therapeutically effective amount of a (1) a convalescent plasma, wherein the convalescent plasma is collected from (i) a second subject who is recovered from an infection with the SARS-CoV-2 or (ii) a pooled convalescent plasma from a plurality of subjects who are recovered from an infection with the SARS-CoV-2 or (2) purified immunoglobulins (pIVIg) from a SARS-CoV-2 inoculated transgenic animal that produces human immunoglobulins and the pIVIg contains polyclonal human antibodies to SARS-CoV-2. In another embodiment, the provided methods further comprise administering a therapeutically effective amount of a toll-like receptor (TLR) agonist, wherein the TLR agonist is a TLR7 agonist (vesatolimod or imiquimod), and/or a TLR8 agonist (cpd14b or DN052), or a TLR7/8 dual agonist (motolimod (VTX-2337) or selgantolimod (GS-9688)). In a particular embodiment, the TLR7 agonist, TLR8 agonist or a TLR7/8 dual agonist is administered to a male subject.

### Treatment of Non-Covid-19 Viruses

Respiratory viruses (RVs) are an important cause of morbidity and sometimes mortality, some causing outbreaks seasonally, others being prevalent year round. Influenza virus and rhinoviruses are a cause of community-acquired pneumonia, especially in the elderly and children. Adenovirus infections also may result in pneumonia. The present invention provides methods for treating pneumonia and lung injury resulting from non-2019 coronavirus (non-SARS-CoV-2) respiratory viruses, including but not limited to rhinoviruses and adenoviruses.

In one aspect, the present invention provides a method for treating a subject infected with a non-2019 coronavirus respiratory virus (non-SARS-CoV-2), the method comprising administering to the subject a pharmaceutical composition comprising a therapeutically effective amount of a GM-CSF antagonist. In a particular embodiment, the pharmaceutical composition comprises GM-CSF antagonist neutralizing anti-hGM-CSF antibody Lenzilumab. In some embodiments, the GM-CSF antagonist is an anti-GM-CSF antibody selected from the group consisting of Namilumab, Otilimab, Gimsilumab, and TJM2 (TJ003234). In another embodiment, the GM-CSF antagonist is anti-GM-CSF alpha receptor antibody Mavrilimumab. In an embodiment, the pharmaceutical composition is administered at a dose of from 1200 mg to 1800 mg over 24 hours. In certain embodiments, the subject has non-COVID-19 pneumonia, a bacterial respiratory tract infection, a fungal respiratory tract infection. In an embodiment, the provided methods comprise administering an antibacterial agent, an antifungal agent or combinations thereof. In an embodiment of the methods provided herein, the methods further comprise administering a therapeutically effective amount of an anti-viral agent, a steroid, azithromycin, an anti-interleukin-6 (IL-6) receptor monoclonal antibody, an immunoglobulin, intravenous immunoglobulin (IVIG), a statin, and combinations thereof to the subject. In some embodiments, the anti-viral agent comprises Remdesivir (GS-5734), GS-441524, ribavirin, Aribidol (umifenovir), Favilavir, APN01, defensin mimetic Brilacidin, CCR5 antagonist leronlimab (PRO140), Galidesivir (BCX4430), Molnupiravir (MK-4482/EIDD-2801), and MK-7110 (CD24Fc) and combinations thereof and combinations thereof. In another embodiment, the anti-viral agent comprises a combination of fully human neutralizing monoclonal antibodies (mAb) against S-protein of MERS-CoV or the spike protein of SARS-CoV-2, wherein the mAbs comprise REGN3048 and RG3051 or neutralizing monoclonal antibodies against the SARS-CoV-2 spike protein wherein the mAbs comprise REGN-COV2 (casirivimab and imdevimab), BGB-DXP593, CT-P59, VIR-7831, LY-CoV016, and LY-CoV555. In an embodiment, the IVIG comprises human immune globulin g. In some embodiments, the anti-viral agent comprises a combination of antiretroviral drugs, wherein each of the antiretroviral drugs is an inhibitor of HIV-1 protease, or a combination of the inhibitor of HIV-1 protease and a second drug. In an embodiment, the inhibitor of HIV-1 protease is lopinavir or a combination of lopinavir and ritonavir (Lopimune; Aluvia). In some embodiments of the herein provided methods, the combination of the inhibitor of HIV-1 protease and the second drug comprises inhibitor of HIV-1 protease, darunavir, and the second drug is an inhibitor of human CYP3A proteins, wherein the inhibitor of human CYP3A proteins is cobicistat.

In one aspect, the present invention provides a method for improving invasive mechanical ventilator-free survival (VFS) of a subject infected with 2019 coronavirus (SARS-CoV-2) and having COVID-19 pneumonia, the method comprising administering to the subject a therapeutically effective amount of a pharmaceutical composition comprising a hGM-CSF antagonist. In a specific embodiment, the hGM-CSF antagonist is anti-hGM-CSF antibody Lenzilumab. In an embodiment, the hGM-CSF antagonist is administered within one day of hospitalization of the subject. In various embodiments of the therapeutic methods described herein, the anti-hGM-CSF antibody Lenzilumab is administered prior to the subject having respiratory failure and being treated with invasive mechanical ventilation. In some embodiments, improvement of VFS is an improvement compared to an improvement in VFS of a subject treated with placebo. In various embodiments, improvement of VFS is an improvement compared to an improvement in VFS of a subject treated with a steroid and/or antiviral agent remdesivir without the anti-hGM-CSF antibody Lenzilumab. In some embodiments, the method further comprises administering a steroid and/or antiviral agent remdesivir. In an embodiment, In an embodiment, the method further comprises administering low-flow oxygen support. In some embodiments, the method further comprises administering high-flow oxygen support or oxygen via a non-invasive positive pressure device. In certain embodiments, the improvement of VFS comprises a 54% relative increase in chances of the subject surviving and remaining invasive mechanical ventilator (IMV)-free over a time period of 28 days after administration of the anti-hGM-CSF antibody Lenzilumab. In an embodiment, the improvement of VFS comprises the prevention of progression to severe ARDS, respiratory failure, invasive mechanical ventilation and death of the subject. In a particular embodiment, the anti-hGM-CSF antibody Lenzilumab is administered at a dose of from 1200 mg to 1800 mg over 24 hours. In an embodiment, the administered dose is 1,104 mg to 1,656 mg over 24 hours. In some embodiments, the administered dose is 552 mg every eight hours. In certain embodiments, median time to a 2-point clinical improvement on the 8-point hospital ordinal scale of the subject is five days compared to the median time to the 2-point clinical improvement of a subject treated with steroids and/or remdesivir without the anti-hGM-CSF antibody Lenzilumab.

In another aspect, the present invention provides a method for reducing a treatment emergent serious adverse event (TESAE) of a subject infected with 2019 coronavirus (SARS-CoV-2) and having COVID-19 pneumonia, the method comprising administering to the subject a therapeutically effective amount of a pharmaceutical composition comprising a hGM-CSF antagonist. In a particular embodiment, the GM-CSF antagonist is anti-hGM-CSF antibody Lenzilumab. In an embodiment, the GM-CSF antagonist is administered within one day of hospitalization of the subject. In another embodiment, the anti-hGM-CSF antibody Lenzilumab is administered prior to the subject having respiratory failure and being treated with invasive mechanical ventilation. In some embodiments, reduced TESAE is a reduction in the TESAE compared to a reduction in TESAE in a subject treated with placebo and the reduced TESAE is comparable to the TESAE in the subject treated with the placebo. In various embodiments, reduction in TESAE is a reduction in TESAE compared to a reduction in TESAE of a subject treated with a steroid and/or antiviral agent remdesivir without the anti-hGM-CSF antibody Lenzilumab. In some embodiments, the method further comprises administering a steroid and/or antiviral agent remdesivir. In particular embodiments, the method further comprises administering a steroid and/or antiviral agent remdesivir. In an embodiment, the method of claim further comprises administering low-flow oxygen support. In another embodiment, the method further comprises administering high-flow oxygen support or oxygen via a non-invasive positive pressure device. In some embodiments of the provided methods, reduced TESAE prevents progression to severe ARDS, respiratory failure, invasive mechanical ventilation and death of the subject. In a particular embodiment, the anti-hGM-CSF antibody Lenzilumab is administered at a dose of from 1200 mg to 1800 mg over 24 hours. In some embodiments, the administered dose is 1,104 mg to 1,656 mg over 24 hours. In various embodiments, the administered dose is 552 mg every eight hours. In some embodiments median time to a 2-point clinical improvement on the 8-point hospital ordinal scale of the subject is five days compared to the median time to the 2-point clinical improvement of a subject treated with steroids and/or remdesivir without the anti-hGM-CSF antibody Lenzilumab.

In one aspect, the present invention provides a method for treating a subject having emergent COVID-19-associated hyperinflammation (COV-HI), the method comprising administering to the subject a therapeutically effective amount of a pharmaceutical composition comprising an hGM-CSF antagonist. In a particular embodiment, the hGM-CSF antagonist is anti-hGM-CSF antibody Lenzilumab. In some embodiments, the hGM-CSF antagonist is an anti-GM-SCF antibody selected from the group consisting of Namilumab, Otilimab, Gimsilumab, and TJM2 (TJ003234). In another embodiment, the GM-CSF antagonist is anti-GM-CSF receptor antibody Mavrilimumab. In certain embodiments of the herein provided methods, the subject has a baseline serum level of C-reactive protein (CRP) of from greater than 5 mg/L to less than 150 mg/L before administration of the hGM-CSF antagonist. In particular embodiment of the provided methods the subject is from 18 years old to less than 85 years old. In specific embodiments, the subject has a baseline serum level of C-reactive protein (CRP) of from greater than 5 mg/L to less than 150 mg/L before administration of the hGM-CSF antagonist and is from 18 years old to less than 85 years old. In an embodiment, the subject has a baseline serum level of GM-CSF of three or more times higher than 10 pg per milliliter, serum ferritin level >300 ug/L and serum D-dimer level of 500 nanograms per milliliter (mL) or higher. In some embodiments, the subject has severe COVID-19 pneumonia and/or has a pulse oximeter reading (SpO2) ≤94% on room air and/or requires supplemental oxygen. In certain embodiments of the herein provided methods, the methods further comprise administering supplemental oxygen to the subject, wherein the supplemental oxygen is low-flow oxygen, high-flow oxygen, or non-invasive positive pressure ventilation (NPPV). In some embodiments of the herein provided methods, the methods further comprise administering supplemental oxygen to the subject, wherein the supplemental oxygen is low-flow oxygen, high-flow oxygen, or non-invasive positive pressure ventilation (NPPV). In a particular embodiment of the herein provided methods, the subject does not require administration of supplemental oxygen by invasive mechanical ventilator (IMV). In additional embodiments of the herein provided methods, the methods further comprise administering antiviral agent remdesivir and/or a combination of remdesivir and a steroid. In some embodiments, the hGM-CSF antagonist is administered within one to two days of hospitalization of the subject. In an embodiment, the hGM-CSF antagonist is administered within one day of hospitalization of the subject. In certain embodiments, the administration of hGM-CSF antagonist improves survival without ventilation (SWOV) of the subject up to at least 28 days after the administration, improvement of SWOV is an improvement compared to an improvement in SWOV of a subject treated with a placebo. In a particular embodiment, the hGM-CSF antagonist is administered at a dose of from 1200 mg to 1800 mg over 24 hours. In various embodiments of the, the hGM-CSF antagonist is anti-hGM-CSF antibody Lenzilumab. In certain embodiments of the herein provided methods, the hGM-CSF antagonist is an anti-GM-SCF antibody selected from the group consisting of Namilumab, Otilimab, Gimsilumab, and TJM2 (TJ003234). In another embodiment, the GM-CSF antagonist is anti-GM-CSF receptor antibody Mavrilimumab. In some embodiments, the aforementioned hGM-CSF antagonist is administered at a dose of from 1200 mg to 1800 mg over 24 hours. In a particular embodiment, the administered dose of the hGM-CSF antagonist is 1,104 mg to 1,656 mg over 24 hours. In some embodiments, the administered dose of the hGM-CSF antagonist is 552 mg every eight hours.

In another aspect, the present invention provides a method for treating a subject hospitalized with severe COVID-19 pneumonia and having a baseline level of C-reactive protein (CRP) of less than 150 mg/L, the method comprising administering to the subject a therapeutically effective amount of a pharmaceutical composition comprising an hGM-CSF antagonist. In specific embodiments, the hGM-CSF antagonist is anti-hGM-CSF antibody Lenzilumab. In certain embodiments, the hGM-CSF antagonist is an anti-GM-SCF antibody selected from the group consisting of Namilumab, Otilimab, Gimsilumab, and TJM2 (TJ003234). In some embodiments, the GM-CSF antagonist is anti-GM-CSF receptor antibody Mavrilimumab. In particular embodiments of the herein provided methods, the subject has a baseline serum level of C-reactive protein (CRP) of from greater than 5 mg/L to less than 150 mg/L before administration of the hGM-CSF antagonist. In specific embodiments of the provided methods, the subject is from 18 years old to less than 85 years old. In various embodiments, the subject has a baseline serum level of C-reactive protein (CRP) of from greater than 5 mg/L to less than 150 mg/L before administration of the hGM-CSF antagonist and is from 18 years old to less than 85 years old. In an embodiment, the subject has a baseline serum level of GM-CSF of three or more times higher than 10 pg per milliliter, serum ferritin level >300 ug/L and serum D-dimer level of 500 nanograms per milliliter (mL) or higher. In some embodiments, the subject has severe COVID-19 pneumonia and/or has a pulse oximeter reading (SpO2) ≤94% on room air and/or requires supplemental oxygen. In certain embodiments of the herein provided methods, the methods further comprise administering supplemental oxygen to the subject, wherein the supplemental oxygen is low-flow oxygen, high-flow oxygen, or non-invasive positive pressure ventilation (NPPV). In some embodiments of the herein provided methods, the methods further comprise administering supplemental oxygen to the subject, wherein the supplemental oxygen is low-flow oxygen, high-flow oxygen, or non-invasive positive pressure ventilation (NPPV). In a particular embodiment of the herein provided methods, the subject does not require administration of supplemental oxygen by invasive mechanical ventilator (IMV). In additional embodiments of the herein provided methods, the methods further comprise administering antiviral agent remdesivir and/or a combination of remdesivir and a steroid. In some embodiments, the hGM-CSF antagonist is administered within one to two days of hospitalization of the subject. In an embodiment, the hGM-CSF antagonist is administered within one day of hospitalization of the subject. In certain embodiments, the administration of hGM-CSF antagonist improves survival without ventilation (SWOV) of the subject up to at least 28 days after the administration, improvement of SWOV is an improvement compared to an improvement in SWOV of a subject treated with a placebo. In a particular embodiment, the hGM-CSF antagonist is administered at a dose of from 1200 mg to 1800 mg over 24 hours. In various embodiments of the, the hGM-CSF antagonist is anti-hGM-CSF antibody Lenzilumab. In certain embodiments of the herein provided methods, the hGM-CSF antagonist is an anti-GM-SCF antibody selected from the group consisting of Namilumab, Otilimab, Gimsilumab, and TJM2 (TJ003234). In another embodiment, the GM-CSF antagonist is anti-GM-CSF receptor antibody Mavrilimumab. In some embodiments, the aforementioned hGM-CSF antagonist is administered at a dose of from 1200 mg to 1800 mg over 24 hours. In a particular embodiment, the administered dose of the hGM-CSF antagonist is 1,104 mg to 1,656 mg over 24 hours. In some embodiments, the administered dose of the hGM-CSF antagonist is 552 mg every eight hours.

### EXAMPLES

### Example 1

### Preventing and/or Treating Inflammation-Induced Lung Injury Resulting from Coronavirus (SARS-CoV-2) Infection by Administering to an Infected Patient a GM-CSF Antagonist (Lenzilumab)

A patient is diagnosed with SARS-CoV-2 infection and can be considered at high risk of CRS related inflammatory lung injury by having one or more of the following clinical indicators:

Ferritin elevation of >300 mcg/L.
CRP elevation >8 mg/L
Alanine aminotransferase (ALT) elevation that is ten or more times higher than the normal ALT range of 7 to 56 units per liter (U/L).
Aspartate aminotransferase (AST) elevation that is ten or more times higher than the normal AST range of 10 to 40 U/L.
Alkaline phosphatase (ALP) elevation that is ten or more times higher than the normal ALP range of 30 to 130 U/L.
Lactate dehydrogenase (LDH) elevation that is ten or more times higher than the normal LDH range of 140 U/L to 280 U/L.
Creatine kinase (CK) elevation that is ≥3 times greater than upper limits of the normal CK range of 35-175 U/L.
D-dimer elevation that is a level of D-dimer of 500 nanograms per milliliter (mL) or higher.
Prothrombin time (PT) elevation of higher than the upper range of 11 to 13.5 seconds that indicates that it takes blood longer than usual to clot. Conversely, if the PT number is less than the lower range that indicates that blood clots more quickly than normal.
GM-CSF elevation of three or more times higher than 10 pg per milliliter of GM-CSF.
MCP-1 elevation of two or more times higher than 69.5-175.2 pg/mL of MCP-1.
IP10 elevation of ten or more times higher than 41.5 pg/ml of IP10.
MIP1 alpha (also called CCL3) elevation of >10 pg/mL.
IL-6 elevation of 3 times higher than the upper range of 5-15 pg/ml IL-6.
Albumin reduction of below 3.4 grams per deciliter (g/dL).
GM-CSF+CD4+ T cell elevation measured as a percentage of about >3.0% to about 45% of GM-CSF+CD4+ T cells from CD45+CD3+CD4+ T cells isolated from peripheral blood compared to a percentage of about 0% to about 3.0% of GM-CSF+CD4+ T cells from CD45+CD3+CD4+ T cells isolated from peripheral blood of healthy control subjects.
IL-6+CD4+ T cell elevation measured as a percentage of about >1.0% to about 15% of IL-6+CD4+ T cells from CD45+CD3+CD4+ T cells isolated from peripheral blood compared to a percentage of about 0% to about 1.0% of IL-6+CD4+ T cells from CD45+CD3+CD4+ T cells isolated from peripheral blood of healthy control subjects.
INF-γ+GM-CSF+CD4+ T cell elevation measured as a percentage of about >1.0% to about 12.5% of INF-γ+GM-CSF+CD4+ T cells from CD45+CD3+CD4+ T cells isolated from peripheral blood compared to a percentage of about 0% to about 1.0% of INF-γ+GM-CSF+CD4+ T cells from CD45+CD3+CD4+ T cells isolated from peripheral blood of healthy control subjects.
CD14+CD16+ monocyte elevation measured as a percentage of about >10% to about 60% of CD14+CD16+ monocytes from CD45+ monocytes isolated from peripheral blood compared to a percentage of about 0% to 10% of CD14+CD16+ monocytes from CD45+ monocytes isolated from peripheral blood of healthy control subjects.
GM-CSF+CD14+ monocyte elevation measured as a percentage of about >1.25% to about 10% of GM-CSF+CD14+ monocytes from CD14+ monocytes isolated from peripheral blood compared to a percentage of about 0% to about 1.25% of GM-CSF+CD14+ monocytes from CD14+ monocytes isolated from peripheral blood of healthy control subjects.
GM-CSF+CD14+ monocyte elevation measured as a level of about >5×10⁶/L to 35×10⁶/L of GM-CSF+CD14+ monocytes from CD14+ monocytes isolated from peripheral blood compared to a level of about 0×10⁶/L to about 5×10⁶/L of GM-CSF+CD14+ monocytes from CD14+ monocytes isolated from peripheral blood of healthy control subjects.
IL-6+CD14+ monocyte elevation measured as a percentage of about >2.5% to about 20% of IL-6+CD14+ monocytes from CD14+ monocytes isolated from peripheral blood compared to a percentage of about 0% to about 2.5% of IL-6+CD14+ monocytes from CD14+ monocytes isolated from peripheral blood of healthy control subjects.
IL-6+CD14+ monocyte elevation measured as a level of about 10×10⁶/L to 50×10⁶/L of IL-6+CD14+ monocytes from CD14+ monocytes isolated from peripheral blood compared to a level of about 0×10⁶/L to about 9×10⁶/L of IL-6+CD14+ monocytes from CD14+ monocytes isolated from peripheral blood in healthy control subjects.
Hypotension measurement of systolic/diastolic that is less than 90/60 millimeters of mercury (mmHg).
Hypoxemia value of arterial oxygen of under 60 mmHg and/or a pulse oximeter reading of less than or equal to 94% (SpO2≤94%)
Radiological progression of pneumonia shown in chest radiographs as multifocal consolidation, predominantly in the lower lung zone and shown on CT images as ground-glass opacity (GGO), as main findings, and reticulation is noted after the 2nd week. Radiologic findings are usually normal initially or consist of minimal interstitial edema and pleural effusion is common. In some subjects, the radiological findings rapidly progress to bilateral airspace consolidation and fulminant respiratory deterioration within 48 hours.
ARDS (acute respiratory distress syndrome) which is demonstrated radiologically by a diffuse lung damage; a rapidly progressive pneumonia results in ARDS.

A patient displaying one or more of the clinical markers is given a single infusion of lenzilumab at 1,800 mg. In another example, the patient is given three doses of lenzilumab of 600 mg every 8 hours for 24 hours.

In patients receiving lenzilumab, there would be a reduction in patients requiring ICU admission, a reduction in patients requiring mechanical ventilation, and a reduction in mortality rates. In patients receiving lenzilumab, there would be a reduction in the number of hospital days. In patients receiving lenzilumab, there would be a reduction in permanent pulmonary function impairment. In patients receiving lenzilumab there would be faster 2 point improvement in the NIAID eight point ordinal hospital scale and faster time to recovery defined as either a 6, 7, or 8 on the eight point ordinal hospital scale.

Besides the GM-CSF antagonist Lenzilumab, other GM-CSF antagonists that are administered include KB002, mouse neutralizing human GM-CSF antibody LMM102, Mavrilimumab, Namilumab, Otilimab, Gimsilumab, and TJM2 (TJ003234).

### Example 2

### Treating a SARS-CoV-2 Infected Patient with a GM-CSF Antagonist or in Combination with an Anti-Viral Agent(s)

A patient is diagnosed with SARS-CoV-2 infection and can be considered at high risk of CRS related inflammatory lung injury by having and elevated Ferritin level (>300 ug/L). A patient displaying an elevated Ferritin level is given an infusion of lenzilumab at 600 mg Q8 hours for three doses.

In patients receiving lenzilumab, there would be a reduction in patients requiring ICU admission, a reduction in patients requiring mechanical ventilation, and a reduction in mortality rates. In patients receiving lenzilumab, there would be a reduction in the number of hospital days. In patients receiving lenzilumab, there would be a reduction in permanent pulmonary function impairment. In patients receiving lenzilumab there would be faster 2 point improvement in the NIAID eight point ordinal hospital scale and faster time to recovery defined as either a 6, 7, or 8 on the eight point ordinal hospital scale.

Besides the GM-CSF antagonist Lenzilumab, other GM-CSF antagonists that are administered include KB002, or mouse neutralizing human GM-CSF antibody LMM102, Mavrilimumab, Namilumab, Otilimab, Gimsilumab, and TJM2 (TJ003234).

### Example 3

### Preventing and/or Treating Inflammation-Induced Lung Injury Resulting from Coronavirus (SARS-CoV-2) Infection by Administering to a Patient a GM-CSF Antagonist in Combination with an Anti-Viral Agent(s)

A patient is diagnosed with SARS-CoV-2 and is deemed to be at high risk of CRS related inflammatory lung injury, as following the procedures described in Example 1. The patient displaying one or more of the clinical markers is administered an antiviral therapy as a sequenced therapy in combination with lenzilumab: a single infusion of lenzilumab at 1,800 mg and 200 mg of Remdesivir (anti-viral agent) on day 1. Remdesivir is then dosed daily at 100 mg/kg for 10 days.

Additional (or alternate) anti-viral agents/drugs that are administered are selected from the following anti-viral agents/drugs or combinations thereof: Aribidol (umifenovir), Favilavir, APN01, Brilacidin (a defensin mimetic), leronlimab (CCR5 antagonist), Remdesivir (GS-5734), GS-441524, Galidesivir (BCX4430), Molnupiravir (MK-4482/EIDD-2801), and MK-7110 (CD24Fc), REGN3048 plus RG3051 (antibodies to the S-protein of MERS virus), antibodies to the S-protein of SARS-CoV-2 virus (REGN-COV2, LY-CoV555), Lopinavir, a combination of Lopinavir and ritonavir (Lopimune; Aluvia) and combinations thereof.

The herein provided combination therapy is expected to reduce the number patients requiring ICU admission, reduction the number patients requiring mechanical ventilation, and reduce in mortality rates in patients infected with SARS-CoV-2. In patients receiving lenzilumab, there is expected to be a reduction in the number of hospital days. In patients receiving lenzilumab, there is expected to be a reduction in permanent pulmonary function impairment. In patients receiving lenzilumab there would be faster 2 point improvement in the NIAID eight point ordinal hospital scale and faster time to recovery defined as either a 6, 7, or 8 on the eight point ordinal hospital scale.

### Example 4

### Preventing and/or Treating Inflammation-Induced Lung Injury Resulting from Coronavirus (SARS-CoV-2) Infection by Administering to an Infected Patient a Combination Therapy of a GM-CSF Antagonist and an Anti-Viral Agent(s)

A patient is diagnosed with SARS-CoV-2 and is deemed to be at high risk of CRS related inflammatory lung injury, as following the procedures described in Example 1. The patient displaying one or more of the clinical markers is treated by administration of an antiviral therapy as a sequenced therapy in combination with lenzilumab, as described in Example 2. The patient also is administered an IL-6 antagonist (tocilizumab), as a sequenced therapy with lenzilumab and the antiviral therapy.

The herein provided combination therapy is expected to reduce the number patients requiring ICU admission, reduction the number patients requiring mechanical ventilation, and reduce in mortality rates in patients infected with SARS-CoV-2. In patients receiving lenzilumab there would be faster 2 point improvement in the NIAID eight point ordinal hospital scale.

### Example 5

### Combination Therapy Comprising a GM-CSF Antagonist and an Anti-Viral Agent(s) for Preventing and/or Treating Inflammation-Induced Lung Injury Resulting from Coronavirus (SARS-CoV-2) Infection

A patient is diagnosed with SARS-CoV-2 and is deemed to be at high risk of CRS related inflammatory lung injury, following the procedures described in Example 1. The patient displaying one or more of the clinical markers is treated by administration of lenzilumab (600 mg) every three days for 9 days and 100 mg Remdesivir (anti-viral agent) daily for 10 days.

The herein provided combination therapy is expected to reduce the number patients requiring ICU admission, reduction the number patients requiring mechanical ventilation, and reduce in mortality rates in patients infected with SARS-CoV-2. In patients receiving lenzilumab there would be faster 2 point improvement in the NIAID eight point ordinal hospital scale.

### Example 6

### Combination Therapy Comprising a GM-CSF Antagonist and Anti-Viral Agents for Preventing and/or Treating Inflammation-Induced Lung Injury Resulting from Coronavirus (SARS-CoV-2) Infection

A patient is diagnosed with SARS-CoV-2 and considered at high risk of CRS related inflammatory lung injury, as described in Example 1, and is dosed with Lenzilumab (600 mg) every three days for 9 days and REGN3048 (600 mg) plus RG3051 (600 mg) on days 1 and 3.

The herein provided combination therapy is expected to reduce the number patients requiring ICU admission, reduction the number patients requiring mechanical ventilation, and reduce in mortality rates in patients infected with SARS-CoV-2. In patients receiving lenzilumab there would be faster 2 point improvement in the NIAID eight point ordinal hospital scale.

### Example 7

### Combination Therapy Comprising a GM-CSF Antagonist and an Anti-SARS-CoV-2 S Protein Antibody for Preventing and/or Treating Inflammation-Induced Lung Injury Resulting from Coronavirus (SARS-CoV-2) Infection

A patient is diagnosed with SARS-CoV-2 and considered at high risk of CRS related inflammatory lung injury, as described in Example 1, and is dosed with Lenzilumab (600 mg) every three days for 9 days and 1800 mg of an anti-SARS-CoV-2 S protein antibody (as described hereinabove) on day 1.

The herein provided combination therapy is expected to reduce the number patients requiring ICU admission, reduction the number patients requiring mechanical ventilation, and reduce in mortality rates in patients infected with SARS-CoV-2. In patients receiving lenzilumab there would be faster 2 point improvement in the NIAID eight point ordinal hospital scale.

### Example 8

### First Cases of COVID-19 Patients Treated with Lenzilumab on a Compassionate Use-Basis Patient 1

Given the hypothesized role of GM-CSF in the pathogenesis of COVID-19 related CRS, along with our studies demonstrating that GM-CSF depletion prevents CRS and modulates myeloid cell behavior in preclinical models, lenzilumab therapy was offered to patients hospitalized with severe COVID-19 pneumonia, who had clinical and/or biomarker evidence (e.g., inflammatory markers) for increased risk of progression to respiratory failure.

### Methods

### Patients

Hospitalized patients with COVID-19, confirmed by reverse transcriptase-polymerase chain reaction for the SARS-CoV-2, and radiographic findings consistent with COVID-19 pneumonia were considered for treatment with lenzilumab through an emergency IND program. Active systemic infection with bacteria, fungi, or other viruses, was an exclusion criterion. All patients received lenzilumab 600 mg administered via a 1-hour intravenous infusion every 8 hours for a total of three doses (1800 mg). A request for lenzilumab under FDA emergency use IND was submitted to the FDA in accordance with agency guidelines (www.fda.gov/regulatory-information/search-fda-guidance-documents/emergency-use-investigational-drug-or-biologic). Informed consent and Institutional review board approval was obtained for each patient.

### Patient 1

Patient 1 is a 29-year-old woman with obesity (BMI 30) who was admitted on Apr. 6, 2020. Patient had developed fever, dry cough, generalized weakness and body aches on March 30. On April 1, nasopharyngeal swab was positive for SARS-CoV-2 by real-time reverse transcription polymerase chain reaction (PCR) assay via drive-through testing. She subsequently developed dyspnea on exertion, diarrhea, nausea and anorexia on April 5, prompting presentation to the emergency room and ICU admission on April 6. She was previously healthy, with recent exposure to a laboratory-confirmed COVID-19 case. On admission, her temperature was 38.2° C., blood pressure 134/93, pulse 112, respiratory rate 16, and oxygen saturation 97% on room air at rest, 84% on exertion. Both lungs were clear on auscultation, and the remainder of the physical examination was unremarkable. Laboratory evaluation revealed an elevated C reactive protein (CRP) 100 mg/L (Table 4), with normal complete blood count (CBC), liver function and renal function. (Table 4) Chest CT showed patchy bilateral ground-glass and consolidative opacities predominantly in the peripheries. Supportive care and empiric ceftriaxone and azithromycin were started, with close monitoring of clinical status. On April 7, the patient was requiring 2 L/min of oxygen via nasal cannula to maintain an oxygen saturation of 92%. She remained afebrile with stable vitals. She received Lenzilumab on that day at 600 mg administered as intravenous infusions every 8 hours for total of 3 doses. On April 8, the patient was weaned off of supplemental oxygen, maintaining oxygen saturations between 90-99% on room air. Antibiotics were discontinued. CRP had decreased to 91, with further decrease to 46 on April 9. (Table 4) Patient was discharged home on that day. Table 2 shows the patient's CBC Lab results, for neutrophils and lymphocytes, including first, high and last results after administration of Lenzilumab. On outpatient follow-up on April 11 via telephone, the patient stated that she feels much better; she has some residual cough but no fevers or headache.

### Patient 2

Patient 2 is a 62-year-old female who was admitted to the hospital on Apr. 1, 2020. She had a history of end-stage renal disease secondary to diabetic nephropathy status post living donor kidney transplant in 2005, hypertension, congestive heart failure and obstructive sleep apnea on CPAP. She was on chronic immunosuppression with tacrolimus 3 mg twice daily and mycophenolate mofetil 750 mg twice daily. She first developed fevers, nasal congestion and cough around 2 weeks prior to admission, with progressive shortness of breath, myalgias, fatigue and anorexia over the week leading to her admission. Her husband died on March 29 from severe COVID-19 pneumonia after returning from a trip to California.

On admission, she was afebrile, blood pressure was 145/105, pulse 72, respiratory rate 22, and her oxygen saturation was 80% on room air requiring 3 L/min oxygen via nasal cannula. She had decreased breath sounds bilaterally at the bases with bilateral lower extremity edema. Laboratory evaluation revealed a white blood cell count of 4.4×10⁹/L and lymphopenia with an absolute lymphocyte count of 0.62×10⁹/L. (Table 4) She had acute kidney injury with a creatinine of 2.1 mg/dL increased from a previous baseline of 1.7 mg/dL. Troponin T was elevated at 71 ng/L; however, this was lower than her recent baseline of 571. Liver function tests were within normal range. Chest x-ray showed stable chronic bilateral moderate pleural effusions and bibasilar consolidations, with a new left upper lobe consolidation (img 4/1). Nasopharyngeal swab was positive for SARS-CoV-2 by RT-PCR. She received one dose of empiric cefepime, which was discontinued after this result Inflammatory markers were obtained on April 3 (hospital day 2) and were found to be elevated, with CRP of 29.7 mg/L, serum ferritin of 548 mcg/L, D-dimer 1,537 ng/mL and interleukin 6 (IL-6) level of 34.7 pg/mL. (Table 4) Meanwhile, the patient's clinical status remained largely stable with supportive care only, including gentle diuresis and reduction of immunosuppression by switching mycophenolate mofetil to prednisone 10 mg daily. On April 6 (hospital day 5), the patient developed increased respiratory distress with worsening hypoxemia requiring high-flow oxygen at 30 L/min and 50% FiO2, culminating in transfer to the intensive care unit (ICU). Repeat chest x-ray showed interval progression with new foci of airspace opacities in the left upper lung as well as in the bilateral perihilar regions, with persistent moderate-large bilateral pleural effusions (img 4/6). This was accompanied by an increasing CRP, reaching its peak on that day at 41.2 mg/L. (Table 4) Other inflammatory markers were also persistently elevated with ferritin 621 mcg/L, lactate dehydrogenase (LDH) 283 U/L, and IL-6 26.2 pg/mL. D-dimer peaked at 1759 ng/mL on April 7, and ferritin peaked at 1143 mcg/L on April 9. (Table 4) The patient received lenzilumab on April 6 through April 7 at 600 mg administered as intravenous infusions every 8 hours for total of 3 doses. Of note, the patient did develop a transient exacerbation of her restless leg syndrome 20 minutes into her first lenzilumab infusion. On April 7, the patient remained with a stable oxygen requirement, albeit without improvement. Chest x-ray on April 7 showed continued progression of airspace disease with near complete opacification of the bilateral lungs (img 4/7). Her pleural effusions were drained, which yielded transudative fluid. On April 8, her CRP and D-dimer improved to 22.4 mg/L and 1507 ng/mL, respectively. (Table 4) However, she continued to have increasing hypoxemia; repeat chest x-ray revealed bilateral pneumothoraces (img 4/8). Her respiratory status subsequently improved with pleural drain management, and she was weaned off of supplemental oxygen by April 14 (img 4/14). However, she continued to require 2 L of oxygen via nasal cannula intermittently throughout the rest of her hospital stay. Her discharge was delayed due to social issues, but she was finally discharged on April 25 on 2 L of oxygen via nasal cannula. Table 2 shows the patient's CBC Lab results, for neutrophils and lymphocytes, including first, high and last results after administration of Lenzilumab.

### Patient 3

Patient 3 is a 38-year-old male, who was admitted on April 5. He is a former smoker with a history of latent tuberculosis treated with isoniazid in 2010, otherwise healthy. On March 29, he developed fevers, myalgias, sore throat, headache, anosmia, nausea, vomiting and diarrhea. Nasopharyngeal swab was positive for SARS-CoV-2 by RT-PCR on March 31 via drive-through testing. He presented to the emergency room on April 5 with increasing shortness of breath and chest tightness. He was afebrile, blood pressure 106/73, pulse 82, respiratory rate 30, and oxygen saturation 99% on 2 L of oxygen via nasal cannula. The patient was uncomfortable and had increased work of breathing with clear lungs bilaterally. On laboratory evaluation, D-dimer and ferritin were elevated at 951 ng/mL and 356 mcg/L, respectively, but CRP was less than 3 mg/L. (Table 4) Chest x-ray revealed no abnormalities, however, chest CT with angiography showed scattered patchy peripheral ground-glass opacities in the bilateral lower lobes, without evidence of pulmonary embolism (img 4/5). EKG and troponin T were normal. The patient was admitted and started on hydroxychloroquine on April 6 (hospital day 1) at 400 mg orally twice daily followed by 200 mg twice daily. Lenzilumab was given infusions in 3 doses of 600 mg administered 8 hours apart on April 6. By April 7 (hospital day 2), the patient's respiratory symptoms had improved, and he remained afebrile. However, he noted isolated worsening of his diarrhea and nausea. Hydroxychloroquine was discontinued on April 8 (hospital day 3) due to diarrhea. On discharge on April 8, his ferritin had increased to 571 mcg/L, however, his CRP remained low. A repeat D-dimer was not obtained. Table 2 shows the patient's CBC Lab results, for neutrophils and lymphocytes, including first, high and last results after administration of Lenzilumab. On outpatient follow up on April 10 via telephone, dyspnea, chest tightness and cough, have continued to improve. Patient presented to the emergency room on April 22 with bilateral jaw pain radiating to the ears, associated with tinnitus and epigastric pain. His cough and shortness of breath were still present since diagnosis with COVID-19, though improved. His temperature was 36.6° C., heart rate 88, respiratory rate 16, blood pressure 107/67 and oxygen saturation was 97% on room air. EKG was normal, and chest x-ray did not show any new infiltrates (img 4/22). On routine repeat SARS-CoV-2 PCR testing on April 19 per institutional protocol, the patient had still tested positive. The patient was noted to have anterior temporomandibular joint dislocation bilaterally on physical examination. He was reassured and discharged home. No subsequent follow up documented.

### Patient 4

Patient 4 is a 68-year-old man with hypertension and obstructive sleep apnea on nocturnal CPAP, admitted on April 5. On March 31, he developed fever, cough, shortness of breath, nasal congestion and malaise, progressing with increased chest pain prompting presentation to the emergency department. On admission, his temperature was 38.4° C., blood pressure 141/74, pulse 84, respiratory rate 26 and oxygen saturation 89% on room air and 92% on 4 L of oxygen via nasal cannula. The patient had increased work of breathing with inability to complete sentences, and bilateral crackles at the lung bases. Laboratory evaluation showed mild thrombocytopenia. Alkaline phosphatase was elevated at 205 U/L; liver function tests were otherwise normal as was his renal function. CRP (61.2 mg/L), D-dimer (571 ng/mL), LDH (282 U/L), ferritin (519 mcg/L) and IL-6 (27.1 pg/mL) were elevated. (Table 4) EKG and troponin T were unremarkable. Chest x-ray showed low lung volumes and bilateral lower lobe predominant parenchymal opacities (img 4/5). Nasopharyngeal swab was positive for SARS-CoV-2 by RT-PCR. He received lenzilumab administered as three 600 mg infusions separated by 8 hours starting on April 5 through April 6. He was concomitantly started on a five-day course of hydroxychloroquine administered at 300 mg twice on day 1 followed by 200 mg twice daily, which he completed on April 10 (hospital day 5). Despite this, however, the patient's clinical status progressively worsened with ongoing fevers and worsening hypoxia 15 L non-rebreather mask, proning and transfer to the ICU on April 8 (hospital day 3), where he was initiated on high-flow nasal cannula via helmet. Repeat chest x-ray showed worsening airspace disease (img 4/8), and inflammatory markers continued to increase: CRP 175.8 mg/L, D-dimer 1802 ng/mL, IL-6 95.4 pg/mL, and LDH 388 U/L. (Table 4) In light of worsening clinical condition, with markedly elevated IL-6, the patient received a dose of tocilizumab off-label on April 11 (hospital day 6), as well empiric cefepime and azithromycin for possible superimposed bacterial pneumonia for a total of 5 days. His inflammatory markers and oxygen requirements progressively improved and he was discharged home on 2 L of oxygen via nasal cannula on April 18. Of note, he did develop a transient elevation in his liver enzymes during his hospital stay starting on April 9, with ALT (alanine aminotransferase) peaking at 169 and AST (aspartate aminotransferase) peaking at 203 on April 14 with subsequent improvement. Also, of note, the patient did receive full-dose heparin while in the ICU due to elevated D-dimer and high risk for venous thromboembolism. Table 2 shows the patient's CBC Lab results, for neutrophils and lymphocytes, including first, high and last results after administration of Lenzilumab. On outpatient follow-up on April 23 via telephone, the patient reported continued improvement in his fatigue and shortness of breath, and he remained on 2 L of oxygen via nasal cannula with oxygen saturation at 90%.

### Patient 5

Patient 5 is a 55-year-old man with mild reactive airway disease, who was admitted on March 24. He initially presented to the emergency room on March 17 with fever, cough, nasal congestion, myalgias and fatigue. Nasopharyngeal swab was positive for SARS-CoV-2 by RT-PCR on March 17. In the emergency room, temperature was 38.5° C., blood pressure 154/85, pulse 75, respiratory rate 20, oxygen saturation 98% on room air. Chest x-ray was unremarkable. Given his clinical stability, he was discharged home to quarantine; however, his symptoms progressed with ongoing fevers and increased shortness of breath and anorexia, prompting return to the emergency room and ICU admission on March 24. On admission, temperature was 39.1° C., blood pressure 139/79, pulse 85, respiratory rate 23, oxygen saturation 89% on room air requiring 2 L oxygen via nasal cannula. His lungs were clear bilaterally. Laboratory evaluation showed lymphopenia and mild abnormalities in AST and ALT, 72 and 41, respectively. CRP was elevated at 53.4; other inflammatory markers were not obtained. Chest x-ray showed new bilateral patchy opacities with peripheral and basal predominance, consistent with COVID-19 pneumonia (img 3/24). On March 25, the patient had been weaned off of supplemental oxygen and was transferred out of the ICU to the general floor. However, he continued to have intermittent fevers and he developed recurrent hypoxemia requiring 2 L of oxygen. Repeat chest x-ray on March 26 showed increased patchy airspace opacities (img 3/26). In light of his clinical and radiographic worsening, the patient was started on remdesivir (RDV) in the context of a clinical trial, which he received for total of 5 days at a dose of 200 mg on Day 1 followed by RDV 100 mg on Days 2, 3, 4, and 5. He also completed a 5 day course of ceftriaxone for possible superimposed bacterial pneumonia. However, he continued to have intermittent fevers and remained on 2 L of oxygen. One week into his hospitalization, on March 31, his oxygen requirement increased to 4 L. Repeat chest x-ray showed worsening diffuse patchy airspace opacities (img 3/31). Laboratory evaluation revealed a new leukocytosis with a white blood cell count of 11.7 with left shift, and a near 3-fold increase from baseline CRP to 184.4. (Table 4) Ferritin and IL-6 were also elevated at 1269 and 23.2, respectively. (Table 4) Meanwhile, liver enzymes had continued to increase, with AST and ALT now at 101 and 98, respectively. Lenzilumab was given on April 2 as 3 infusions of 600 mg separated by 8 hours on April 2. He experienced clinical improvement on the following day with resolution of fevers and improvement in his supplemental oxygen requirement to 2 L. On April 5, the patient was discharged home on new supplemental oxygen therapy. Inflammatory markers had improved, down to CRP 22, ferritin 1223, and IL-6 4.6. Liver function tests had also improved to AST 96 and ALT 175, after peaking at 182 and 190, respectively. Table 2 shows the patient's CBC Lab results, for neutrophils and lymphocytes, including first, high and last results after administration of Lenzilumab. On outpatient follow-up on April 9 via telephone, the patient reported continued improvement and stated that he had not required any oxygen therapy for the past 2 days.

None of the above-described five patients required mechanical invasive ventilation.

### Patient 6

Patient 6 is a 75-year-old male with type 2 diabetes mellitus and chronic obstructive pulmonary disease (COPD) on chronic oxygen therapy, admitted on April 6. He developed fever, cough, shortness of breath and fatigue on April 3. He presented to an urgent care clinic on April 6 where he was found to have an oxygen requirement of 3 L of oxygen, increased from a baseline of 2 L. On admission, his temperature was 36.3° C., pulse 70, respiratory rate 20, blood pressure 110/70 and oxygen saturation 88% on 2 L of oxygen. Decreased air movement was noted on auscultation of both lungs. Chest x-ray did not show any infiltrates (img 4/6). Laboratory evaluation revealed lymphopenia. Nasopharyngeal swab was positive for SARS-CoV-2 by RT-PCR. He was diagnosed with COPD exacerbation in the setting of COVID-19 infection. Following admission, his hypoxemia continued to progress, requiring up to 15 L of oxygen via high-flow nasal cannula on April 8. He received hydroxychloroquine for a total of 10 days. He also received a 5 day course of ceftriaxone and a 7 day course of doxycycline to empirically cover for possible community-acquired pneumonia. Repeat chest x-ray on April 11 showed peripherally predominant bilateral infiltrates (img 4/11). Inflammatory markers were obtained on April 10 and were elevated with ferritin 968, CRP 253.4 and interleukin 643.5. These were repeated on April 15, prior to receiving lenzilumab, and were persistently elevated with ferritin 709, CRP 109.7, interleukin 6 20.8 and D-dimer 829. The patient then received lenzilumab from April 15 through April 16 as 3 infusions of 600 mg separated by 8 hours. His inflammatory markers subsequently improved, with slow improvement in his oxygen requirements. At the time of discharge on April 21, he was on 4 L of oxygen via nasal cannula. On outpatient follow-up on April 24 via telephone, he was reportedly feeling better and his oxygen saturation was 91% on 3 L of oxygen via nasal cannula. Of note, workup for alternative diagnoses was conducted during his hospital stay with a nasopharyngeal swab for influenza A/B and respiratory syncytial virus PCR, urine Legionella antigen, urine Streptococcus pneumoniae antigen and blood cultures. This workup was nonrevealing. Also, of note, the patient did not receive any steroids for his COPD exacerbation due to concerns for potential worsening of COVID-19 pneumonia with steroid therapy.

### Patient 7

Patient 7 is a 69-year-old man with obesity (BMI 36), type 2 diabetes and hypertension who was admitted on April 14. He first developed cough, sore throat and myalgia on April 5. This progressed to shortness of breath, chest tightness, fever, green sputum production, nausea and diarrhea beginning April 10. He presented to a local hospital on April 13 due to worsening shortness of breath. He was found to be hypoxemic to 86% on room air, requiring 3 L of oxygen. Laboratory evaluation was notable for lymphopenia and CRP 168.7. Chest x-ray and CT of the chest with contrast angiography demonstrated bilateral multifocal ground-glass infiltrates, without evidence of pulmonary embolism (4/13). On transfer to our facility on April 14, his temperature was 36.3° C., pulse 84, respiratory rate 18, blood pressure 125/69 and oxygen saturation 92% on 1 L of oxygen. Faint rales were noted on auscultation of bilateral mid and lower lung zones. Nasopharyngeal swab was positive for SARS-CoV-2 by RT-PCR. Further laboratory evaluation was notable for elevated inflammatory markers with a markedly elevated D-dimer of 12,160, CRP 154.5 and ferritin 365. Fibrinogen was also elevated at 795, raising concerns for high risk for thromboembolism and prompting initiation of intermediate dose anticoagulation with 0.5 milligram/kilogram of enoxaparin twice daily. INR and aPTT were at 189 1.1 and 27, respectively, with a normal platelet count. There were no renal or liver function test abnormalities. Procalcitonin was elevated at 0.16. The patient was empirically started on ceftriaxone for possible bacterial community-acquired pneumonia. On April 15, the patient was febrile to 39.3° C., and he continued to have fluctuating oxygen saturations, intermittently fluctuating between room air and 2 L of oxygen. He received lenzilumab on April 16 through 17 as 3 infusions of 600 mg separated by 8 hours, with subsequent improvement in his oxygen requirement and inflammatory markers. He was discharged home on April 20 on 1 L of nocturnal oxygen. He was lost to follow-up after discharge.

### Patient 8

Patient 8 is a 41 year old male with obesity (BMI 35) admitted on April 18. He is also an ex-200 smoker, who quit smoking in 2015, and continues to vape. He developed fever, chest pain, cough and anorexia on April 13. Nasopharyngeal swab was positive for SARS-CoV-2 by RT-PCR on April 14 via drive through testing. He presented to the emergency room on April 18 with worsening shortness of breath. On admission his temperature was 39° C., blood pressure 116/100, pulse 115, respiratory rate 22 and oxygen saturation 95% on room air. Chest x-ray was unremarkable (img 4/17). Laboratory evaluation was notable for mild transaminitis with ALT 167 and AST 117, as well as elevated inflammatory markers. EKG showed sinus tachycardia and troponin T was normal. Over the first couple of days following admission, he developed hypoxemia requiring up to 4 L of oxygen. Repeat chest x-ray demonstrated interval development of bilateral interstitial infiltrates (img 4/19, 4/20 and 4/21). He remained febrile and his inflammatory markers continued to rise, prompting administration of lenzilumab on April 21 as 3 infusions of 600 mg separated by 8 hours. His inflammatory markers remained stable over the next couple of days, albeit still elevated. His liver enzymes also remained mildly elevated with ALT 137 and AST 118 at the time of discharge on April 23. His oxygen requirement had meanwhile improved, though not back to baseline. He was discharged on 2 L of oxygen via nasal cannula. No follow-up documented to date.

### Patient 9

Patient 9 is a 81 year old male with a history of prostate status post chemotherapy and androgen deprivation therapy in 2013, chronic kidney disease stage 3 and osteopenia, who was admitted on April 21. He initially developed fatigue, myalgias, anosmia and diarrhea on April 14. Nasopharyngeal swab was positive for SARS-CoV-2 by RT-PCR on April 15 via drive through testing. He subsequently developed sore throat, dry cough, anorexia, nausea and worsening fatigue and shortness of breath prompting presentation to the emergency room and admission to the ICU on April 21. On admission, his temperature was 37.4° C., heart rate 68, blood pressure 155/70, respiratory rate 27 and oxygen saturation 88% on 6 L of oxygen via nasal cannula requiring 100% non-rebreather mask. He was transitioned to high-flow nasal cannula at 15 liters/minute and FiO2 100%, which was titrated down to 80% within a few hours. EKG was normal. BNP was elevated at 5030 as was troponin T at 80, though the latter did not increase when trended. Laboratory evaluation revealed mild leukocytosis with a white blood cell count of 11,200 with left shift and relative lymphopenia, and acute on chronic renal failure with a BUN of 97, bicarbonate of 17, potassium of 5 and serum creatinine of 7 from a baseline creatinine 1.4-1.6 (creatinine clearance<15). Urinalysis revealed renal tubular epithelial cells consistent with acute tubular necrosis and a nephrotic range proteinuria Inflammatory markers were markedly elevated. Chest x-ray showed bilateral ground-glass infiltrates (img 4/21). He received lenzilumab on April 22 as 3 infusions of 600 mg separated by 8 hours. He was concomitantly started on steroid therapy as part of a clinical trial, ultimately completing a 5 day course of steroids. On April 23, he was started on low-intensity heparin in light of his extremely elevated D-dimer and thus high risk for venous thromboembolism. Of note, he also had an exceedingly elevated soluble fibrin monomer (exceeding 1100) with normal prothrombin time, platelet count, fibrinogen and coagulation factor levels, raising suspicion for compensated DIC. Heparin was temporarily held on April 24 for a kidney biopsy done to investigate nephrotic range proteinuria, which showed membranous nephropathy in addition to acute tubular necrosis. Despite improvement in his inflammatory markers, his oxygen requirement continued to increase. On April 27, the patient acutely decompensated with worsening hypoxemia, increased work of breathing and increased sputum production requiring intubation and mechanical ventilation. This decompensation was associated with hypotension requiring 3 vasopressors. Chest x-ray did not reveal any progression in pulmonary infiltrates or any other new findings. He was started on broad-spectrum antibiotics with vancomycin and cefepime for suspected superimposed bacterial pneumonia. On April 28, he was found to have a profound drop and a kidney ultrasound revealed a subcapsular perinephric hematoma, for which he received a transfusion with 1 unit of packed red blood cells.

He was weaned off of pressors on April 29, however, he continued to have progressive hypoxemia and was proned, with subsequent improvement in oxygenation and resumption of supine position by April 30. CT of the abdomen and pelvis showed no progression of his known perinephric hematoma and no evidence of active extravasation. CT of the chest showed diffuse mid and lower lung predominant ground-glass and micronodular opacities with bibasilar consolidation consistent with COVID-19 pneumonia. There was no radiologic evidence of superimposed bacterial pneumonia, and cultures from tracheal secretions grew usual flora. Vancomycin was stopped, and cefepime was switched to piperacillin-tazobactam to complete a total of 5 days of antibiotic therapy. On May 1, he continues to require paralytics to maintain adequate oxygenation on the ventilator, however, indicating severely impaired lung compliance.

### Patient 10

Patient 10 is a 59-year-old female with a history of diabetes mellitus, hypertension (HTN), obesity (BMI 37), obstructive sleep apnea not on CPAP and migraine headache disorder, who was admitted on April 20. She initially developed sore throat, myalgias, chest pain, shortness of breath and diarrhea on April 11. Nasopharyngeal swab was positive for SARS-CoV-2 by RT-PCR on April 14 via drive through testing. Her symptoms subsequently progressed with worsening shortness of breath, chest pain, diarrhea, headache and nausea prompting presentation to the emergency room and admission on April 20. On admission, her temperature was 35.8 degree C., heart rate 106, respiratory rate 22, blood pressure 118/85 and oxygen saturation 90% on room air. Laboratory evaluation revealed leukopenia with lymphopenia. EKG was notable for sinus tachycardia. Chest x-ray and CT showed bilateral multifocal ground-glass opacities (img 4/20). On April 21, she developed respiratory distress with increased work of breathing and oxygen desaturation to 89% on 5 L of oxygen via face mask. She was initiated on BiPAP and transferred to the ICU Inflammatory markers were elevated with CRP 31.4, ferritin 111, D-276 dimer 457 and interleukin 82.8. On April 22, she received lenzilumab as 3 infusions of 600 mg separated by 8 hours. She subsequently improved and was transferred out of the ICU on 3 L of oxygen via nasal cannula on April 24, with improvement in her CRP and IL-6. She was weaned completely off of supplemental oxygen by April 28 and was discharged home on that day.

### Patient 11

Patient 11 is a 73 year old man who is a nursing home resident with type 2 diabetes and history of traumatic brain injury, admitted on April 22. He was brought to the emergency department from his nursing home with confusion, shortness of breath and cough of a few days' duration. Nasopharyngeal swab was positive for SARS-CoV-2 by RT-PCR on April 20. On admission, his temperature was 38.4 degrees C., heart rate 110 beats per minute, respiratory rate 52 breaths per minute, blood pressure 131/93 and oxygen saturation 88% on room air requiring 4 L of oxygen via nasal cannula to maintain an oxygen saturation of 95%. Laboratory evaluation was notable for lymphopenia and thrombocytopenia. Renal and liver function tests were within normal limits. Inflammatory markers were elevated. Chest x-ray showed patchy airspace opacities in the left mid and lower lung fields. He received lenzilumab on April 22 through April 23 as 3 infusions of 600 mg separated by 8 hours. By April 23, he had been weaned down to 1 L of oxygen via nasal cannula, and was weaned off of supplement oxygen completely by April 27. He remained afebrile and his inflammatory markers improved, as did his thrombocytopenia. He was discharged back to his nursing home on April 29 in stable condition and on room air.

### Patient 12

Patient 12 is a 68-year-old woman with coronary artery disease, congestive heart failure, hypertension, atrial fibrillation, type 2 diabetes, obesity, obstructive sleep apnea on CPAP, COPD and prior smoking history who was admitted on April 26. She initially developed sore throat, cough, myalgia, pleuritic chest pain, abdominal pain and diarrhea on April 14. Nasopharyngeal swab was positive for SARS-CoV-2 by RT-PCR on April 15. She subsequently had increased shortness of breath prompting presentation to the emergency room and admission on April 16. In light of her clinical stability, lack of hypoxemia and lack of chest x-ray abnormalities, she was managed conservatively with subsequent symptom improvement and discharge home on April 19. However, on April 25, she again developed worsening symptoms, this time accompanied by fever and hypoxemia with oxygen saturation 85% on room air. She thus presented again to the emergency room and was readmitted on April 26. On admission, her temperature was 38.4 degree C., heart rate 78 beats per minute, respiratory rate 23 breaths per minute, blood pressure 129/67 and oxygen saturation 91% on 3 L of oxygen via nasal cannula. She appeared to have increased work of breathing on physical examination, with decreased air movement and wheezes on auscultation of bilateral lungs. Laboratory evaluation revealed acute kidney injury, lymphopenia, and hyponatremia. Liver function tests were within normal range. There was no increase in her chronically elevated troponin T levels, and EKG showed no acute abnormalities. Chest x-ray showed new multifocal peripheral ground-glass opacities. These findings were re-demonstrated on chest CT with angiography, which did not show evidence of pulmonary embolism. CT of the abdomen and pelvis showed no acute intra-abdominal findings. Patient 12 had elevations in inflammatory markers CRP, Ferritin, IL-6, and D-dimer. She received lenzilumab on April 26 through April 27 as 3 infusions of 600 mg separated by 8 hours. She did experience chills with lenzilumab infusions, but otherwise experienced no complications. The patient progressively improved in terms of her symptoms, fevers and kidney function. However, she continued to require continuous supplemental oxygen with nocturnal bilevel positive pressure ventilation. She was discharged home on April 29 on 2 L of oxygen via nasal cannula. Of note, the patient had also been empirically started on ceftriaxone and azithromycin on April 26 for initial suspicion for superimposed bacterial pneumonia, however, these were discontinued on discharge.

### Study Assessments

There were no pre-specified study endpoints or mandated procedures. All laboratory tests and radiologic assessments were performed at the discretion of the treating physician and per standard clinical management processes. Vital signs were monitored before and upon completion of each lenzilumab infusion. Demographics, co-existing conditions, laboratory and radiographic data, as well as clinical data, adverse events, and outcomes were captured from the electronic health record until data cutoff on May 1, 2020. Data were for all patients for a minimum of five days following the administration of lenzilumab. Baseline values were defined as those values obtained prior to lenzilumab administration, either on the day of administration or the day before the administration. Cytokine analysis was performed on available serum isolated from patients, pre and post lenzilumab treatment. Serum was diluted 1:2 with assay buffer before following the manufacturer's protocol for Milliplex Human Cytokine/Chemokine MAGNETIC BEAD Premixed 38 Plex Kit (Millipore Sigma, Ontario, Canada). Data were collected using a Luminex (Millipore Sigma, Ontario, Canada).

### Statistical Methods

Continuous variables at baseline are represented using the median and interquartile range (IQR). This is demonstrative of the features in the middle 50% of the cohort. We used an 8-point ordinal outcome scale to define clinical status: 1) Death; 2) Hospitalized, on invasive mechanical ventilation or extracorporeal membrane oxygenation (ECMO); 3) Hospitalized, on non-invasive ventilation or high flow oxygen devices; 4) Hospitalized, requiring supplemental oxygen; 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation on activities; 8) Not hospitalized, no limitations on activities (as recommended by the WHO R&D Blueprint Group). Statistical significance for differences in temperature, serum CRP concentration, serum IL-6 concentration, absolute lymphocyte counts (ALC), and platelet counts on day −1 versus day 3 post-lenzilumab was determined using a two-tailed paired t-test. Day 3 was determined as the last value for statistical analysis as data post day 3 were not available for more than 50% of this cohort.

### Results

### Patients and Baseline Characteristics

Twelve patients received full treatment with 3 doses of lenzilumab administered 8 hours apart. The baseline demographic and clinical characteristics of these patients are summarized in Table 1B. Eight patients (67%) were male; the median age was 65.0 years (range 29-81). Median BMI was 29 (range 22-42). Nine patients were white, 2 were Asian, and 1 American Indian/Native American. All patients had at least one comorbidity associated with poor outcomes. Seven (58%) had diabetes mellitus, 7 (58%) had hypertension, 6 (50%) had obesity (BMI >30), 2 (17%) had chronic kidney disease, 2 (17%) had coronary artery disease and 1 (8%) was on immunosuppressive therapy with a history of kidney transplantation. Seven (58%) had underlying lung disease: 4 (33%) with obstructive sleep apnea, 2 (17%) with chronic obstructive pulmonary disease, and 1 (8%) with reactive airway disease.

All patients required oxygen supplementation at baseline; 1 patient was on non-invasive positive-pressure ventilation, 8 (67%) were on low flow oxygen, 3 (25%) were on high flow oxygen. The median SpO2/FiO2 ratio was 281, with SpO2/FiO2 ratios below 315 in 8 (67%) patients, and below 235 in 3 (25%) patients. Additionally, 6 (50%) patients were febrile within 24-48 hours prior to lenzilumab administration, with a median temperature of 38.3° C.

Seven (58%) patients had lymphopenia at baseline, with an absolute lymphocyte count less than 0.95×10⁹/L. All patients had an elevation in at least one inflammatory marker at baseline. Eleven (92%) patients had elevated CRP values above the upper limit of normal (>8.0 mg/L), with a median of 103.2 mg/L. Ten (83%) patients had elevated ferritin values above the upper limit of normal (>336 mcg/L), with a median of 596 mcg/L. All 11 patients with IL-6 levels available at baseline had elevated values above the upper limit of normal (>1.8 pg/mL), with a median of 30.95 pg/mL. Of the 11 patients with D-dimer levels available at baseline, 9 (75%) had values above the upper limit of normal (>500 ng/mL), with a median of 829 ng/mL.

TABLE 1B
Demographics and baseline characteristics

| Characteristic | Patients (n = 12) | |
|---|---|---|
| Age, years | 65 | (52-70)* |

Gender

| | | |
|---|---|---|
| Male | 8 | (67%) |
| Female | 4 | (33%) |
| BMI | 29 | (24-36)* |

Race

| | | |
|---|---|---|
| White | 9 | (75%) |
| Asian | 2 | (17%) |
| American Indian/Native American | 1 | (8%) |

Comorbidities

| | | |
|---|---|---|
| Diabetes mellites | 7 | (58%) |
| Hypertension | 7 | (58%) |
| Obesity | 6 | (50%) |
| Chronic kidney disease | 2 | (17%) |
| Coronary artery disease | 2 | (17%) |
| Kidney transplantation | 1 | (8%) |
| Obstructive lung disease | 4 | (33%) |
| Chronic obstructive pulmonary disease | 2 | (17%) |
| Reactive airway disease | 1 | (8%) |
| Temperature, ° C. | 38 | (37.25-38.5)* |

Inflammatory markers before lenzilumab
Administration

| | | |
|---|---|---|
| CRP (≤8.0 mg/L) | 103.2 | (52.7-159.9)* |
| Ferritin (Males: 24-336 mcg/L, | 596 | (358.3-709.0)* |
| Females: 11-307 mcg/L) | | |
| IL-6 (≤1.8 pg/mL) | 30.95 | (24.18-34.05)* |
| D-dimer (≤500/ng/mL) | 829 | (513.5-1298.5)* |
| Lymphocyte count before lenzflumab | 0.75 | (0.55-1.04)* |
| administration (0.95-3.07 × 10⁹/L) | | |

Oxygen therapy before lenrilumab
Administration

| | | |
|---|---|---|
| High-flow oxygen | 3 | (25%) |
| Nasal cannula | 8 | (67%) |
| Invasive ventilation | 0 | (0%) |
| Noninvasive ventilation | 1 | (8%) |
| SpO2/FiO2 ratio before lenzflumab | 280.9 | (252.5-317.9)* |
| administration | | |

*Median (IQR)

### Clinical Outcomes

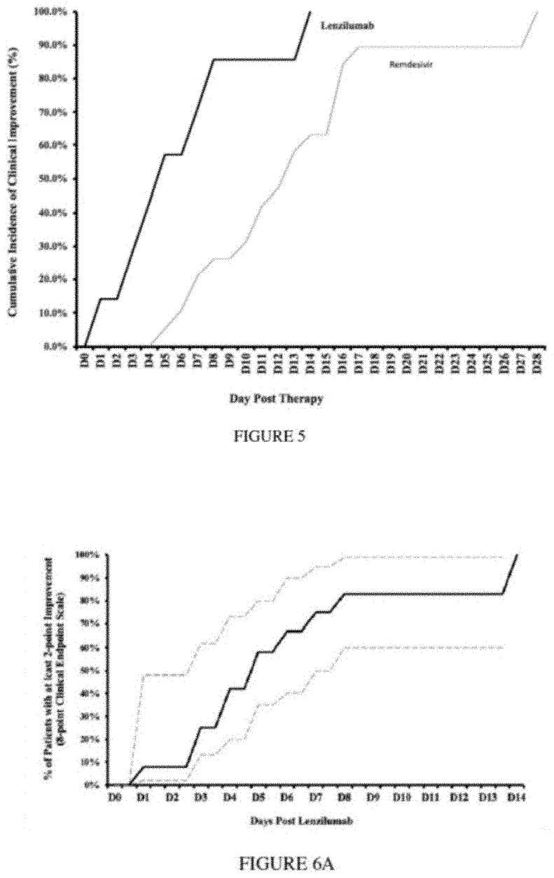

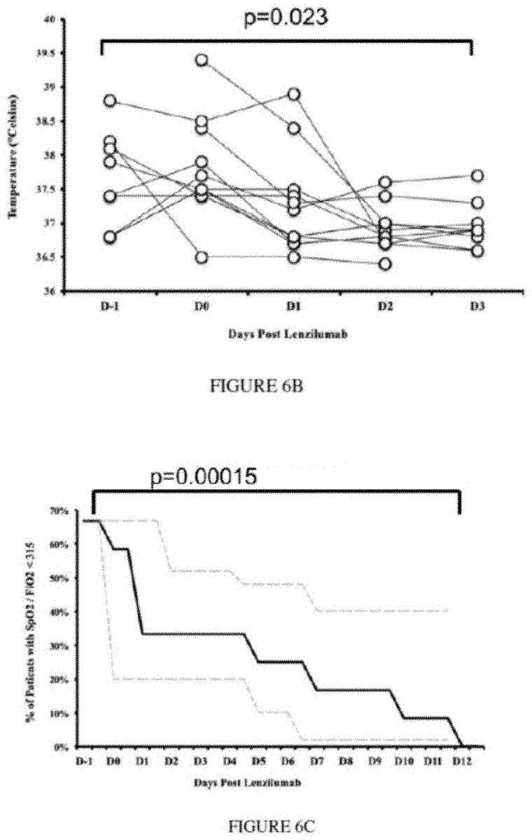

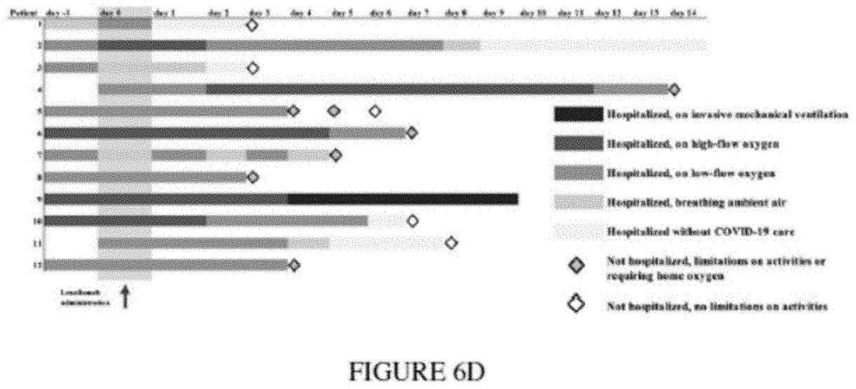

Clinical improvement, as defined by the improvement of at least 2 points on the 8-point ordinal clinical endpoints scale, was observed in 11 out of 12 (92%) patients (FIG. 6A), with a ≥3-point improvement in 10 patients and a 2-point improvement in 1 patient (FIG. 6A). (Table 5). The median time to a 2-point clinical improvement was 5 days (95% CI, 2-7 days). All 11 patients with clinical improvement were discharged after a median of 5 days (range 3-19) post-lenzilumab. The patient discharged on day 19 was ready for discharge on day 9 but remained hospitalized for social reasons. As shown in Table 6, the time to clinical two point improvement was more than 50% faster after treatment with Lenzilumab, which had mean days to discharge of 6.3 days compared mean days to discharge of 13.7 days after treatment with Remdesivir, mean days to discharge of 13 days after treatment with Lopinavir-Ritonavir, and mean days to discharge of 13.5 days after treatment with Tocilizumab. Table 7 shows comparative published data from a remdesivir CU cohort, which indicates a slower mean time to improvement and discharge (adapted from Grein et al., NEJM, Apr. 10, 2020, which is incorporated by reference herein in its entirety.)

There was a significant improvement in mean temperature at day 3 compared to baseline (37.95 vs. 36.97, p=0.023, FIG. 6B and FIG. 10 (up to day 6, p=0.0029). In patients who were febrile at baseline, fever resolved within 48 hours of lenzilumab administration. There was a significant improvement in the proportion of patients with SpO2/FiO2<315 at the end of observation compared to baseline (8% vs. 67%, p=0.00015 SpO2/FiO2 level baseline vs. last value, FIG. 6C). Of 8 patients with SpO2/FiO2<315 at baseline, SpO2/FiO2 improved to >315 in four on day 1 post-lenzilumab. Five (42%) patients were discharged on home oxygen, including one patient who had been on home oxygen pre-COVID-19 illness. One patient (8.3%) required invasive mechanical ventilation. There were no deaths. FIG. 6D depicts individual patient hospitalization and oxygen requirement status.

### Laboratory Markers

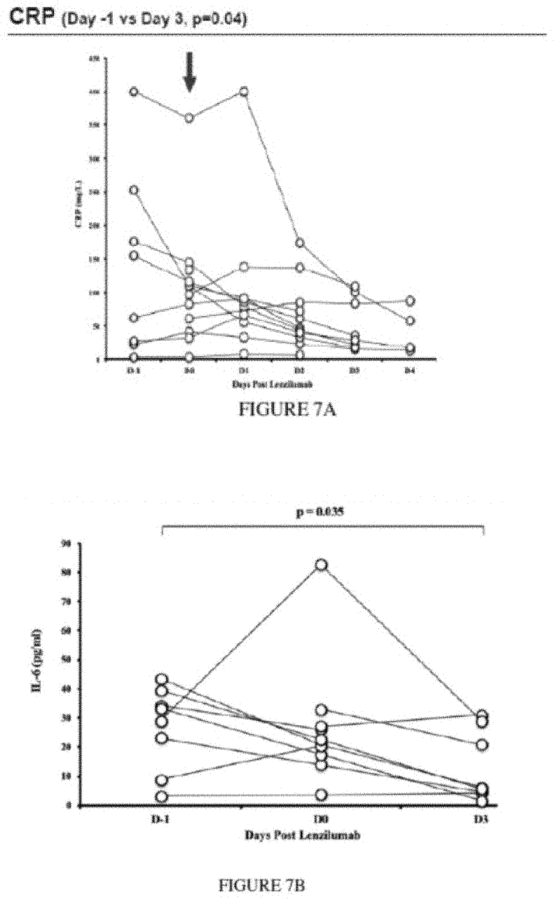

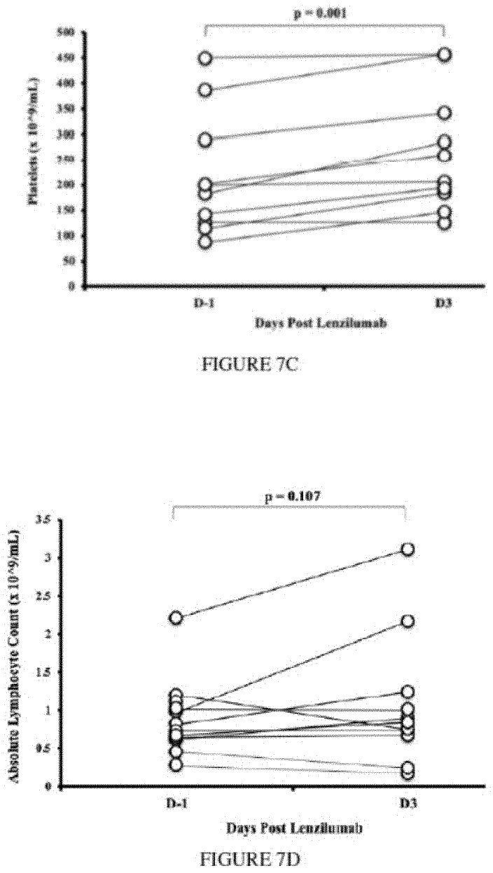

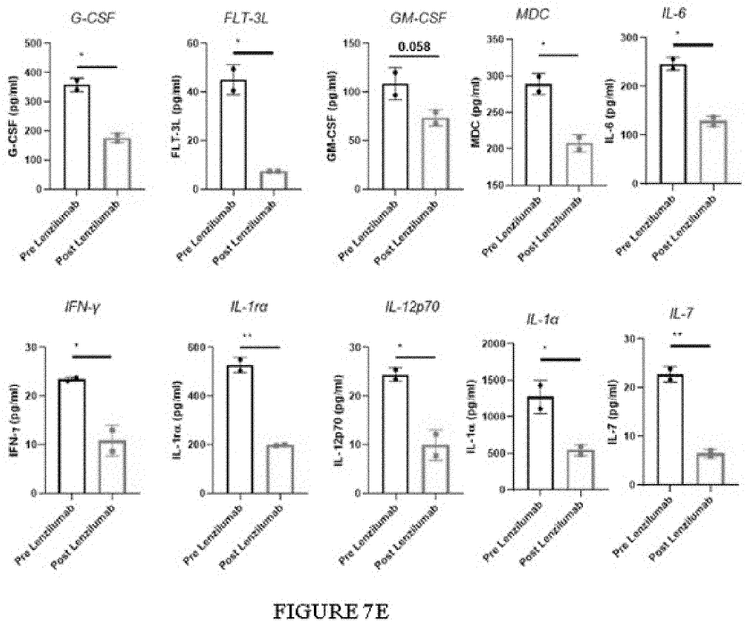

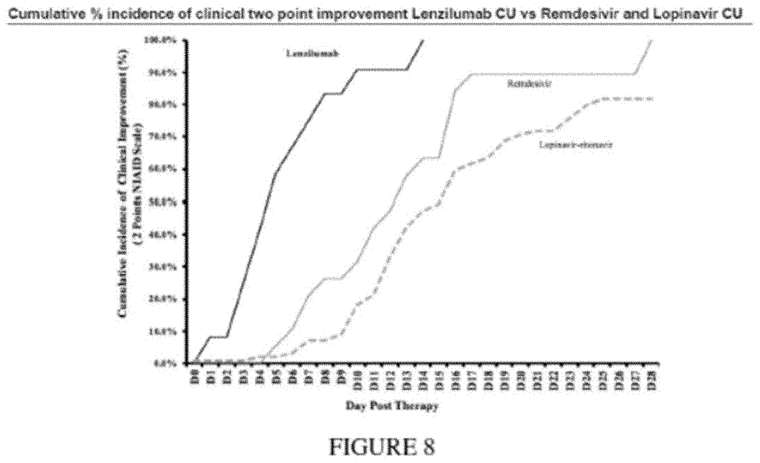

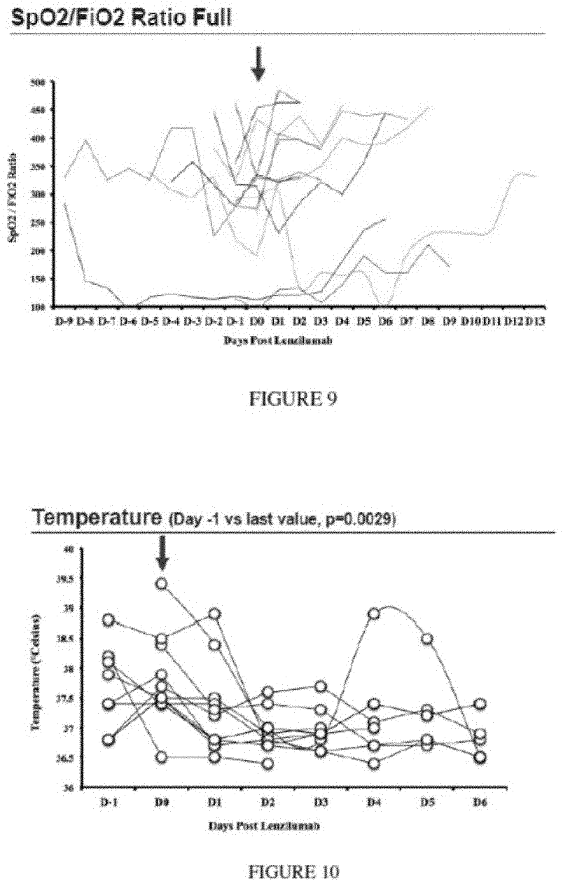

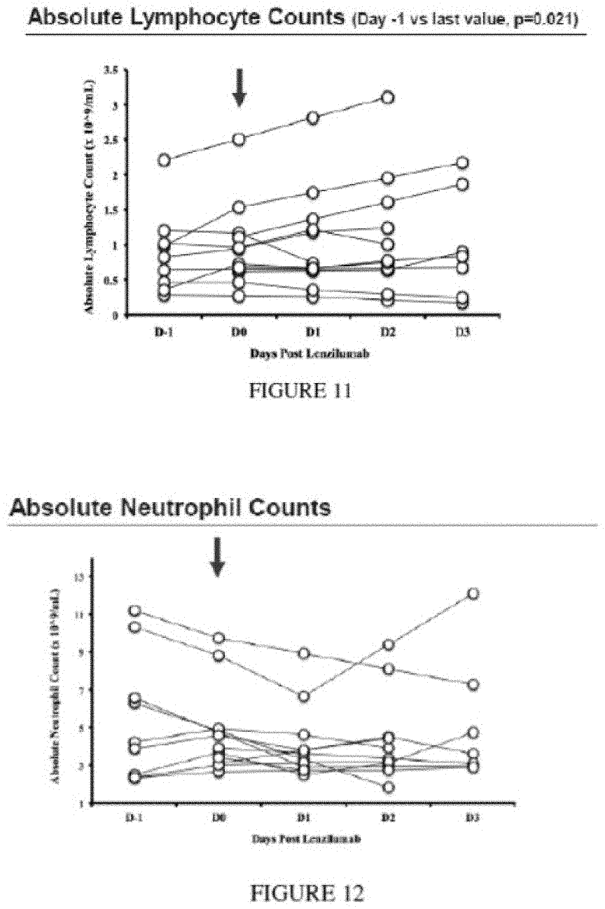

Compared to baseline, there was significant improvement in mean CRP and IL-6 on day 3 following lenzilumab administration (137.3 mg/L vs. 51.2 mg/L, p=0.040; 26.8 pg/mL vs. 16.1 pg/mL, p=0.035; respectively) (FIGS. 7A, 7B). Compared to baseline, an improvement of at least 50% was observed in CRP levels in 6 patients (50%) by day 2, and IL-6 levels in 4 patients (33.3%) by day 3. There was a significant increase in mean platelet count from baseline to day 3 post lenzilumab (217.7×10⁹/L vs 261.8×10⁹/L, p=0.001, FIG. 7C). There was also a trend toward improved absolute lymphocyte counts on day 3 compared to baseline (0.89×10⁹/L vs 1.14×10⁹/L, p=0.107, FIG. 7D and FIG. 11 p=0.021). Analysis of human cytokines comparing pretreatment with 48 hours post-lenzilumab treatment in one patient revealed significant reduction in multiple cytokines involved in the cytokine storm (G-CSF, MDC, GM-CSF, IL-1α, IFN-γ, IL-7, FLT-3L, IL-1rα, IL-6, IL-12p70, FIG. 7E).

### Safety of Lenzilumab Treatment

There was no significant difference in mean absolute neutrophil count or hemoglobin values between baseline and day 3 post lenzilumab: 5.1×10⁹/L vs. 4.8×10⁹/L, p=0.27; 12.9 g/dL vs. 11.4 g/dL, p=0.89; respectively. In one patient, hemoglobin values dropped from 10.3 g/dL on day 0 to 7.9 g/dL on day 6. This patient had undergone a renal biopsy on day 2; imaging revealed a subcapsular hematoma. At the last study observation, the patient remained anemic at 9.3 g/dL.

There were no infusion reactions with lenzilumab administration. One patient, with a history of restless leg syndrome, reported a “pins and needles” sensation during the first dose of lenzilumab; those symptoms resolved and did not recur with subsequent infusions of lenzilumab. No other treatment-emergent adverse events attributable to lenzilumab were noted.

TABLE 2
Summary CBC Labs for Patients Treated
with Lenzilumab on a Compassionate Use

| Neutrophils | | | |
|---|---|---|---|
| Patient | 1st Lab | High | Last |
| 1 | 6.31 | 6.31 | 1.85 |
| 2 | 3.77 | 4.92 | 4.92 |
| 3 | 2.34 | 2.74 | 2.74 |
| 4 | 3.1 | 4.85 | 3.11 |
| 5 | 3.94 | 11.22 | 7.28 |
| | 3.892 | 6.008 | 3.98 |

| Lymphocytes | | | |
|---|---|---|---|
| Patient | 1st Lab | Low | Last |
| 1 | 2.21 | 2.21 | 3.11 |
| 2 | 0.27 | 0.17 | 0.17 |
| 3 | 1.2 | 0.74 | 0.74 |
| 4 | 0.61 | 0.57 | 0.92 |
| 5 | 0.93 | 0.87 | 2.17 |
| | 1.044 | 0.912 | 1.422 |

| | Last Value | High/Low * |
|---|---|---|
| Neutrophils | 3.98 | 6.008 |
| Lymphocytes | 1.422 | 0.912 |

* High is the peak level of neutrophils and Low is the lowest value of lymphocytes

The trends in a reduction of neutrophils (from peak value) and an increase of lymphocytes (from low value) are consistent with the mechanism of action (MOA) of lenzilumab and are an indication of its therapeutic effect together with a decrease of inflammatory markers, including but not limited to CRP, serum ferritin, D-dimer and interleukin 6 (IL-6). These results align with the demonstration by Wu C et al. JAMA Intern Med. Published online Mar. 13, 2020. doi:10.1001/jainainternmed.2020.0994, which is incorporated herein by reference in its entirety, as shown in Table 3.

TABLE 3
Hematologic Data of Patients with or without ARDS
(adapted from Wu et al. JAMA Mar. 13, 2020)

| | No ARDS | ARDS |
|---|---|---|
| Hematologpc Levels | Mean and Range | Mean and Range |
| White Blood Cells, ×10⁹ | 5.02 (3.37 to 7.18) | 8.32 (5.07 to 11.20) |
| Neutrophils, ×10⁹ | 3.06 (2.03 to 5.56) | 7.04 (3.98 to 10.12) |
| Lymphocytes, ×10⁹ | 1.08 (0.72 to 1.45) | 0.67 (0.49 to 0.99) |

TABLE 4
Patient Lab Data to Date (Apr. 13, 2020) for Complete Blood Count and Clinical Markers
of CRS, Including Inflammatory Markers and ARDS Risk Factor (Ferritin Level of >300
mcg/L), in Patients Before and Post Lenzilumab Treatment on a Compassionate Use

| | | | B/L | | | | | | | | | | |
|---|---|---|---|---|---|---|---|---|---|---|---|---|---|
| | | | Lymph | B/L | | | | | | | | IL-6 | CRP |
| | B/L | B/L | count × | PLT | B/L | B/L | | B/L | B/L | Peak | B/L | post | post |
| | HgB, | WBC × | 10 9/L | count × | AST | ALT | CRP | LDH | Ferritin | Ferritin | IL-6 | Rx | Rx |
| Pt | g/dL | 10 9/L | or % | 10 9/L | U/L | U/L | mg/L | U/L | mcg/L | mcg/L | Pg/mL | Pg/mL | mg/L |
| 1 | 15 | 9.5 | 2.21 | 182 | 31 | 22 | 100 | | 273 | 299 | 2.2 | | 46 |
| | | | | | | | | | (4/7) | (4/9) | (4/8) | | |
| 2 | 8.7 | 4.4 | 0.27 | 241 | 23 | 6 | 29.7 | 283 | 548 | 1143 | 34.7 | | 22.4 |
| | | | | | | | (4/3) | (4/6) | (4/3) | 4/9 | (4/3) | | |
| | | | | | | | 22.2 | 235 | | | 26.2 | | |
| | | | | | | | (4/5) | (4/4) | | | (4/6) | | |
| | | | | | | | 41.2 | | | | | | |
| | | | | | | | (4/6) | | | | | | |
| 3 | 16.7 | 4.1 | 1.2 | 126 | 21/2 | | 7.2 | | 356 | 571 | 3.1 | | ‘Low’ |
| | | | | | 6/24 | | (4/7) | | (4/5) | (4/8) | (4/5) | | |
| | | | | | | | | | | | 4.3 | | |
| | | | | | | | | | | | (4/7) | | |
| 4 | 15.9 | 4.5 | 0.61 | 126 | 43 | 35 | 44.9 | 282 | 519 | | 27.1 | | |
| | | | | | 49 | 70 | 61.2 | 388 | | | 95.4 | | |
| | | | | | | | 175.8 | | | | | | |
| 5 | 14.2 | 6.3 | 0.87 | 163 | 72 | 41 | 53.4 | — | 1269 | 1269 | 23.2 | 4.6 | 22.0 |
| | | | | | 101 | 98 | | | | | (3/3) | (4/5) | |
| | | | | | 183 | 190 | 184.4 | | | | | | |
| | | | | | 96 | 175 | | | | | | | |

Pt = Patient; B/L = Blood level; HgB = Hemoglobin; WBC = White blood cells; Lymph = Lymphocytes; PLT = Platelet; AST = aspartate aminotransferase; ALT = alanine aminotransferase; CRP = C reactive protein; LDH = lactate dehydrogenase; ferritin is serum ferritin; and interleukin 6 = IL-6.

TABLE 5
Lenzilumab CU Data Rapid Clinical Improvement and Discharge

| | | | | | | | | Days to 2 | |
|---|---|---|---|---|---|---|---|---|---|
| | | | | | | | | point | |
| | | | | | | | | clinical | Days to |
| | | | | | | | Other; | improvement | discharge |
| | | | | | | | therapies | from | from |
| | | | | | | Oxygen | tried and | first dose of | first |
| Patient# | Age | Sex | Race | BMI | Comorbidities | Requirement | stopped | Lenzilumab | dose |
| 1 | 29 | F | Caucasian | 30 | Obese | Low- | Transferred | 1 | 3 |
| | | | | | | flow | to ICU prior | | |
| | | | | | | Oxygen | to | | |
| | | | | | | | lenzilumab; | | |
| | | | | | | | azithromycin, | | |
| | | | | | | | ceftriaxone | | |
| 2 | 62 | F | Asian | 23 | Kidney transplant, | High- | Transferred | 8 | 9 |
| | | | | | HTN, DM, CHF, | flow | to ICU prior | | |
| | | | | | CKD, | Oxygen | to | | |
| | | | | | immunocompromised | | lenzilumab; | | |
| | | | | | | | cefepime, | | |
| 3 | 38 | M | Asian | 22 | Ex-smoker, latent TB | Low- | HCQ | 3 | 3 |
| | | | | | | flow | | | |
| | | | | | | Oxygen | | | |
| 4 | 68 | M | Caucasian | 28 | CAD, HTN, | Low- | HCQ, | 14 | 14 |
| | | | | | overweight | flow | tocilizumab, | | |
| | | | | | | Oxygen | cefepime, | | |
| | | | | | | | azithromycin | | |
| 5 | 55 | M | Caucasian | 36 | Reactive airway | Low- | Remdesivir, | 4 | 4 |
| | | | | | disease, very obese | flow | azithromycin, | | |
| | | | | | | Oxygen | ceftriaxone | | |
| 6 | 75 | M | Caucasian | 26 | HTN, DM, COPD, | High- | HCQ, | 7 | 7 |
| | | | | | CKD, overweight | flow | ceftriaxone, | | |
| | | | | | | Oxygen | doxycycline | | |
| 7 | 69 | M | Unknown | 36 | HTN, DM, very | Low- | Ceftriaxone | 5 | 5 |
| | | | | | obese | flow | | | |
| | | | | | | Oxygen | | | |
| 8 | 41 | M | Caucasian | 35 | Ex-smoker, Vaping, | Low- | — | 3 | 3 |
| | | | | | BMI ≥28 | flow | | | |
| | | | | | | Oxygen | | | |
| 9 | 81 | M | Unknown | 24 | HTN, CKD, pre-DM | High- | Transferred | — | — |
| | | | | | | flow | to ICU prior to | | |
| | | | | | | Oxygen | lenzilumab; | | |
| | | | | | | | azithromycin, | | |
| | | | | | | | steroids | | |
| 10 | 59 | F | Caucasian | 37 | HTN, DM, BMI ≥28 | NIPPV | Transferred | 6 | 7 |
| | | | | | | | to ICU prior | | |
| | | | | | | | to lenzilumab | | |
| 11 | 73 | M | Caucasian | 23 | DM | Low- | Nursing | 5 | 8 |
| | | | | | | flow | home | | |
| | | | | | | Oxygen | resident | | |
| 12 | 68 | F | Caucasian | 42 | CAD, HTN, DM, | Low- | — | 4 | 4 |
| | | | | | BMI ≥28, COPD, | flow | | | |
| | | | | | OSA, Ex-smoker | Oxygen | | | |

HTN = hypertension; DM = diabetes mellitus; CHF = congestive heart failure; CKD = chronic kidney disease; TB = tuberculosis; CAD = coronary artery disease; HCQ = hydroxychloroquine.
All 12 patients survived.
4 patients were in ICU prior to receiving lenzilumab.
11 of 12 patients were discharged, the median time to discharge was 5 days.
All patients had at least one co-morbidity associated with worsened outcome and at least two inflammatory elevated biomarkers indicative of high risk of progression (CRP, Ferritin, D-dimer, and/or LDH)

TABLE 6
Lenzilumab Compassionate Use Patients vs Remdesivir -
Patient Baseline Characteristics

| | Remdesivir | Lopinavir/Ritonavir | Tocilizumab | Lenzilumab¹ |
|---|---|---|---|---|
| Median age | 53.0 | 58.0 | 56.8* | 65.0 |
| Male | 68% | 60% | 86% | 67% |
| <50 | 42% | Nd | Nd | 25% |
| 50 to <70 | 37% | Nd | nd | 50% |
| >70 | 21% | Nd | nd | 25% |
| Comorbidities | | | | |
| Hypertension | 21% | nd | 43% | 58% |
| Diabetes | 5% | 12% | 24% | 58% |
| Asthma | 5% | nd | nd | 8% |
| BMI >=28 | nd | nd | nd | 58% |
| Oxygenation Status | | | | |
| IMV | — | — | 10% | — |
| NIPPV | 11% | — | 10% | 8% |
| High-Flow | 26% | 16% | 45% | 25% |
| Low-Flow | 53% | 70% | 35% | 67% |
| Ambient Air | 11% | 14% | 0% | 0% |

| | | Lopinavir/ | | |
|---|---|---|---|---|
| *mean | Remdesivir | Ritonavir | Tocilizumab | Lenzilumab |
| Mean Days to | 13.7 | 13** | 13.5 | 63 |
| Discharge | | | | |
| Range | (6-28)* | Nd | (10-19)* | (3-14)*** |

*2 patients not discharged
**median, 35.7% of patients discharged on day 14
***patient 9 pending
¹Lenzilumab patients were older, had more comorbidities, similar oxygenation status, but discharged much earlier.

TABLE 7
Remdesivir Cohort: Slower to Improvement and Discharge -
Compassionate Use Data (adapted from Grein et al., NEJM, Apr. 10,
2020, which is incorporated by reference herein in its entirety.)

| | | | Days to | |
|---|---|---|---|---|
| | | | Improvement | Days to |
| | Patient | | from First | Discharge from |
| Baseline | No. | Outcome | Dose*,^{A} | First Dose^{B} |
| NIPPY | 35 | Discharged | 4 | 10 |
| NIPPV | 36 | Discharged | 2 | 17 |
| High-flow oxygen | 37 | Death | — | — |
| High-flow oxygen | 38 | Hospitalized | — | — |
| High-flow oxygen | 39 | Discharged | 7 | 28 |
| High-flow oxygen | 40 | Discharged | 6 | 23 |
| High-flow oxygen | 41 | Discharged | 3 | 26 |
| Low-flow oxygen | 42 | Discharged | 13 | 14 |
| Low-flow oxygen | 43 | Discharged | 13 | 13 |
| Low-flow oxygen | 44 | Discharged | 9 | 13 |
| Low-flow oxygen | 45 | Discharged | 10 | 11 |
| Low-flow oxygen | 46 | Discharged | 8 | 8 |
| Low-flow oxygen | 47 | Discharged | 7 | 16 |
| Low-flow oxygen | 48 | Discharged | 4 | 7 |
| Low-flow oxygen | 49 | Discharged | 1 | 16 |
| Low-flow oxygen | 50 | Discharged | 1 | 12 |
| Low-flow oxygen | 51 | Discharged | 1 | 6 |
| Ambient air | 52 | Discharged | 16 | 16 |
| Ambient air | 53 | Discharged | 11 | 11 |

| | | |
|---|---|---|
| Average | 6.82 days | 13.69 days |

*If patient improved and subsequently worsened, time of improvement from worsened condition Used. If patient did not improve, discharge date used as date of improvement.
^{A}Average time to improvement from first dose = 6.82 days.
^{B}Average time to discharge from first dose = 13.69 days.

### Discussion

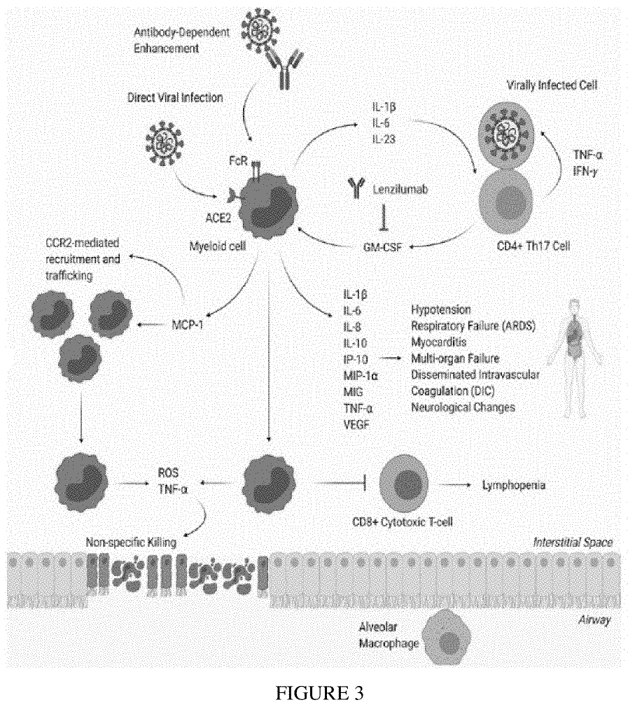

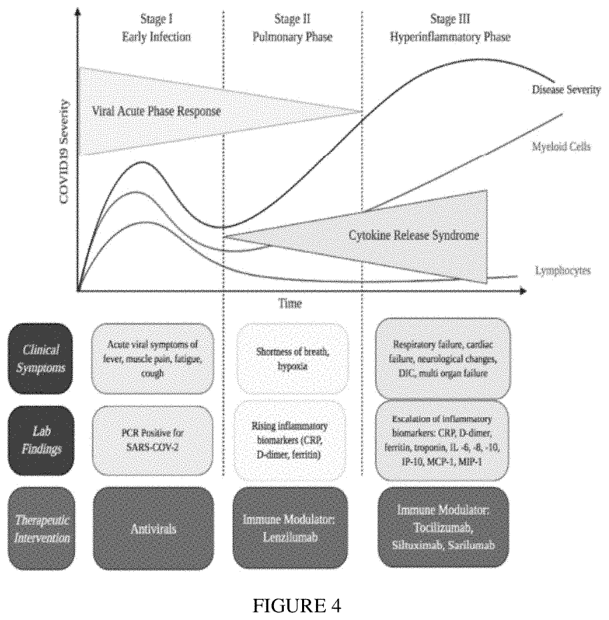

There is no therapy with proven efficacy against COVID-19 at present. Observations from the first-ever use of lenzilumab to neutralize GM-CSF in the treatment of COVID-19 are reported here. Lenzilumab was offered through a compassionate single-use IND to patients with severe and critical COVID-19 pneumonia. Based on the pathophysiology of cytokine storm following SARS-CoV-2 infection, along with our preclinical work, it was hypothesized that lenzilumab-induced GM-CSF depletion prevents CRS in COVID-19 and progression to severe disease or death. At baseline, all 12 patients had at least one risk factor associated with poor outcomes: age, smoking history, cardiovascular disease, diabetes, chronic kidney disease, chronic lung disease, high BMI, and elevated inflammatory markers, with several patients having multiple such risk factors. In this cohort of high-risk patients with severe and critical COVID-19 pneumonia, treatment with lenzilumab was associated with improved overall clinical outcome in 11/12 patients (91.7%) on an 8-point ordinal scale; all 11 patients were discharged after a median of 5 days. Significant improvement in oxygen requirement, as well as inflammatory cytokines and markers of disease severity, were also observed. These results are consistent with our original hypothesis, and corroborate our laboratory findings following GM-CSF depletion in preclinical models of CRS after CART cell therapy. In addition, the use of lenzilumab was associated with a significant improvement in platelet count, indicating possibly an overall improved coagulopathy associated with CRS post-COVID-19. Interestingly, the use of lenzilumab in this cohort was associated with a trend to an increase in lymphocyte count (FIG. 7D). It was recently shown that GM-CSF depletion results in modulation of apoptosis pathways in T cells. It is unclear at this time if the increase in lymphocyte count is secondary to clearance of SARS-CoV-2 virus, or a direct effect of GM-CSF on T cells; this question will be answered in the planned phase III trial. FIG. 3 depicts a proposed mechanism for the role of GM-CSF in CRS post-COVID-19.

Five patients received other pharmacotherapies targeting COVID-19 besides lenzilumab. Three patients received hydroxychloroquine; one patient received remdesivir and one patient received steroids. Two patients received lenzilumab after the failure of clinical improvement with either hydroxychloroquine or remdesivir and subsequently improved. Two patients received lenzilumab concomitantly with hydroxychloroquine; both of these patients were discharged home. One of these patients also received off-label tocilizumab on day 6 post-lenzilumab and was released on home oxygen. One patient received steroid therapy concomitantly with lenzilumab; this patient remained on invasive mechanical ventilation on the last day of observation.

The use of lenzilumab was safe, without any adverse events attributable to lenzilumab. While there is a theoretical concern for bone marrow toxicity when GM-CSF is depleted, lenzilumab treatment was not associated with any hematological toxicity in this cohort. There were no infusion reactions following lenzilumab treatment. Importantly, a sensation of pins and needles reported by one patient while receiving lenzilumab, did not recur with subsequent infusions; the patient had a history of restless leg syndrome. Restless legs have not been described in any of the non-COVID-19 patients who have received lenzilumab for other indications.

The present report has several limitations. First, the sample size is small and did not include controls. Second, as lenzilumab was offered under emergency single-use IND conditions, all management decisions, including prescribing medications and laboratory/radiologic monitoring, were at the discretion of the treating clinicians. This resulted in some heterogeneity in the treatment specifics of individual patients as well as the laboratory and other diagnostic data that were collected. Given this and the absence of a control arm in the study, it cannot, with full confidence, be declared that the clinical improvement that was noted in our patients was clearly and solely attributable to lenzilumab. These limitations will be addressed in the recently initiated randomized Phase III clinical trial (NCT04314843).

In summary, lenzilumab was administered, under a single-use emergency IND compassionate program, to 12 patients with severe and critical COVID-19 pneumonia and with risk factors for disease progression. Lenzilumab use was associated with improved clinical outcomes, oxygen requirement, and cytokine storm in this cohort of patients, with no reported mortality. Lenzilumab was well tolerated; no treatment-emergent adverse events attributable to lenzilumab were observed.

### Example 9

### A Phase 3 Randomized, Placebo-Controlled Study of Lenzilumab in Hospitalized Patients with Severe and Critical COVID-19 Pneumonia

Most deaths in COVID-19 patients result from respiratory distress, which appears to be driven in large part by a CRS mediated hyper-immune reaction (‘cytokine storm’) that may occur even in patients who appear to be resolving their infection by viral titers. In addition, GM-CSF+ T cells are highly correlated with severity and ICU admission in the setting of COVID-19. For this reason, it is critical to intervene prior to the initiation of CRS and severe respiratory distress in patients at high risk of progression.

The primary objective of this study is to assess whether the use of lenzilumab in addition to current standard of care (SOC) can alleviate the immune-mediated cytokine release syndrome (CRS) and reduce time to recovery in patients with severe or critical COVID-19 pneumonia.

A secondary study objective is to assess the safety profile and incidence of invasive mechanical ventilation (IMV) and/or death, clinical improvement using the clinical endpoint 8-point ordinal scale, incidence of severe ARDS, difference, change in mean hemophagocytic lymphohistiocytosis (HLH) score and health resource utilization (including impact on duration of hospitalization, intensive care unit (ICU) admission, use of high flow or low flow oxygen therapy and/or vasopressor support) of lenzilumab vs. placebo alongside current standard of care in hospitalized subjects with severe or critical COVID-19 pneumonia.

A secondary study objective is to assess the safety profile and incidence of invasive mechanical ventilation (IMV) and/or death, clinical improvement using the clinical endpoint 8-point ordinal scale, incidence of severe ARDS, difference, change in mean hemophagocytic lymphohistiocytosis (HLH) score and health resource utilization (including impact on duration of hospitalization, intensive care unit (ICU) admission, use of high flow or low flow oxygen therapy and/or vasopressor support) of lenzilumab vs. placebo alongside current standard of care in hospitalized subjects with severe or critical COVID-19 pneumonia.

The study hypothesis is that the use of lenzilumab in addition to current standard of care will alleviate the immune-mediated CRS and reduce the time to recovery in this patient group by 33%.

The primary endpoint is the time to recovery by Day 28 based on the 8-point clinical status ordinal scale.

Secondary endpoints are:

Change from baseline to Day 28 in clinical status based on the 8-point ordinal scale,
Time to improvement in 1 category using 8-point ordinal scale up to Day 28,
Time to improvement in 2 categories using 8-point ordinal scale up to Day 28,
Incidence of use of IMV and/or death up to Day 28,
Incidence of severe ARDS up to Day 28,
Difference in mean HLH score up to Day 28,
Duration of hospitalization up to Day 60,
Time to hospital discharge up to Day 60,

### Incidence of IMV (or use of Extracorporeal Membrane Oxygenation) up to Day 28,

Ventilator-free days up to Day 28,
Organ failure-free days up to Day 28,
Incidence of ICU stay up to Day 28,
Duration of ICU stay up to Day 28,
Incidence of low-flow supplemental oxygen use up to Day 28,
Duration of time on supplemental oxygen (low-flow or high-flow) up to Day 28,
Time to improvement in oxygenation for >48 hours up to Day 28,
Increase in SpO2/FiO2 of 50 or greater compared to the nadir SpO2/FiO2 up to Day 28,
Time to clinical improvement, which is defined as National Early Warning Score 2 (NEWS2) of <2 maintained for 24 hours up to Day 28, NEWS2 consists of: Physiological Parameters: respiration rate (per minute), SpO2 Scale 1(%), SpO2 Scale 2(%), use of air or oxygen, systolic blood pressure (mmHg), pulse (per minute), consciousness and temperature (° C.),
Incidence of non-invasive ventilation (or use of high-flow oxygen device) up to Day 28,
Number of subjects alive and off of oxygen up to Day 60,
Incidence of adverse events (AE) based on the National Cancer Institute (NCI) Common Terminology Criteria for Adverse Events (CTCAE) version 5.0 up to Day 28,
Incidence of serious adverse events (SAE) based on the NCI CTCAE version 5.0 up to Day 60,
and Proportion of subjects alive at Day 60.

### Study Design

This is a phase 3, prospective, randomized, multicenter, double-blind, placebo-controlled clinical trial evaluating the use of lenzilumab or placebo alongside current standard of care for the reduction in time to recovery at Day 28 (using the 8-point clinical endpoint ordinal scale) in hospitalized subjects with severe or critical COVID-19 pneumonia. A total of approximately 300 subjects will be enrolled in one of two treatment groups. Subjects will be randomized to receive lenzilumab or placebo in a 1:1 ratio (lenzilumab (n=150) or placebo (n=150)) alongside standard of care. Subjects will be stratified upon randomization by age (<65 years vs. ≥65 years) and disease severity (severe vs. critical). A prespecified interim analysis will be conducted by the Data Safety and Monitoring Board (DSMB) when 50% of the expected events (recoveries) have occurred to perform an unblinded futility assessment and a sample size reassessment. Subjects will be followed out to Day 60.

The current protocol for the Phase III study will have the following inclusion and exclusion criteria:

Inclusion Criteria:

1. Adults 18 to 85 years of age, inclusive, who are capable of providing informed consent or have a proxy capable of giving consent for them.
2. Virologic confirmation of SARS-CoV-2 infection via any FDA authorized diagnostic test for SARS-CoV-2 (e.g. qualitative SARS-CoV-2 real time polymerase chain reaction (RTPCR), nucleic acid amplification (molecular) test, etc.) assessed locally per institution standard of care, prior to randomization.
3. COVID-19 pneumonia diagnosed by chest x-ray or computed tomography (CT) revealing infiltrates consistent with pneumonia. Note that a CT scan may be used if available, but is not required.
4. Subject must have an SpO2≤94% on room air and/or require supplemental oxygen to be eligible.
5. Subject is hospitalized and has not required invasive mechanical ventilation during this hospitalization.
6. Subject has not participated in other clinical trials for COVID-19. Note that subjects on corticosteroids, remdesivir or other anti-virals and/or hydroxychloroquine with or without azithromycin are not excluded from the study. Participation in remdesivir clinical trials is permitted provided that the subject meets all other eligibility criteria. Agents that have received emergency use authorization from the FDA are permitted provided they are not immunomodulators and subjects who have received convalescent plasma are not excluded.
7. Females of childbearing potential must have a negative serum pregnancy test at screening/baseline. Women of childbearing potential must agree to use adequate contraception (hormonal or barrier method of birth control, abstinence) prior to study entry and for 5 months following their last dose of study drug. A negative serum beta human chorionic gonadotropin ((3-hCG) is required for all women of childbearing potential within 1 week prior to receiving first dose of study drug.

Exclusion Criteria:

1. Subject requires invasive mechanical ventilation or extracorporeal membrane oxygenation (i.e., category 2 on the ordinal scale).
2. Confirmed diagnosis of bacterial pneumonia or other active/uncontrolled fungal or other viral infections at screening/baseline.
3. Known active tuberculosis (TB), history of incompletely treated TB or suspected or known extrapulmonary TB.
4. Currently receiving treatment for hepatitis A, hepatitis B, hepatitis C or HIV infection.
5. History of pulmonary alveolar proteinosis (PAP).
6. Women of childbearing potential who are pregnant or breastfeeding.
7. Known hypersensitivity to lenzilumab or any of its components.
8. Use of anti-IL-6 therapy or any other immunomodulatory or immunosuppressive therapy or live vaccine within 8 weeks prior to randomization.
Note: Subjects on corticosteroids are not excluded from the study.
Note: Subjects on remdesivir or other anti-virals and/or hydroxychloroquine with or without azithromycin or who have received convalescent plasma are not excluded from the study.
9. Use of GM-CSF agents (e.g., sargramostim) within 2 months prior to randomization.
10. Expected survival <24 h in the opinion of the investigator.
11. Any condition that, in the opinion of the investigator, is likely to interfere with the safety and efficacy of the study treatment or puts the subject at unacceptably high risk from the study.

### Excluded Medications

The following medications are prohibited prior to randomization into the study:
Anti-IL-6 therapy or any other immunomodulatory or immunosuppressive therapy or live vaccine within 8 weeks prior to randomization.
GM-CSF agents (e.g., sargramostim) within 2 months prior to randomization.
During the study (i.e., prior to Day 28) the following medications are prohibited:
GM-CSF agents (e.g., sargramostim).
Anti-IL-6 therapy or any other immunomodulatory or immunosuppressive therapy or live vaccine (note: use of corticosteroids is allowed).
Other investigational therapies to treat COVID-19 related symptoms.

### Definitions

Severe is defined as: SpO2≤94% on room air or requiring low-flow oxygen support

Critical is defined as meeting at least one of the following criteria:
Requiring high-flow oxygen support or non-invasive positive pressure ventilation (NIPPV),
Shock (defined by systolic blood pressure (bp)<90 mmHg or diastolic <60 mmHg or requiring vasopressors), or
Multi-organ dysfunction/failure.

Treatment will be administration of lenzilumab 600 mg intravenously (IV) beginning on Day 0 within 12 hours of randomization. Three (3) doses of lenzilumab will be administered with 8 hours (±30 minutes) between each dose (i.e., 1,800 mg over 24 hours). Lenzilumab will be administered in a total volume of 250 mL over 60 minutes.

The following medications should be administered approximately 1 hour prior to each lenzilumab infusion to prevent infusion reactions.
Acetaminophen 500 to 1000 mg PO or IV
Diphenhydramine 12.5 to 25 mg IV, or 25 mg PO or equivalent.

Alternatives to the recommendations should be discussed with the medical monitor.

Placebo is commercially sourced preservative-free 0.9% sodium chloride solution for injection that will be administered in a manner identical to lenzilumab.

Subjects will continue to receive institutional standard of care for the treatment of COVID-19 pneumonia and other conditions. The use of glucocorticosteroids, hydroxychloroquine,

azithromycin, remdesivir or other anti-viral therapy is permitted.

### Example 10

Given the hypothesized role of GM-CSF in the pathogenesis of COVID-19 related immune hyper-response, along with prior studies demonstrating that GM-CSF depletion prevents CRS and modulates myeloid cell behavior in preclinical models, lenzilumab therapy was offered to patients hospitalized with severe COVID-19 pneumonia, who had clinical and/or biomarker evidence for increased risk of progression to respiratory failure.

### Methods

### Patients

Hospitalized patients with COVID-19, confirmed by reverse transcriptase-polymerase chain reaction for the SARS-CoV-2, and radiographic findings consistent with COVID-19 pneumonia were considered for treatment with lenzilumab through an emergency investigational new drug (IND) program. Active systemic infection with bacteria, fungi, or other viruses, was an exclusion criterion. Informed consent and Institutional Review Board approval was obtained for each patient. A request for lenzilumab under FDA emergency use IND was submitted to the FDA in accordance with agency guidelines (fda.gov/regulatory-information/search-fda-guidance-documents/emergency-use-investigational-drug-or-biologic). All subjects received lenzilumab 600 mg administered via a 1-hour intravenous infusion every 8 hours for a total of three doses (1800 mg). A control cohort was identified from an electronic registry of more than 1900 COVID-19 patients in the same healthcare centers as cases, who did not receive lenzilumab, but matched cases on sex and age within a tolerance of 5 years. Patients in the untreated group were further matched to patients in the lenzilumab group for disease severity (hospitalized with COVID-19 pneumonia, at least 1 risk factor for poor outcome from COVID-19, and required oxygen supplementation without mechanical ventilation). At the time of their selection for the untreated group, the clinical outcomes of these patients were not known.

### Study Assessments

All laboratory tests and radiologic assessments were performed at the discretion of the treating physician and per standard clinical management processes. Vital signs were monitored before and upon completion of each lenzilumab infusion. Demographics, co-existing conditions, laboratory and radiographic data, as well as clinical data, adverse events, and outcomes were captured from the electronic health record until discharge or death. Similarly, for lenzilumab treated patients, data was collected up to the date of discharge or death. For untreated patients, baseline was considered their first day of hospitalization. Baseline values for the lenzilumab treated group were defined as those values obtained prior to lenzilumab administration, either on the day of administration for patients who receive lenzilumab on the first day of hospital admission or the day before the administration for patients that received lenzilumab after the first day of admission. Cytokine analysis was performed on available serum isolated from patients, pre and post lenzilumab treatment. Serum was diluted 1:2 with human serum matrix before following the manufacturer's protocol for Milliplex Human Cytokine/Chemokine MAGNETIC BEAD Premixed 38 Plex Kit (Millipore Sigma, Ontario, Canada). Data were collected using a Luminex (Millipore Sigma, Ontario, Canada).

### Statistical Methods

Continuous variables at baseline are represented using the median and interquartile range (IQR) and compared using a Wilcoxon rank-sum test. Proportions between groups at baseline were compared using Fischer's exact test. An 8-point ordinal outcome scale was used to define clinical status: 1) Death; 2) Hospitalized, on invasive mechanical ventilation or extracorporeal membrane oxygenation (ECMO); 3) Hospitalized, on non-invasive ventilation or high flow oxygen devices; 4) Hospitalized, requiring supplemental oxygen; 5) Hospitalized, not requiring supplemental oxygen—requiring ongoing medical care (COVID-19 related or otherwise); 6) Hospitalized, not requiring supplemental oxygen—no longer requires ongoing medical care; 7) Not hospitalized, limitation of activities; 8) Not hospitalized, no limitations of activities (as recommended by the WHO R&D Blueprint Group), “WHO R&D Blueprint: novel Coronavirus, COVID-19 Therapeutic Trial Synopsis,” which is incorporated herein by reference in its entirety. Clinical improvement was defined as improvement of at least two points on the 8-point ordinal scale, with the main outcome for the observation designated as the time to clinical improvement. Statistical significance for differences in temperature, serum CRP concentration, serum IL-6 concentration, absolute lymphocyte counts (ALC), and platelet counts from baseline versus 4 days post-treatment was determined using a paired t-test. Day 4 was determined as the last value for statistical analysis as data post day 4 were not available for more than 50% of this cohort. For the untreated cohort, first day of hospitalization was used as baseline and day 4 of hospitalization as the relevant time period to measure change from baseline. Differences in mean change between lenzilumab-treated and untreated groups were assessed for statistical significance with an independent two-sample t-test comparing baseline and last values as defined above. Differences in mean SpO2/FiO2 ratio over time between the treated and untreated groups was assessed using repeated measures ANOVA test. Proportion of patients with ARDS (SpO2/FiO2<315) over time between lenzilumab treated and untreated groups was assessed using repeated measures ANOVA test. Significance of proportional changes between groups was assessed by calculating the odds ratio. The time to event analyses was portrayed by Kaplan-Meier plots, and curves were compared with a log-rank test. GraphPad Prism version 8.0.0 for Windows was used to perform analysis (GraphPad Software, San Diego, Calif. USA)

### Results

### Patients and Baseline Characteristics

Twelve patients received full treatment with 3 doses of lenzilumab administered 8 hours apart. Twenty-seven patients comprised the matched control cohort. The baseline demographic and clinical characteristics of lenzilumab treated and untreated patients are summarized in Table 8.

TABLE 8
Demographics and baseline characteristics

| | Lenzilumab group | | Control group | | |
|---|---|---|---|---|---|
| Characteristic | (n = 12) | | (n = 27) | | P-value |
| Age, y | 65 | (52-70) | 68 | (61-76) | 25 |
| Male | 8 | (67%) | 19 | (70%) | >.99 |
| Female | 4 | (33%) | 8 | (30%) | >.99 |
| Race | | | | | |
| White | 9 | (75%) | 17 | (63%) | .79 |
| Asian | 2 | (17%) | 5 | (19%) | >.99 |
| American Indian/Native | 1 | (8%) | 0 | (0%) | .36 |
| American | | | | | |
| Comorbidities | | | | | |
| Diabetes mellitus | 7 | (58%) | 14 | (52%) | >.99 |
| Hypertension | 7 | (58%) | na | | na |
| Obesity (BMI >30) | 6 | (50%) | 9 | (33%) | .54 |
| Coronary artery disease | 2 | (17%) | 4 | (15%) | >.99 |
| Kidney transplantation | 1 | (8%) | na | | na |
| Obstructive lung disease | 4 | (33%) | na | | na |
| Chronic obstructive pulmonary | 2 | (17%) | 11 | (41%) | .47 |
| disease | | | | | |
| Reactive airway disease | 1 | (8%) | na | | na |
| Temperature (degrees Celsius) | 38 | (37.25-38.5) | 37.5 | (37.1-38.4) | .76 |
| Inflammatory markers before | | | | | |
| treatment | | | | | |
| CRP (<=8.0 mg/L) | 103.2 | (52.7-159.9) | 74.4 | (42.2-131.5) | .25 |
| Ferritin (24-336 mcg/L) | 596.0 | (358.3-709.0) | 673.0 | (406.8-1012.8) | .75 |
| IL-6 (<=1.8 pg/mL) | 30.95 | (24.18-34.05) | 29.20 | (13.55-40.70) | .87 |
| D-dimer (<=500 ng/mL) | 829 | (513.5-1298.5) | 916.0 | (585.0-1299.0) | .84 |
| Lymphocyte count before | 0.75 | (0.55-1.04) | 0.76 | (0.59-1.01) | .91 |
| treatment (0.95-3.07 × 10\hat{ }9/L) | | | | | |
| Oxygen therapy before treatment | | | | | |
| Nasal cannula (=4 clinical ordinal | 8 | (67%) | 20 | (74%) | >.99 |
| endpoint scale) | | | | | |
| High-flow oxygen/NIPPV (=3 | 4 | (33%) | 7 | (26%) | .73 |
| clinical ordinal endpoint scale) | | | | | |
| Invasive ventilation (=2 clinical | 0 | (0%) | 0 | (0%) | >.99 |
| ordinal endpoint scale) | | | | | |
| SpO2/FiO2 before treatment | 280.9 | (252.5-317.9) | 289.1 | (254.9-342.0) | .98 |

In the lenzilumab group, 5 (42%) patients received other pharmacotherapies targeting COVID-19 besides lenzilumab. Three patients received hydroxychloroquine, 1 of these also received tocilizumab, an IL-6 inhibitor; 1 patient each received remdesivir or systemic steroids. Among the untreated cohort, 20/27 (74%) received COVID directed therapies; 5 of these patients received more than 1 modality of treatment. Three patients received hydroxychloroquine with azithromycin, 7 patients received systemic corticosteroids, 4 patients each received tocilizumab or remdesivir, 1 patient each received ritonavir boosted lopinavir or ribavirin.

At baseline, all patients, lenzilumab treated and untreated, required oxygen supplementation, but not mechanical ventilation. In the lenzilumab group, one patient was on non-invasive positive-pressure ventilation (NIPPV), 8 (67%) were on low flow oxygen, 3 (25%) were on high flow oxygen. Among untreated patients, 2 (7.4%) were on NIPPV, 20 (74%) were on low flow oxygen and 5 (18.5%) were on high flow oxygen at baseline. In the lenzilumab group, the median SpO2/FiO2 ratio was 281, with SpO2/FiO2 ratios below 315 in 8 (67%) patients, and below 235 in 3 (25%) patients. In the untreated group, baseline median SpO2/FiO2 was 289.1, with SpO2/FiO2 ratios below 315 in 15 (56%) patients and below 235 in 6 (22%) patients. Additionally, 6 (50%) patients were febrile within 24-48 hours prior to lenzilumab administration, with a median temperature of 38.3° C. Nine (33.3%) untreated patients were febrile at baseline with a median temperature of 38.8° C.

Seven (58%) lenzilumab treated and 19 (70.3%) untreated patients had lymphopenia at baseline, with an absolute lymphocyte count less than 0.95×109/L. Median lymphocyte count before treatment was 0.75 and 0.76 in the treated and untreated groups, respectively (P=0.91). All lenzilumab patients and 26 (96%) untreated patients had an elevation in at least one inflammatory marker at baseline. Eleven (92%) treated patients had elevated CRP values above the upper limit of normal (>8.0 mg/L), with a median of 103.2 mg/L. Baseline CRP values were available for 17 (63%) of patients among the untreated group, all of which were above the upper limit of normal, with a median of 74.4 mg/L. All 11 patients in the lenzilumab group with IL-6 levels available at baseline had elevated values above the upper limit of normal (>1.8 pg/mL), with a median of 30.95 pg/mL. Similarly, all 7 patients in the untreated cohort with IL-6 levels available at baseline had elevation of IL-6, with a median of 29.2 pg/mL. Ten (83%) patients in the lenzilumab group had elevated ferritin values above the upper limit of normal (>336 mcg/L), with a median of 596 mcg/L, compared to twelve of fourteen (86%) untreated patients with available ferritin levels, with a median of 673 mcg/L. Of the 11 patients in the lenzilumab group with D-dimer levels available at baseline, 9 (75%) had values above the upper limit of normal (>500 ng/mL), with a median of 829 ng/mL. Of the 13 untreated patients with D-dimer levels available at baseline, eleven (85%) had elevated levels, with a median of 916 ng/mL (P=0.84).

### Clinical Outcomes

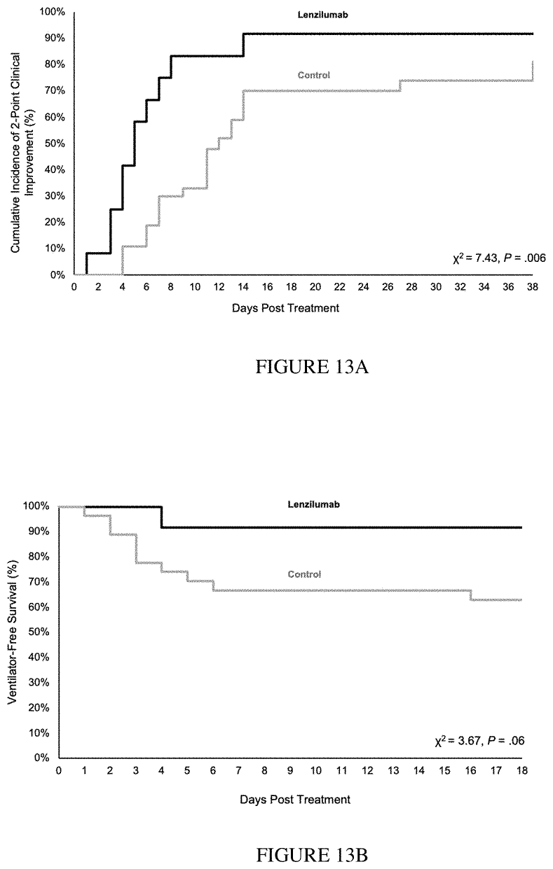

The proportion of patients who achieved clinical improvement, defined as improvement of at least 2 points on the 8-point ordinal clinical endpoints scale, was comparable in both groups: 11 out of 12 (92%) patients in the lenzilumab group and 22 out of 27 (78%) patients in the untreated group (P=0.43; Table 9). However, the time to clinical improvement was significantly shorter for patients who received lenzilumab compared to the untreated group (median 5 days [range 1-14] vs 11 days [range 4-42], x²=7.43, P=0.006; FIG. 13A). The median length of hospital stay following lenzilumab administration was significantly shorter than the median length of hospital stay for patients in the untreated group (5 days [range 3-19] vs. 11 days [range 4-38], P=0.008; Table 9).

TABLE 9
Clinical Outcomes

| | Lenzilumab group | | Control group | | |
|---|---|---|---|---|---|
| | (n = 12) | | (n = 27) | | P-value |
| Incidence of clinical | 11 | (92%) | 22 | (81%) | .43 |
| improvement | | | | | |
| Days to clinical | 5 | (1-14) | 11 | (4-42) | .006 |
| improvement | | | | | |
| Days to discharge from | 5 | (3-19) | 11 | (4-42) | .008 |
| hospital | | | | | |
| Mean temperature reduction | 1.075 | | 0.459 | | .02 |
| Days to resolution of fever | 2 | (1-6) | 1 | (1-3) | .22 |
| Incidence of IMV | 1 | (8%) | 10 | (37%) | .10 |
| Incidence of death | 1 | (8%) | 5 | (19%) | .43 |
| Incidence of IMV and/or | 1 | (8%) | 11 | (41%) | .07 |
| death | | | | | |

Ventilator-free survival favored the lenzilumab cohort compared to untreated group (x²=3.67, P=0.06; FIG. 13B). Only one (8%) patient in the lenzilumab group progressed to mechanical ventilation and death. In comparison, 10 (37%) patients in the untreated group progressed to mechanical ventilation, and 5 (19%) patients died (P=0.10 and P=0.43, respectively; Table 9).

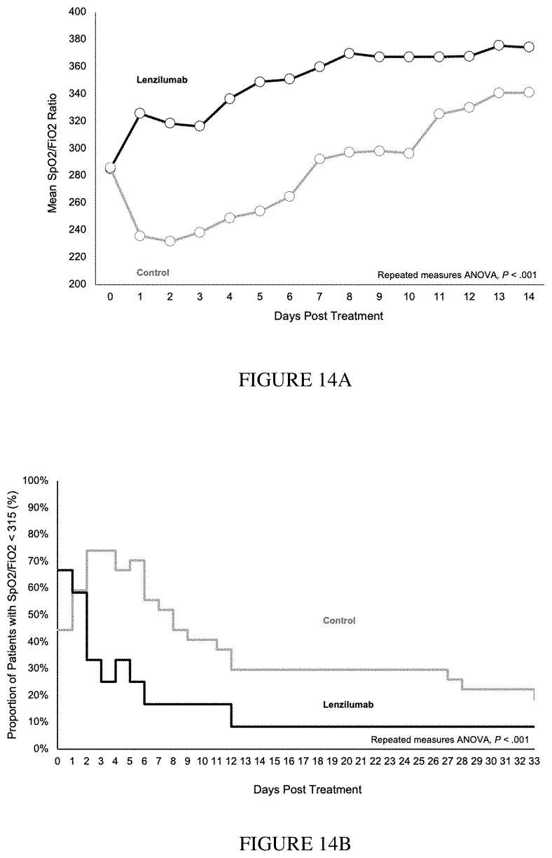

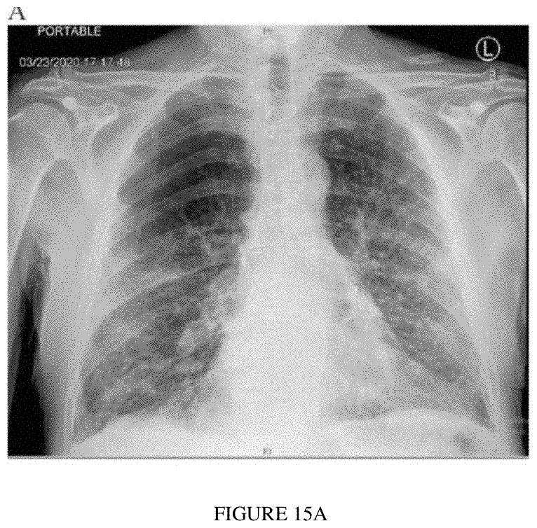

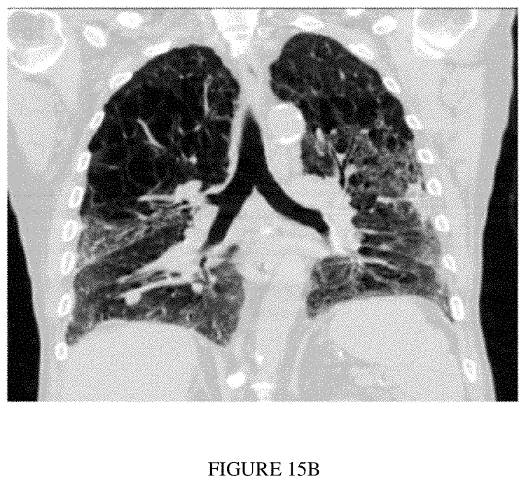

Mean baseline SpO2/FiO2 were comparable between the lenzilumab group and untreated group (285.0 vs 285.7, P=0.98). However, there was a statistically significant difference in mean SpO2/FiO2 between the lenzilumab and untreated groups over time post-treatment (P<0.001; FIG. 14A). The proportion of patients free of ARDS (who achieved a SpO2/FiO2 of 315 mmHg or higher) by the end of observation was comparable between the 2 groups: 11 (92%) patients in the lenzilumab group had achieved a SpO2/FiO2 of 315 mmHg or higher, compared with 22 (81%) patients in the untreated group (P=0.43). However, the proportion of patients free of ARDS (with SpO2/FiO2 of 315 or higher) was significantly increased in the lenzilumab group over time compared to untreated (P<0.001; FIG. 14B).

### Laboratory Markers

Baseline and follow up values that would allow comparative analysis were available for the following laboratory markers for both lenzilumab treated and untreated groups: CRP, absolute lymphocyte counts, and platelet counts. Baseline and follow-up values of IL-6 were available only for patients who received lenzilumab.

The lenzilumab group demonstrated significant reductions in mean CRP values compared to baseline (172.2 mg/L vs. 36.4 mg/L, P=0.04). A reduction of at least 50% was observed in mean CRP levels in 6 patients (50%) by day 2. In contrast, the untreated group did not have a significant reduction in mean CRP (120.6 mg/L vs. 121.7 mg/L, P=0.98). The reduction in mean CRP after 4 days of treatment was significantly greater in the lenzilumab group than in the untreated group (mean CRP reduction 135.8 vs. −0.95; P=0.01; Table 10).

TABLE 10
Laboratory Markers

| | Lenzilumab group | Control group | |
|---|---|---|---|
| | (n = 12) | (n = 27) | P-value |
| CRP reduction | 135.8 | −0.95 | .01 |
| IL-6 reduction | 20.1 | na | na |
| ALC increase | 0.46 × 10*9/L | 0.03 × 10\hat{ }9/L | .04 |
| PLT increase | 52.5 | 63.2 | .61 |

Increase in mean absolute lymphocyte counts was significantly greater among the lenzilumab treated cohort compared to the untreated group: 0.46×109/L versus 0.03×109/L, P=0.04; Table 10). Significant increases in mean platelet count from baseline were noted among both treated and untreated groups; 52.5, P=0.002 and 63.2, P<0.001, respectively. However, the difference between the two groups was not statistically significant (P=0.61, Table 10).

Compared to baseline, there was a decrease in IL-6 concentration on day 4 following lenzilumab administration: 28.6 pg/mL vs. 8.52 pg/mL, P=0.02). A decrease of at least 50% was observed in IL-6 values in 4 lenzilumab-treated patients (33.3%) by day 4.

Analysis of human cytokines comparing pretreatment with 48 hours post-lenzilumab treatment in one patient revealed significant reduction in multiple cytokines and chemokines involved in the cytokine storm (granulocyte colony-stimulating factor (G-CSF), macrophage-derived chemokine (MDC), GM-CSF, IL-1α, IFN-γ, IL-7, fms-related tyrosine kinase 3 ligand (FLT-3L), IL-1rα, IL-6, IL-12p70, FIG. 7E.

### Safety of Lenzilumab Treatment

Lenzilumab was well-tolerated in all patients. One patient, with a history of restless leg syndrome, reported a “pins and needles” sensation during the first dose of lenzilumab; those symptoms resolved and did not recur with subsequent infusions of lenzilumab. There was no significant difference in mean absolute neutrophil count or hemoglobin values between baseline and day 4 post lenzilumab: 5.1×109/L vs. 4.8×109/L, P=0.27; 12.9 g/dL vs. 11.4 g/dL, P=0.89; respectively. In one patient, hemoglobin values dropped from 10.3 g/dL on day 0 to 7.9 g/dL on day 6. This patient had undergone a renal biopsy on day 2; imaging revealed a subcapsular hematoma. At the last study observation, the patient remained anemic at 9.3 g/dL. No treatment-emergent adverse events attributable to lenzilumab were noted.

### Discussion

There is no therapy with proven efficacy against COVID-19 at present. Based on the pathophysiology of immune hyper-response following SARS-CoV-2 infection, along with prior preclinical work, it was hypothesized that lenzilumab-induced GM-CSF depletion prevents immune hyperstimulation in COVID-19 and progression to severe disease or death. The observations from the first-ever use of lenzilumab to neutralize GM-CSF in the treatment of COVID-19 are reported here. Lenzilumab was offered through a compassionate single-use IND to patients with severe and critical COVID-19 pneumonia. To provide further context for the observations, outcomes noted in the patients who received lenzilumab were compared with that of a cohort of patients hospitalized with COVID-19 pneumonia and who matched the lenzilumab patients in gender and age as well as being comparable in requiring oxygen supplementation but not mechanical ventilation and having at least 1 risk factor associated with poor COVID-19 outcomes.

The primary clinical outcome was time to clinical improvement, with clinical improvement defined as at least a 2-point improvement in the 8-point ordinal scale. In this group of high-risk patients with severe COVID-19 pneumonia, treatment with lenzilumab was associated with a significantly shorter time to clinical improvement compared to the matched cohort. Improvement in oxygen requirement was noted among lenzilumab treated as well as untreated patients. However, the proportion of patients free of ARDS (SpO2/FiO2 of 315 or higher) was significantly greater in the lenzilumab group over multiple time points. Ventilator-free survival favored the lenzilumab cohort. Among patients in the lenzilumab group, improvement in clinical parameters was accompanied by significant improvement in inflammatory markers and markers of disease severity. This was not observed for patients in the untreated group. The reduction in mean CRP in the lenzilumab group was significantly greater than in the untreated group; increases in mean absolute lymphocyte count were statistically significant in patients who received lenzilumab, but not in the untreated control group. GM-CSF depletion has been shown to result in modulation of apoptosis pathways in T cells. It is unclear at this time if the increase in lymphocyte count is secondary to clearance of SARS-CoV-2 virus, overall improvement of inflammation, or a direct effect of GM-CSF on T cells. A significant improvement in platelet count was noted both among lenzilumab treated and untreated patients. This may reflect an overall improved coagulopathy associated with COVID-19. Significant improvement in mean IL-6 was also noted following lenzilumab administration. These results are consistent with the original hypothesis described above, and corroborate the laboratory findings following GM-CSF depletion in preclinical models of CRS after CART cell therapy. FIG. 3 depicts a proposed mechanism for the role of GM-CSF in CRS post-COVID-19: SARS-CoV-2 infects monocytes/macrophages directly via the ACE-2 receptors and through antibody dependent enhancement. Infection with SARS-CoV-2 induces a T cell response through the activation of ThGM and Th17 cells. GM-CSF production by ThGM cells further stimulated monocytes and initiates an immune hyperinflammatory response. Activated monocytes result in production of myeloid derived cytokines, propagation of cytokine storm, trafficking of blood derived monocytes to the lungs, ARDS, and respiratory failure. GM-CSF activated monocytes induce T cell death and result in lymphopenia and worse clinical outcomes.

Targeting individual cytokines downstream in the inflammatory cascade of CRS, such as IL-6, have not demonstrated improved clinical outcomes in COVID-19. However, the clinical benefit observed with broad immunosuppression with dexamethasone suggests that a hyperinflammatory immune response is pathologic in latter stages of COVID-19. Neutralization of GM-CSF, which is upstream in the CRS cascade, may provide better suppression of the hyperinflammatory immune response than IL-6 receptor antagonists alone while sparing the lymphocytic effects of broad immunosuppression with steroids.

Several patients, 5 in the lenzilumab group and 20 in the untreated group, received other pharmacotherapies targeting COVID-19. These treatment decisions were not done systematically and the number of patients who received each individual therapy is so small that any meaningful analysis of their potential contribution to patients' outcomes cannot be made.

The use of lenzilumab was safe, without any adverse events attributable to lenzilumab. Numerically, more patients in the matched cohort required mechanical ventilation or died compared to patients receiving lenzilumab. However, this was not statistically significant. While there is a theoretical concern for bone marrow toxicity when GM-CSF is depleted, lenzilumab treatment was not associated with any hematological toxicity in this cohort. There were no infusion reactions following lenzilumab treatment.

The present report has several limitations. First, the sample size is small. Second, as lenzilumab was offered under emergency single-use IND conditions, all management decisions, including prescribing medications and laboratory/radiologic monitoring, were at the discretion of the treating clinicians. There was heterogeneity in the treatment specifics of individual patients as well as the laboratory and other diagnostic data that were collected. Though an attempt to provide context to the observations herein has been made by including a matched cohort, this is not a randomized controlled clinical trial. Therefore, it cannot, with full confidence, be declared that all of the clinical improvement that was observed in the patients was clearly and solely attributable to lenzilumab. However, the better outcomes in patients who received lenzilumab compared to patients in the matched cohort are very encouraging and will be further addressed in the upcoming randomized National Institutes of Allergy and Infectious Diseases (NIAID) sponsored Big Effect Trial (BET) in addition to the Phase III clinical trial (NCT04314843) that has recently been initiated.

In summary, lenzilumab was administered, under a single-use emergency IND compassionate program, to 12 patients with severe COVID-19 pneumonia and with risk factors for disease progression. Lenzilumab use was associated with faster improvement in clinical status and oxygenation, as well as greater reductions in inflammatory markers and markers of severity compared to the matched cohort. Lenzilumab was well tolerated; no treatment-emergent adverse events attributable to lenzilumab were observed.

All references cited are hereby incorporated by reference in their entirety.

Having described specific embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

### Example 11

### COVID-19 Associated Chronic ARDS Successfully Treated with Lenzilumab

Myeloid hyperinflammation leading to T-cell immune suppression and lymphocytopenia is a hallmark of severe COVID-19. Granulocyte macrophage-colony stimulating factor (GM-CSF) neutralization may prevent myeloid driven T-cell suppression leading to increased lymphocyte counts in patients with COVID-19. Given the dual mechanism of action, lenzilumab (anti-human GM-CSF monoclonal antibody) may reduce myeloid driven hyperinflammation and improve CD8+ antiviral T-cell responses directed at SARS-Cov-2 reducing the morbidity, mortality, need for invasive mechanical ventilation (IMV) and duration of hospitalization.

### Methods

Hospitalized subject with confirmed COVID-19 pneumonia and established risk factors for poor outcomes was treated in the ICU for 12 weeks using standard supportive care for chronic acute respiratory distress syndrome (ARDS). An emergency single-use investigational new drug application (IND) was approved for lenzilumab 600 tug, administered intravenously every eight hours for a total of three doses. Patient characteristics, clinical and laboratory outcomes, and adverse events were recorded through duration of hospitalization.

A 77-year-old Caucasian male presented to the emergency department (ED) who complained of difficulty breathing for the prior four days. Associated symptoms included intermittent fever of 102° F. (39.9° C.) and chills for the prior seven days. The patient's past medical history included severe chronic obstructive pulmonary disease (COPD) with emphysema, coronary artery disease with coronary artery bypass graft, systolic heart failure, type II diabetes and obstructive sleep apnea. The patient reported wearing his continuous positive airway pressure (CPAP) at night with an increase in his home oxygen use from as needed to 3 liters (L) (per minute) continuous for the past several days. The patient tested positive for severe acute respiratory, syndrome coronavirus 2 (SARS-CoV-2) and was admitted to the ICU on respiratory isolation.

On examination the patient's vital signs were: pulse of 105 bpm, respiratory rate of 20 breaths/min, blood pressure of 98/59 mmHg, oxygen saturation of 89% on 3 L of oxygen, and an oral temperature of 98.7° F. He appeared in mild distress with evidence of accessory respiratory muscle use, awake, alert and with normal appearing skin color. Cardiac auscultation noted an irregular heart rate and rhythm with a mild systolic ejection murmur and pulmonary auscultation noted diminished breath sounds at the bases with scant expiratory wheezes throughout. Per the ED workup, the patient had atrial fibrillation on electrocardiogram, evidence of bilateral infiltrates on chest x-ray (FIG. 15A), lymphopenia on complete blood count (CBC) and a mild transaminitis on liver function tests.

At the time of admission in March 2020, treatment options for COVID-19 were limited. The patient was started on broad spectrum antibiotics for community acquired pneumonia, steroids, and bronchodilators for possible COPD exacerbation and hydroxychloroquine with zinc for COVID-19. He continued to deteriorate for the next 12 weeks with an increase in oxygen demand from continuous low-flow oxygen to high-flow and eventually intermittent bilevel positive airway pressure (BIPAP). Given his degree of severe hypoxemia, inhaled epoprostenol was added with marginal improvement of his alveolar-arterial gradient. The patient's chest computed tomography scan (CT) was significant for diffuse ground glass opacities predominately at the bases with bilateral upper lob severe emphysematous changes (FIG. 15B). Echocardiogram demonstrated left ventricular ejection fraction of 40-45% and mild mitral regurgitation. The initial sputum culture was positive for Stenotrophomonas maltophilia for which he was placed on trimethoprim/sulfamethoxazole (TMP/SMX). He eventually underwent flexible bronchoscopy due to persistent hypoxemia which resulted in positive candida cultures and fluconazole was initiated. Antimicrobial therapy was tailored for sensitivities and course duration by an infectious disease specialist.

Over the course of his ICU stay, the patient developed acute respiratory distress syndrome (ARDS). On week 11, and after several unsuccessful attempts at oxygen weaning, the patient was considered for lenzilumab (Humaneered® anti-hGM-CSF monoclonal antibody), a novel COVID19 therapy, given the results in recent positive case-control report Examples 8 and 10.

An emergency single use IND was approved on week 13 and after the patient was consented, lenzilumab was administered at a dose of 600 mg intravenously every 8 hours, for a total of 3 doses. No infusion-related or systemic side effects were noted.

### Results

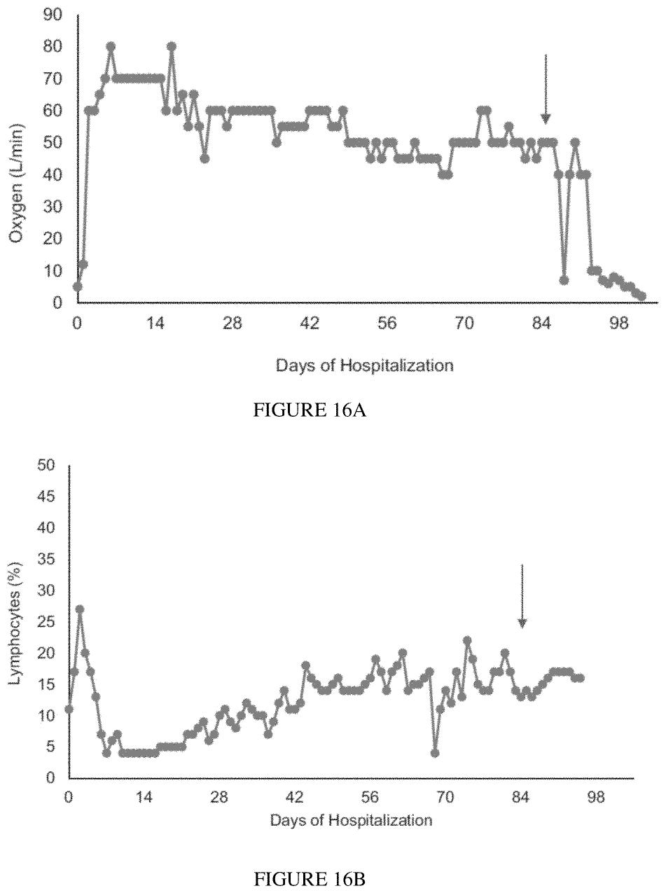

One-week post lenzilumab therapy, the patient's oxygen demand decreased from high-flow to low-flow nasal cannula and he was able to walk with physical therapy outside of his room (FIG. 16A). The patient's lymphopenia, which had been slowly improving, appeared to improve post lenzilumab therapy (FIG. 16B). Sixteen days post lenzilumab (week 15), the patient was discharged home on 4 L nasal cannula.

A 77-year-old Caucasian male with past medical history of severe chronic obstructive pulmonary disease (COPD) with emphysema, coronary artery disease, type II diabetes, and obstructive sleep apnea was admitted to ICU with fever, shortness of breath and confirmed SARS-CoV-2 infection. The patient was treated with standard supportive care including corticosteroids. Over the course of his ICU stay, the patient developed ARDS and on week 13, and after several unsuccessful attempts at oxygen weaning, lenzilumab was administered via emergency single use IND. One-week post lenzilumab therapy, oxygen demands decreased, lymphopenia appeared to improve and sixteen days post lenzilumab therapy, the patient was discharged home on 4 L nasal cannula. No infusion-related or systemic side effects were noted.

### Conclusion

In a case of COV D-19 with multiple co-morbidities, refractory to corticosteroids, and deteriorating for several months, GM-CSF neutralization with lenzilumab appeared to reduce oxygen requirements, improve lymphopenia and accelerate time to recovery/discharge in a COVID-19 subject. A randomized, double-blind, placebo-controlled phase 3 clinical trial is ongoing to validate these findings (NCT04351152).

### DISCUSSION

Advanced age, male sex, COPD, and type II diabetes are all associated risk factors for severe COVID-19. This patient presented with respiratory symptoms and upon examination significant radiographic abnormalities were found. The patient progressed to ARDS that did not resolve with standard therapies including steroids. Recent clinic data suggests that steroids may only be appropriate for patients on invasive mechanical ventilation with high levels of c-reactive protein (CRP) and non-diabetics while those patients with lower CRP levels and diabetes may be harmed by steroid use.

The patient's length of hospitalization (15 weeks) is highly unusual as it is currently estimated that 95% of COVID-19 associated hospitalizations last between 1 and 31 days. Extended length of stay coupled with lymphopenia increases the risk of hospital acquired infection, as demonstrated in the patient's susceptibility to bacterial and fungal infection.

Recent immune-profiling studies of patients with severe COVID-19 suggest a myeloid driven hyperinflammatory immune suppression as the underlying pathophysiology wherein immature and dysfunction myeloid cells result in inflammation but also profound suppression of T-cell responses delaying viral clearance and increasing susceptibility to opportunistic infections. Neutralization of GM-CSF may suppress myeloid hyperinflammation and restore balance to the dysregulated immune response. Lenzilumab is currently being studied in a phase III trial for severe and critical COVID-19 pneumonia (NCT04351152) and in the National Institute of Allergy and Infectious Diseases (MAID) sponsored Big Effect Trial (BET) in combination with remdesivir.

After deterioration in the hospital for 13 weeks, this patient was administered lenzilumab under an emergency single use IND. Rapid resolution of the patient's hypoxemia was demonstrated by a reduction in oxygen requirements, improved mobility, and accelerated time to discharge. A recent case-control study (Example 10) suggests lenzilumab may improve clinical outcomes, oxygenation requirements, and improve lymphocyte counts in patients with severe and critical COVID-19 during the acute hyperinflammatory immune response. The present case report suggests that lenzilumab may be beneficial to patients who are unable to wean off of supplemental oxygen, have failed multiple rounds of prior therapy, and are outside the initial acute hyperinflammatory window.

### Example 12

### Lenzilumab™ Improves Survival without Need for Mechanical Ventilation in Hospitalized Patients with COVID-19

This study was a multi-center, randomized, double-blind, placebo-controlled Phase 3 trial for the treatment and prevention of serious and potentially fatal outcomes in patients who were hospitalized with COVID-19 pneumonia. The primary objective was to assess whether lenzilumab, in addition to existing standard of care, which included dexamethasone (or other steroids) and/or remdesivir, could alleviate the immune-mediated cytokine release syndrome (CRS) and improve ventilator-free survival. Ventilator-free survival is a composite endpoint of time to death and time to invasive mechanical ventilation (IMV), which is a robust measure that is less prone to favor a treatment with discordant effects on survival or days free of ventilation. The trial enrolled 520 patients in 29 sites in the US and Brazil who were at least 18 years of age; experienced blood oxygen saturation (SpO2) of less than or equal to 94%; or required low-flow supplemental oxygen, or high-flow oxygen support, or non-invasive positive pressure ventilation (NIPPV); and were hospitalized but did not require IMV. Following enrollment, subjects were randomized to receive three infusions of either 600 mg of lenzilumab or placebo, each infusion separated by eight hours over a 24 hour period, along with concomitant existing standard of care. Standard of care included steroids (dexamethasone) and/or remdesivir. The primary endpoint was the difference between lenzilumab treatment and placebo treatment in ventilator-free survival through 28 days following treatment. Key secondary endpoints, also measured through 28 days, included ventilator-free days, duration of intensive care unit (ICU) stay, incidence of invasive mechanical ventilation, extracorporeal membrane oxygenation (ECMO), and/or death, time to death, all-cause mortality, and time to recovery.

The study enrolled 520 patients in 29 sites in the US and Brazil who were at least 18 years of age; experienced blood oxygen saturation (SpO₂) of less than or equal to 94%; or required low-flow supplemental oxygen, or high-flow oxygen support, or non-invasive positive pressure ventilation (NIPPV); were confirmed to have tested for SARS-CoV-2 COVID-9 pneumonia; and were hospitalized but did not require IMV.

Following enrollment, subjects were randomized to receive three infusions of either lenzilumab or placebo, each infusion separated by eight hours over a 24-hour period with other treatments. The actual dosing in this study was at 552 mg IV every 8 hours×3. The full course of lenzilumab (1,656 mg) is given over a 24 hour period The primary endpoint was the difference between lenzilumab treatment and placebo treatment in ventilator-free survival through 28 days following treatment. Key secondary endpoints, also measured through 28 days, included ventilator-free days, duration of ICU stay, incidence of invasive mechanical ventilation, extracorporeal membrane oxygenation (ECMO), and/or death, time to death, all-cause mortality, and time to recovery. Results of the trial are planned to be submitted for potential publication in a peer-reviewed journal. All subjects received concomitant treatment, including corticosteroids, remdesivir or both. Approximately 88% of patients received dexamethasone (or other steroids), 62% received remdesivir, and 57% received both, balanced across both arms of the study.

The study incorporated a diverse population with various comorbidities, most commonly a body mass index above 30, which is representative of a real-world, high-risk population Table 11 shows the demographics and baseline characteristics (mITT population).

The present study used a modified ITT (mITT) analysis that excluded 33 patients—19 in the active arm and 14 in the placebo cohort. Table 11 shows the demographics of the mITT population. As used herein, the definition of “severe” for this trial was SpO2<=94% or needing low-flow oxygen support, while “critical” was defined as the need for high-flow oxygen support or a non-invasive positive pressure device.

Demographics and Baseline Characteristics (mITT Population)

| | Lenzilumab | | Placebo | | Overall | |
|---|---|---|---|---|---|---|
| Characteristics | (N = 236) | | (N = 243) | | (N = 479) | |
| Age | | | | | | |
| Mean (SD) | 60.5 | (13.5) | 60.5 | (14.3) | 60.5 | (13.9) |
| Median (Min-Max) | 62.0 | (28-98) | 62.0 | (22-96) | 62.0 | (22-98) |
| <65 years old (%) | 60.2 | | 58.4 | | 59.3 | |
| ≥65 years old (%) | 39.8 | | 41.6 | | 40.7 | |
| Gender | | | | | | |
| Male (%) | 64.8 | | 64.6 | | 64.7 | |
| Ethnicity | | | | | | |
| Hispanic or Latino (%) | 35.2 | | 42.0 | | 38.6 | |
| Not Hispanic or Latino (%) | 64.0 | | 56.8 | | 60.3 | |
| Race | | | | | | |
| White (%) | 69.9 | | 73.3 | | 71.8 | |
| Black or African American (%) | 16.1 | | 13.6 | | 14.8 | |
| Asian (%) | 4.2 | | 2.1 | | 3.1 | |
| American Indian/Alaska Native (%) | 1.7 | | 0.0 | | 0.8 | |
| Other (%) | 8.1 | | 11.0 | | 9.7 | |
| Body Mass Index | | | | | | |
| Median (Min-Max) | 31.5 | (20.3-75.5) | 30.5 | (18.3-75.2) | 31.1 | (18.3-75.5) |

| | | | |
|---|---|---|---|
| ≥30 Kg/m² (%) | 57.6 | 52.7 | 55.1 |
| Clinical Status at Baseline | | | |
| SpO₂ ≤94% or low-flow oxygen (%) | 61.9 | 57.6 | 59.7 |
| High-flow oxygen or NIPPV (%) | 38.1 | 42.4 | 40.3 |

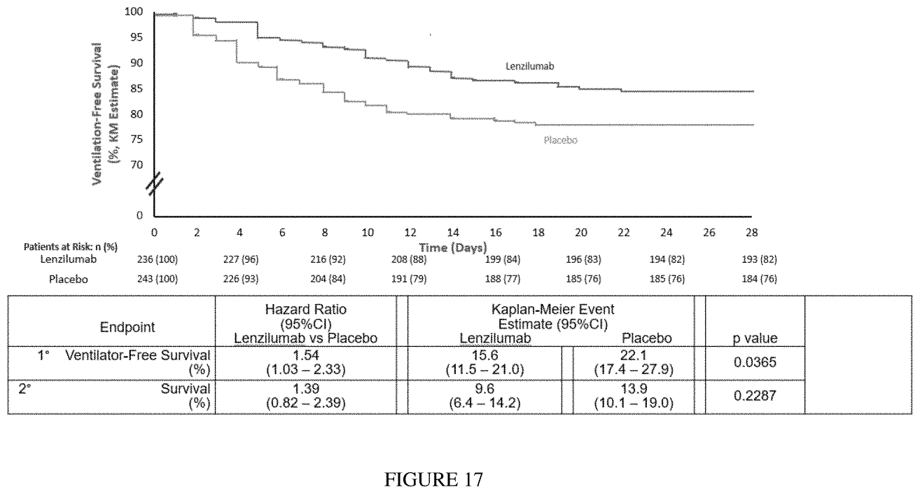

Patients were assessed through Day 28 after treatment. The primary endpoint was ventilator-free survival. See FIG. 17. Key secondary endpoints were ventilator-free days, duration of ICU stay, survival, and time to recovery.

The primary endpoint of the study was ventilator-free survival which can be found in the VFS mITT Table 12. The HR (hazard ratio) shows the improvement in ventilator free survival for each co-variate (treatment, <65 vs >65, severe vs. critical) with the 95% confidence intervals and p-value.

TABLE 12
Endpoints of VFS for mITT population.

| | Hazard Ratio | | | |
|---|---|---|---|---|
| | (95% CI) | Kaplan-Meier Event | | |
| | Lenzilumab vs | Estimate (95% CI) | | |
| Endpoint | Placebo | Lenzilumab | Placebo | p value |
| 1° | 1.54 | 15.6 | 22.1 | 0.0365 |
| Ventilator- | (1.03-2.33) | (11.5-21.0) | (17.4-27.9) | |
| Free | | | | |
| Survival | | | | |
| (%) | | | | |
| 2° | 1.39 | 9.6 | 13.9 | 0.2287 |
| Survival | (0.82-2.39) | (6.4-14.2) | (10.1-19.0) | |
| (%) | | | | |

The study results demonstrate that lenzilumab significantly improved patient outcomes. The study achieved its primary endpoint of ventilator-free survival measured through day 28 following treatment (HR: 1.54; 95% CI: 1.03-2.33, p=0.0365). Ventilator-free survival is a validated and reliable measure used in studies that evaluate respiratory distress. The Kaplan-Meier estimate for IMV and/or death was 15.6% (95% CI: 11.5-21.0) in the lenzilumab arm versus 22.1% (95% CI: 17.4-27.9) in the placebo arm, representing a 54% improvement in the relative likelihood of survival without the need for IMV. Although this study was not powered to demonstrate a difference in mortality, a favorable trend in mortality was observed: 9.6% (95% CI: 6.4-14.2) in the lenzilumab arm compared with 13.9% (95% CI: 10.1-19.0) in the placebo arm (HR: 1.39; 95% CI: 0.82-2.39; p=0.2287). Serious adverse events (SAEs) were balanced in both study arms and the SAE profile was similar to that previously documented in prior lenzilumab studies. In this study, lenzilumab appeared to be safe and well-tolerated; no new SAEs were identified, and none were attributed to lenzilumab.

Table 13 shows safety was balanced between treatment arms, i.e., lenzilumab or placebo.

Safety: Balanced Between Treatment Arms

| | Lenzilumab (N = 255) | | Placebo (N = 257) | | Overall (N = 512) | |
|---|---|---|---|---|---|---|
| Characteristics | Subjects n, % | | Subjects n, % | | Subjects, n % | |
| Serious Adverse Events | 63 | (24.7) | 76 | (29.6) | 139 | (27.1) |
| Respiratory | 60 | (23.5) | 67 | (26.1) | 127 | (24.8) |
| Cardiac | 12 | (4.7) | 13 | (5.1) | 25 | (4.9) |
| Infection/Infestation | 9 | (3.5) | 9 | (3.5) | 18 | (3.5) |
| Vascular | 5 | (2.0) | 10 | (3.9) | 15 | (2.9) |
| General/Administration | 3 | (1.2) | 9 | (3.5) | 12 | (2.3) |
| Site | | | | | | |
| Renal and Urinary | 3 | (1.2) | 8 | (3.1) | 11 | (2.1) |
| Gastrointestinal | 1 | (0.4) | 3 | (1.2) | 4 | (0.8) |
| Nervous System | 1 | (0.4) | 3 | (1.2) | 4 | (0.8) |
| Blood and Lymphatic | 1 | (0.4) | 0 | (0) | 1 | (0.2) |

Ventilator-free survival may provide a signal of survival benefit with fewer patients In particular, ventilator-free survival is a robust measure less prone to favor a treatment with discordant effects on survival and days free of ventilation. Ventilator-free survival may act as a surrogate endpoint for survival. Hazard ratio (HR) for ventilator-free survival approximates the HR for survival in the RECOVERY studies (see recoverytrial.net). Table 14 shows a comparison of results from the RECOVERY study and the study of Example 12:

| | Ventilator-Free Survival | | | |
|---|---|---|---|---|
| | | Increased | Survival | |
| | | Likelihood | | Increased |
| | Hazard | of Survival | Hazard | Likelihood |
| Treatment | Ratio | Without IMV | Ratio | of Survival |
| RECOVERY TRIAL* | | | | |
| Dexamethasone* | 1.15 | 15% | 1.16 | 16% |
| Tocilizumab* | 1.18 | 18% | 1.14 | 14% |
| HUMANIGEN PHASE | | | | |
| 3 TRIAL | | | | |
| Lenzilumab | 1.54 (SS) | 54% | 1.39 | 39% (NS) |

SS—Statstically signficant
NS—Not statistically significant

The results from this study with lenzilumab treatment were associated with better outcomes in hospitalized hypoxic COVID-19 patients who had not yet progressed to the point of requiring IMV. The study results showed that patients who received lenzilumab and other treatments, including steroids and/or remdesivir, had a 54% greater relative likelihood of survival without the need for IMV compared with patients receiving placebo and other treatments. These results are statistically significant.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

### Example 13

### Lenzilumab™ Efficacy and Safety in Newly Hospitalized COVID-19 Subjects: Results from the LIVE-AIR Phase 3 Randomized Double-Blind Placebo Controlled Trial

LIVE-AIR (Lenzilumab Improves the Likelihood of Survival Without Ventilation in Newly Hospitalized Subjects Exploring CRP<150 mg/L Age Less than 85 Years, with active Inflammation Requiring Oxygen), a phase 3 randomized, double-blind, placebo-controlled clinical trial was designed to demonstrate that early intervention with lenzilumab, in newly hospitalized COVID-19 subjects who require supplemental oxygen but have not progressed to IMV, improves the likelihood of survival without ventilation (SWOV, sometimes referred to as ventilator-free survival) beyond that provided by supportive care with corticosteroids and remdesivir. Herein are reported the efficacy safety results following 28 days of follow-up.

### Methods

### Trial Design

The LIVE-AIR trial (NCT04351152) was a phase 3 randomized, double-blind, placebo controlled study in 520 hospitalized subjects with severe COVID-19 pneumonia across 29 sites in the U.S. and Brazil. The study was conducted in accordance with the Good Clinical Practice guidelines of the International Council for Harmonization E6 and the principles of the Declaration of Helsinki. The protocol was approved by the central/local institutional review board or ethics committee at each site.

All subjects provided written informed consent. Enrolled participants were randomized 1:1 to receive lenzilumab or matched placebo in addition to current standard treatment per institutional guidelines at each site. Subjects were stratified at randomization by age (≤65 vs. >65 years) and disease severity (oxygen saturation [SpO2]<94% on room air or requiring low-flow supplemental oxygen vs. requirement for high-flow oxygen delivery device or non-invasive positive pressure ventilation [NPPV] or multi-organ dysfunction/failure or shock).

Following screening and baseline measures, participants were administered either lenzilumab or placebo saline beginning at Day 0 within 12 hours from the time of randomization in addition to standard care in accordance with institutional site treatment guidelines. All subjects were monitored at screening, baseline just prior to administration of study drug on Day 0, and at least daily while hospitalized according to the schedule of assessments through Day 28. All primary and key secondary endpoints were assessed by Day 28; however, subjects were followed through Day 60. Adverse events subjects were graded using the National Cancer Institute (NCI) Common Terminology Criteria for Adverse Events (CTCAE) version 5.0, which grades events on a scale from 1 (the mildest intensity) to 5 (fatal). A pharmacokinetic analysis of lenzilumab was also pre-specified (data not shown).

### Subjects

Eligibility criteria for enrollment included subjects at least 18 years of age who provided informed consent, provided by an authorized proxy, if necessary. SARS-CoV-2 infection was virologically confirmed and pneumonia was diagnosed by chest x-ray or computed tomography. Subjects must have been hospitalized with SpO2≤94% on room air and/or in need of supplemental oxygen in the form of low-flow oxygen, high-flow oxygen, or NPPV.

Subjects were excluded if they required IMV or extracorporeal membrane oxygenation (ECMO), were pregnant or, in the view of the treating investigator, were not expected to survive the following forty-eight hours from the time of randomization. Subjects with a confirmed diagnosis of bacterial pneumonia or other active/uncontrolled fungal or viral infection other than SARS-CoV-2 were also excluded. Women of childbearing potential were eligible if they had a negative urine or serum pregnancy test at screening/baseline and agreed to adequate contraception following their last dose of study drug.

### Treatments

Lenzilumab or matching placebo (0.9% saline for injection) was administered by intravenous infusion beginning at Day 0 within 12 hours of randomization. Three doses of lenzilumab (600 mg, each) or placebo were administered 8 hours apart via a 1 hour IV infusion, in addition to standard supportive care. Standard pre-infusion medications comprised of acetaminophen 500 to 1000 mg PO or IV and diphenhydramine 12.5 to 25 mg IV, or 25 mg PO or equivalent, and were administered approximately 1-hour prior to infusion of study drug.

Allowed treatments, at baseline and throughout the study, included all current COVID-19 treatments: corticosteroids, convalescent plasma, remdesivir or other anti-virals, and/or hydroxychloroquine with or without azithromycin. Disallowed medications were FDA approved monoclonal antibodies targeting IL-6 or IL-1, kinase inhibitors, and COVID-19 neutralizing monoclonal antibodies if used within 8 weeks prior to randomization. Sargramostim as well as other investigational therapies to treat COVID-19 were not permitted.

### Outcomes Measures

Assessment of COVID-19 associated pneumonia for development of adult respiratory distress syndrome (ARDS) was conducted during the hospital stay through day 28. This included symptom-directed physical examination, assessment of clinical status using an 8-point ordinal scale adapted (1=death, 8=no hospitalization, no limitations) from the NIH-sponsored Adaptive COVID-19 Treatment Trial (ACTT, NCT 04280705)19, vital signs, laboratory tests, use of IMV or ECMO, non-invasive ventilation (or high-flow oxygen devices), and low-flow supplemental oxygen, ICU admission, assessment of key cytokines, and adverse events.

The primary efficacy endpoint was a composite endpoint of ventilator-free survival also referred herein as survival without ventilation (SWOV), by Day 28. Failure of the primary endpoint was defined as mortality or the requirement for IMV. SWOV is a robust composite endpoint that is less prone to favor treatments with discordant effects on survival and days free of ventilation20 while avoiding the need for sample sizes approaching those of mortality trials to enable timely availability of study results. Key secondary endpoints included the proportion of IMV, ECMO or death, mortality, and Time to Recovery based on the 8 point clinical status scale.

### Sample Size Estimation

The sample size estimate was based on the event rates in similar patient populations from other published studies. The event rate of subjects who required IMV or died by Day 28 in the placebo group was estimated to be approximately 25%, and the event rate in the lenzilumab treatment group was approximated as 15% resulting in a HR of 0.565.

Using a Cox proportional hazard model to test for inequality of the HR, a total of 100 events were calculated as needed to provide 81.47% power to detect a difference using a two-sided alpha of 5.0% at the final analysis and assuming a fixed follow-up of 28 days. Therefore, approximately 516 subjects (258 subjects in each treatment group) were to be enrolled to observe the 100 targeted events.

### Statistical Analysis

The primary endpoint was the difference between lenzilumab treatment and placebo treatment in SWOV through 28 days following treatment in the modified intent to treat population (mITT) who received at least one dose of investigational treatment under the documented supervision of the principal investigator or sub-investigator. This population was defined as the primary analysis and used a Cox proportional hazard model (HR:lenzilumab relative to placebo) accounting for the stratification variables (i.e., age and disease severity) supplemented by a display of K-M curves in each treatment group. The Cox proportional hazard model included the time to first event as the dependent variable (1=IMV use or death, 0=alive with no IMV use); treatment (covariate); and strata (covariates). Where data were non-proportional based on a Chi-squared test proposed by Grambsch and Therneau with a global p-value <0.05, a weighted Cox proportional hazard regression model was used to correct for non-proportionality. For sensitivity and exploratory analyses of the primary endpoint, step-wise addition of all possible two-way interactions between the three covariates was considered. The model with the best fit (lowest AIC value) was selected. Subjects who were alive and did not get placed on IMV were right censored at the date of the last non-missing assessment on or prior to Day 28.

The primary analyses were performed in the mITT and ITT populations, respectively, (1) among subjects overall and subjects who (2) had CRP<150 mg/L at screening or if no screening value on day one (baseline) and age <85, (3) received remdesivir and/or any corticosteroid, and (4) randomized <2 days from hospitalization and ≥2 days from hospitalization. The cut-off for CRP was based on the COVID-19-associated hyperinflammation phenotype (COV-HI). The age cut-off was an original protocol exclusion that was removed in later versions.

For each secondary endpoint, the proportion of subjects that had the event was calculated by treatment group. An odds ratio was calculated for the composite endpoint of the first incident IMV, ECMO or death using logistic regression and including baseline age group and disease category as covariates. Hazard ratios were calculated for each of time to death and time to recovery, separately, as described above. For time to recovery, deaths were censored at Day 28. Subjects who were alive yet did not recover were right censored at the date of the last non-missing assessment of the 8-point clinical status ordinal scale on or prior to Day 28. Last, the proportion of subjects who had CTCAE grade ≥3 treatment-emergent serious adverse events were quantified for each randomization group by system organ class. All data reported herein are reported through Day 28.

Safety was assessed on study drug received, regardless of assignment group. Eight randomized subjects were never treated and were therefore excluded from the safety analysis but were included in the ITT analyses. One subject, randomized to placebo, received lenzilumab in error and was included in the safety analysis of lenzilumab and in the mITT efficacy analysis of placebo.

### Results

### Subjects

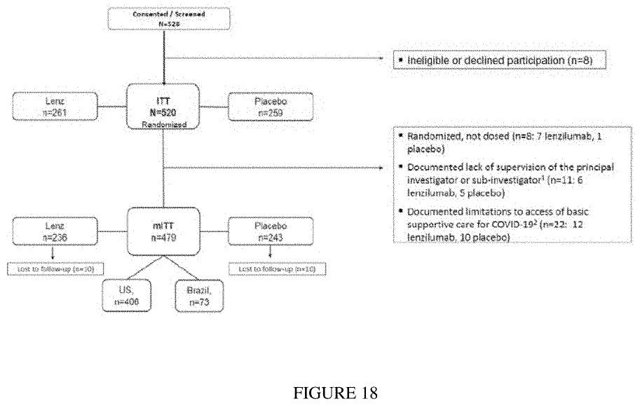

Five hundred, twenty-eight subjects were screened, of whom 520 were randomized and included in the ITT population (FIG. 18). Forty-one 41 subjects (7.9%) were excluded from the mITT population. Of these, 22 were from two sites in Brazil (12 lenzilumab; 10 placebo) that joined the final stage of the study and experienced documented limitations to access of basic supportive COVID-19 care. These sites experienced an unexpected surge in COVID-19 hospitalizations which affected their ability to provide high-flow oxygen devices, which resulted in a disproportionate increase in the escalation of care from low-flow supplemental oxygen directly to IMV. Subjects from these sites were excluded while the study remained blinded. The mITT population was 236 and 243 subjects in the lenzilumab and placebo groups, respectively: representing 90% and 94% of each randomized population. Despite the challenges, such as those listed above, of conducting a study during a pandemic, the study experienced a loss to follow-up of <2% in each arm and had robust monitoring that yielded 100% source data verification and adherence to good clinical practices.

Baseline characteristics were well-balanced between groups (Table 15). Approximately two-thirds of the subjects were male with a mean age of 60.5 years. The subjects were of diverse racial and ethnic backgrounds with 56.8% self-reported as minority or other and 43.2% reporting as white, which is consistent with real-world demographics of hospitalized subjects with COVID-19. At randomization, 40.5% of subjects were ordinal score 3 (high-flow oxygen or NPPV) and 59.5% were ordinal score 4 (low-flow supplemental oxygen) or 5 (SpO2<94% on room air) on the adapted 8-point ordinal scale.

Obesity was the most comorbidity followed by diabetes, chronic kidney disease, and coronary artery disease. Subject received corticosteroids (93.7%), remdesivir (72.4%) or both (69.1%) while also receiving study drug. Subjects were hospitalized a median of 2 days prior to randomization. Eighty-five percent of the subjects were enrolled in the U.S.

TABLE 15
Baseline Characteristics

| | Lenxilumab | Placebo | Total |
|---|---|---|---|
| | (n = 236) | (n = 234) | (n = 479) |
| Gender | | | |
| Male (%) | 64.8 | 64.6 | 64.7 |
| Age | | | |
| Mean (SD) | 60.5 [symbol] | 60.5 [symbol] | 60.5 [symbol] |
| Median (min-max) | 62.0 [symbol] | 62.0 [symbol] | 62.0 [symbol] |
| <65 (%) | 60.2 | 58.4 | 59.3 |
| [symbol] 65 (%) | 29.8 | 41.6 | 40.7 |
| >80 (%) | 7.6 | 5.3 | 6.5 |
| EMI | | | |
| Mean (SD) | 33.1 (8.4) | 31.0 (7.9) | 32.5 (8.2) |
| [symbol] (%) | 57.6 | 52.7 | 55.1 |
| Race (%) | | | |
| American Indian | 1.1 | 0.0 | 5.0 |
| Asian | 2.7 | 1.2 | 1.9 |
| Black | 10.3 | 13.7 | 8.9 |
| Hispanic | 35.2 | 42.0 | 38.6 |
| White | 45.0 | 41.6 | 43.2 |
| Other | 5.2 | 6.2 | 5.8 |
| Region (%) | | | |
| US | 66.0 | 84.8 | 85.4 |
| Brazil | 14.0 | 15.2 | 14.6 |
| [symbol] Oxygen | | | |
| Room air or Low Flow Oxygen | 61.9 | 57.2 | 59.5 |
| High Flow Oxygen or NPPV | 38.1 | 42.8 | 40.5 |
| CRP | | | |
| Mean (SD) | 100.95 (80.14) | 95.66 (71.01) | 96.36 (75.57) |
| CRP <150 mg/L (%) | 75.8 | 79.9 | 77.9 |
| CRP >150 mg/L (%) | 24.2 | 20.1 | 22.1 |
| Co-Morbidity (%) | | | |
| Cardiovascular | | | |
| [symbol] Heart Failure | 1.21 | 10.3 | 11.7 |
| Coronary Artery Disease | 14.8 | 1.23 | 13.6 |
| Diabetes | 50.8 | 56.0 | 53.4 |
| Chronic Liver Disease | 4.2 | 5.8 | 5.0 |
| Chronic Kidney Disease | 14.4 | 13.6 | 14.0 |
| Respiratory | | | |
| Asthma | 13.6 | 7.8 | 10.6 |
| [symbol] Pulmonary Fibrosis | 1.3 | 0.4 | 0.8 |
| COPD | 7.6 | 7.0 | 7.3 |
| Treatments (%) | | | |
| [symbol] | 72.1 | 72.8 | 72.4 |
| [symbol] | 93.7 | 93.8 | 93.7 |
| [symbol] | 69.1 | 69.1 | 69.1 |

[symbol]

indicates data missing or illegible when filed

### Primary Outcome

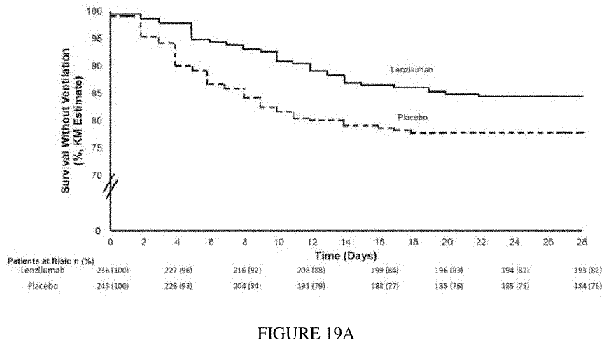

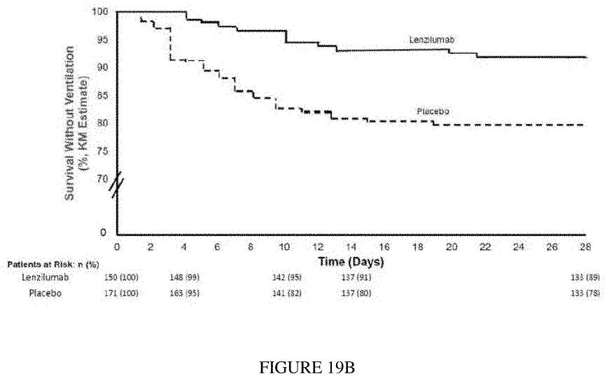

The study achieved its pre-specified primary endpoint. In the mITT population, the Cox proportional hazard model estimated a 54% relative improvement in the likelihood of subjects treated with lenzilumab achieving SWOV compared to the placebo group (HR: 1.54; 95% CI: 1.02-2.31, p=0.041; Table 16, FIG. 19A). Failure to achieve SWOV occurred in 15.6% and 22.1% of subjects treated with lenzilumab or placebo, respectively (Table 16). Separation of the survival curves occurred as early as 3 days following treatment (FIG. 19A), continued to increase through approximately Day 10, and then was maintained for the duration of the 28-day observation period. In the ITT population, lenzilumab improved the likelihood of SWOV by 1.90-fold (nominal p=0.043; Table 16).

TABLE 16
Survival Without Ventilation

| | Kaplan-Meier Estimate of | | | |
|---|---|---|---|---|
| | Failure to Achieve SWOV | | [symbol] Placebo | |
| | [symbol] | Placebo [symbol] | Hazard Ratio [symbol] | |
| Population | (95% CI) | (95% CI) | (95% CI) | P Value |
| mITT [symbol] | 15.6% | 22.1% | [symbol] | [symbol] |
| (n = 479) | (11.5-21.0) | (1.7-27.9) | | |
| | (n = 296) | (n = 243) | | |
| ITT [symbol] | 18.9% | [symbol] | 1.90 | [symbol] |
| (n = 520) | (14.5-24.3) | (n = 259) | (1.02-3.52) | |
| | [symbol] | | | |

CRP <150 mg/L and

[symbol]

| | | | | |
|---|---|---|---|---|
| mITT, | 8.5% | 21.1% | 2.95 | 0.0003 |
| (n = 336) | (5.1-14.3) | [symbol] | [symbol] | |
| | (n = 157) | (n = 179) | | |
| ITT, | 11.5% | 22.6% | [symbol] | 0.0080 |
| (n = 359) | (7.5-17.5) | (1.7-29.3) | (1.32-3.75) | |
| | (n = 169) | (n = 190) | | |

[symbol]

| | | | | |
|---|---|---|---|---|
| mITT, | 15.6% | 25.7% | [symbol] | 0.0073 |
| (n = 347) | (10.9-22.1) | [symbol] | (1.19-3.05) | |
| | (n = 170) | (n = 177) | | |
| ITT, | 17.0% | 26.5% | [symbol] | 0.0099 |
| (n = 354) | (12.2-23.6) | (20.6-33.7) | (1.15-2.84) | |
| | (n = 175) | (n = 179) | | |

[symbol]

| | | | | |
|---|---|---|---|---|
| mITT, | 16.3% | 26.9% | 1.92 | 0.0067 |
| (n = 331) | (11.4-23.1) | (20.9-34.4) | [symbol] | |
| | (n = 163) | (n = 168) | | |
| ITT, | 17.8% | 27.8% | 1.82 | 0.0092 |
| (n = 338) | (12.7-24.5) | (21.7-35.2) | (1.16-2.85) | |
| | (n = 168) | (n = 170) | | |

[symbol]

| | | | | |
|---|---|---|---|---|
| mITT, [symbol] | 16.5% | 27.6% | 1.88 | 0.015 |
| 2 days prior | (11.4-23.6) | (21.1-35.6) | (1.13-31.2) | |
| (n = 297) | (n = 149) | (n = 146) | | |
| mITT, >2 days | 13.7% | 14.4% | 0.88 | 0.762 |
| prior | (7.6-13.9) | (3.6-23.5) | (0.38-20.2) | |
| (n = 165) | (n = 74) | (n = 91) | | |

[symbol]

data observed at [symbol] days following enrollment. [symbol]
Primary endpoint [symbol]
mITT, [symbol] ITT,

[symbol]

[symbol]

[symbol]

[symbol]

[symbol]

[symbol]

indicates data missing or illegible when filed

The primary outcome was substantiated by sensitivity analysis Baseline CRP was obtained in 482 subjects, of which 77.9% had a CRP level of <150 mg/L. In these subjects who were also <85 years of age, lenzilumab improved the likelihood of SWOV by 2.96-fold (nominal p=0.0003) in the mITT analysis (Table 16, FIG. 19B). A second sensitivity analysis on the ITT population with CRP<150 mg/L and <85 years old, lenzilumab improved SWOV by 2.23-fold (nominal p=0.003; Table 16). Additional sensitivity analyses demonstrated the significant improvement of SWOV with lenzilumab treatment over and above any improvement afforded by the combined use of remdesivir plus steroids (n=331) by 1.92-fold (nominal p=0.0067; Table 16). SWOV in subjects treated with lenzilumab was significantly improved by 1.88-fold (nominal p=0.015) when randomization day was no more than the median of two days after hospitalization (Table 16).

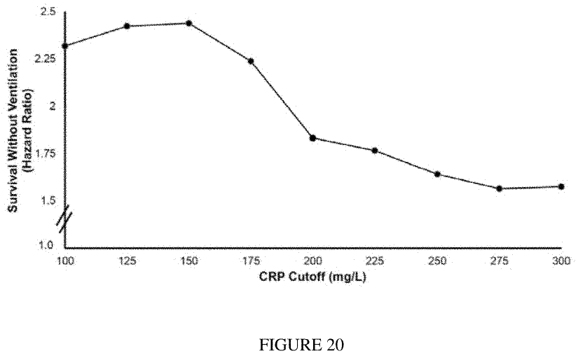

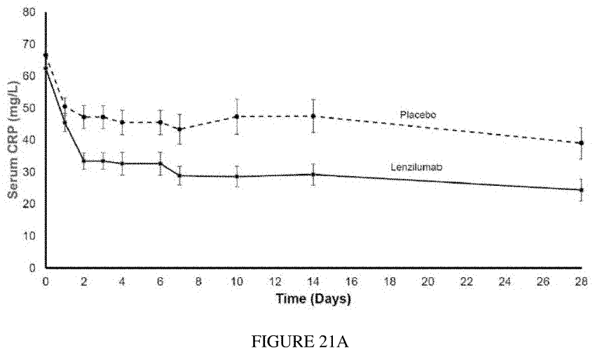

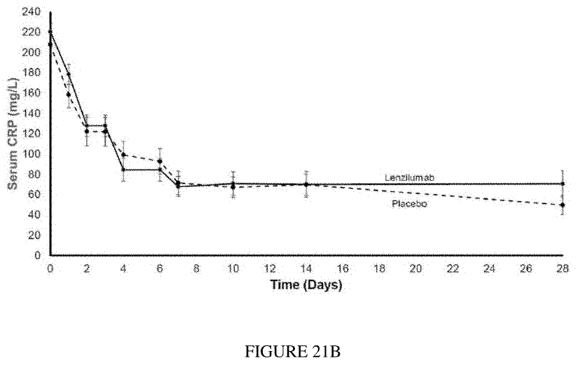

Further analysis of CRP<150 mg/L demonstrates a sigmoidal relationship where subjects with these characteristics receive the greatest likelihood of achieving SWOV (FIG. 20). Lenzilumab decreased CRP levels (FIG. 21A), beginning two days after administration and continuing for 28 days, but only in subjects with a baseline CRP<150 mg/L (FIG. 21B).

### Secondary Outcomes

Incidence of IMV, ECMO or death was 15.4% in the lenzilumab group and 21.4% in placebo (OR 0.67; 95% CI 0.41-1.10), p=ns) but was significantly improved in subjects with CRP<150 mg/L and age<85 (OR 0.32; 95% CI, 0.15-0.65, nominal p=0.002). (Table 17)

Mortality was significantly improved by 2.17-fold in subjects with CRP<150 mg/L and age<85 (nominal p=0.040). Similarly, Time to Recovery was not different between groups in the mITT population but was significantly improved by 36% (nominal p=0.012) in the mITT population with CRP<150 mg/L and age<85.

TABLE 17
Secondary Efficacy Outcomes, mITTₐ

| | | | Hazard Ratio | |
|---|---|---|---|---|
| | | | or Odds Ratio | |
| Outcome | Lenzilumab | Placebo | (95% CI) | P Value |
| Incidence IMV, | 15.4 | 21.4 | 0.67 | 0.111 |
| ECMO or | (11.1-21.0) | (16.3-27.4) | (0.41-1.10) | |
| death (%) | (n = 236) | (n = 243) | | |
| CRP <150 mg/L | 7.2 | 19.5 | 0.32 | 0.002 |
| Age <85 | (4.0-12.7) | (13.8-26.9) | (0.15-0.65) | |
| (n = 336) | (n = 157) | (n = 179) | | |
| Mortality (%) | 9.6 | 13.9 | 1.38 | 0.239 |
| | (6.4-14.2) | (10.1-19.0) | (0.81-2.37) | |
| | (n = 236) | (n = 243) | | |
| CRP <150 mg/L, | 6.6 | 13.8 | 2.17 | 0.040 |
| Age <85 | (3.6-11.9) | (9.5-19.9) | (1.04-4.54) | |
| (n = 336) | (n = 157) | (n = 179) | | |
| Time to Recovery | 8 | 8 | 1.09 | 0.43 |
| (median days) | (7-9) | (7-9) | (0.88-1.35) | |
| | (n = 236) | (n = 243) | | |
| CRP <150 mg/L, | 7 | 8 | 1.36 | 0.012 |
| Age <85 | (6-7) | (7-8) | (1.07-1.73) | |
| (n = 336) | (n = 157) | (n = 179) | | |

ₐAll data censored at 28 days following enrollment
^{b}Kaplan-Meier estimates for proportion of subjects. Value represent estimate (95% CI)
^{c}Cox Proportional Hazard Model for time to event.

### Safety

In the safety population, adverse events ≥Grade 3 were reported in 26.7% and 32.7% of the lenzilumab and placebo subjects, respectively (Table 18). Serious adverse events were also similar and reported in 24.7% and 29.6% of the subjects in the lenzilumab and placebo groups, respectively. Lenzilumab, compared with placebo, produced no infusion-related reactions, and no attributable serious adverse events; including, hematologic laboratory abnormalities, liver enzyme abnormalities, pulmonary alveolar proteinosis, or increased incidence of infection.

TABLE 18
Most Common Grade ≥3 Adverse Events
(Overall Incidence >1.0%)

| System Organ Class | | Lenzilumab | Placebo | Total |
|---|---|---|---|---|
| | Preferred Term n (%) | N = 255 | N = 257 | N = 512 |
| Any AE ≥ Grade 3 | | 68 (26.7) | 84 (32.7) | 152 (29.7) |
| Respiratory, thoracic and | | 64 (25.1) | 71 (27.6) | 135 (26.4) |
| mediastinal disorders | | | | |
| | Respiratory failure | 24 (9.4) | 31 (12.1) | [symbol] 5 (10.7) |
| | Acute respiratory failure | 18 (7.1) | 22 (8.6) | 40 (7.8) |
| | Hypoxia | 15 (5.9) | 15 (5.8) | 30 (5.9) |
| | Pulmonary embolism | 5 (2.0) | 3 (1.2) | 8 (1.6) |
| | Acute respiratory distress | 4 (1.6) | 3 (1.2) | 7 (1.4) |
| | syndrome | | | |
| Cardiac disorders | | 15 (5.9) | 14 (5.4) | 29 (5.7) |
| | Cardiac arrest | 8 (3.1) | 4 (1.6) | 12 (2.3) |
| | Cardio-respiratory arrest | 3 (1.2) | 4 (1.6) | 7 (1.4) |
| Infections and infestations | | 10 (3.9) | 16 (6.2) | 26 (5.1) |
| | Septic shock | 5 (2.0) | 9 (3.5) | 14 (2.7) |
| | Sepsis | 2 (0.8) | 5 (1.9) | 7 (1.4) |
| | Pneumonia bacterial | 0 (0.0) | 6 (2.3) | 6 (1.2) |
| Vascular disorders | | 10 (3.9) | 15 (5.8) | 25 (4.9) |
| | Shock | 3 (1.2) | 6 (2.3) | 9 (1.8) |
| | Hypotension | 2 (0.8) | 5 (1.9) | 7 (1.4) |
| Renal and urinary disorders | | 5 (2.0) | 11 (4.3) | 16 (3.1) |
| | Acute kidney injury | 5 (2.0) | 8 ( [symbol] .1) | 1 [symbol] (2.5) |
| General disorders and | | 4 (1.6) | 11 (4.3) | 15 (2.9) |
| administration site conditions | | | | |
| | Multiple organ dysfunction | 3 (1.2) | 6 (2.3) | 9 (1.8) |
| | syndrome | | | |

[symbol]

indicates data missing or illegible when filed

### Discussion

The clinical manifestations of coronavirus disease 2019 (COVID-19) range from asymptomatic disease to critical illness, acute respiratory disease (ARDS), and death. These pathologies result from the viral-induced hyperinflammatory immune response, or cytokine storm (CS). CS in COVID-19 is similar to that resulting from chimeric antigen receptor T-cell therapy (CAR-T), hemophagocytic lymphohistiocytosis (HLH), macrophage activation syndrome (MAS), and acute graft-versus-host disease (GvHD); where CS intensity is directly correlated with the severity of tissue injury, neurological manifestations, and adverse clinical outcomes. CS is characterized by granulocyte-macrophage colony-stimulating factor (GM-CSF)-mediated activation and trafficking of myeloid cells, leading to elevations of downstream inflammatory chemokines (MCP-1, IL-8, IP-10), cytokines (IL-6, IL-1), and other markers of systemic inflammation (CRP, D-dimer, ferritin).

In COVID-19, high levels of GM-CSF-secreting T-cells have been associated with disease severity, myeloid cell trafficking to the lungs, and intensive care unit (ICU) admission. Elevation of circulating GM-CSF in subjects with emerging hyperinflammation four days after symptom onset differentiated mild/moderate from severe disease. Since GM-CSF is produced by activated T-cells in tissue microenvironments and is bound by extracellular matrix and receptors, its detection in the serum indicates significantly elevated tissue levels that orchestrate local and subsequently, systemic hyperinflammatory activity.

CRP levels greater than 100 mg/L upon hospital admission are one of the strongest predictors of critical illness, even more so than age greater than 75 years or other comorbid conditions associated with COVID-19. Subjects with CRP above 150 mg/L are defined as experiencing COVID-19-associated hyperinflammation (COV-HI) and are at risk of escalated respiratory support or death. CRP is a readily available and cost effective biomarker that can be used to delineate the state of COV-HI, help to identify subjects with emerging CS, and predict the need for early intervention to prevent or reduce progression to IMV and intensive care.

Lenzilumab is a novel Humaneered® anti-human GM-CSF monoclonal antibody that directly binds GM-CSF and prevents signaling through its receptor. It has high binding affinity (25 pM) for glycosylated human GM-CSF and a slow off-rate. Lenzilumab has more than 94% specificity to the human germline to minimize immunogenicity, is highly soluble at a pH of 7.0 to minimize potential for infusion reactions, and in an extensive pre-clinical toxicology program demonstrates a No Observed Adverse Effect Level (NOAEL) of >100 mg/kg/week (data not shown). No treatment emergent serious adverse events (SAEs) or Suspected Unexpected Serious Adverse Reactions (SUSARSs) have been reported across any of the four completed clinical development studies. Lenzilumab can be dosed in a regimen designed to achieve therapeutic concentrations to neutralize GM-CSF in both serum and lung tissue. In a matched case cohort study of high-risk COVID-19 subjects with severe pneumonia; lenzilumab was associated with a significantly shorter time to clinical improvement, defined as at least a 2-point improvement in an 8-point ordinal scale; and an incidence of invasive mechanical ventilation (IMV) or death of 8% compared with 40% (p=0.07) in control patients. (Example 10)

The LIVE-AIR trial in newly hospitalized COVID-19 patients, who were hypoxic but had not progressed to IMV showed that early lenzilumab treatment improved the relative likelihood of SWOV through Day 28 by 54%. This finding was supported by sensitivity analyses in the ITT population. Further sensitivity analyses showed that the improvement in likelihood of SWOV was over and above treatment with remdesivir and corticosteroids. Additionally, improvement in SWOV was observed in subjects hospitalized for less than 2 days. Finally, improvement in SWOV was most evident in patients with CRP<150 mg/L who are less than 85 years of age. Lenzilumab was associated with similar improvements on key secondary endpoints including; incidence of IMV, ECMO or death, mortality, and time to recovery in those subjects with CRP<150 mg/L and <85 years. Treatment with lenzilumab was safe and well tolerated with a serious adverse event profile no different from placebo.

Safe and effective therapeutics to treat hospitalized subjects with COVID-19 remains a significant unmet clinical need. The NIH currently recommends against the use of dexamethasone in hospitalized, hypoxic subjects who do not require supplemental oxygen and data regarding the benefit of remdesivir in this population are conflicting and insufficient to recommend for use. Recommendations supporting the combination of dexamethasone and remdesivir in hospitalized subjects on low-flow supplemental oxygen are based primarily on expert opinion according to the NIH as no randomized controlled studies have evaluated these agents in combination. The primary population where the use of dexamethasone has proven to significantly improve mortality is in subjects on IMV who are <70 years of age. Data reported on the use of tocilizumab is conflicting; and baricitinib is reserved for those with contraindications to corticosteroid. In LIVEAIR, early intervention with lenzilumab improved the relative likelihood of SWOV through Day 28 by 54% in hospitalized, racially and ethnically diverse subjects with COVID-19, who had several comorbid conditions and were hypoxic but did not require mechanical ventilation at baseline.

CRP was analyzed as it is a marker of systemic inflammation, routinely used clinically and known to be proportionally elevated with the severity of COVID-19 disease. In this trial, 77% of subjects with an evaluable CRP, had a baseline value <150 mg/L and 74% had a baseline CRP value <150 mg/L and age<85. The CRP analysis clearly demonstrated that CRP less than 150 mg/L was associated with best likelihood of achieving SWOV and clearly differentiated emerging hyperinflammation from full-blown CS. In subjects with CRP<150 mg/L and <85 years old, lenzilumab improved SWOV by nearly 3-fold and was accompanied by a 2.17-fold improvement in mortality as well as a 36% improvement in time to recovery. Given that CRP is a readily available, routinely measured biomarker of systemic inflammation, with prognostic value affirmed in this and other trials, it can clearly be used to clinically identify a phenotype of COVID-19 disease that best responds to lenzilumab treatment.

LIVE-AIR used a composite primary endpoint of SWOV which includes time to death or time to invasive mechanical ventilation. This endpoint has been called “ventilator-free survival” and “alive and ventilator-free”. The nomenclature selected in LIVE-AIR more accurately captures the meaning of the endpoint without confusion by alternate interpretations. Regardless of nomenclature, the composite endpoint is appropriate for an event-based clinical trial of this size to predict a clinically meaningful outcome that equates ventilation with mortality. It is not con-founded by mechanisms that may preferentially affect either ventilation or death. Lenzilumab's ability to improve SWOV was more pronounced in combination with current treatments including remdesivir which has demonstrated a 5 day improvement in time to recovery and dexamethasone which improved survival in subjects needing respiratory support, which may indicate a synergistic mechanism of action with the triple combination. This robust improvement in the composite endpoint demonstrates the potential for lenzilumab treatment to result in a true improvement in survival since nearly all subjects that die from COVID-19 have received IMV during their hospital stay. Confirmation of the lenzilumab improvement of survival, and the role of SWOV as a surrogate endpoint, could be confirmed in a large adequately powered mortality trial.

The results with lenzilumab on SWOV demonstrate that impeding GM-CSF signaling early in CS is required to prevent COVID-19 disease progression to IMV or death. This is further supported by an 88% improvement in SWOV in subjects hospitalized <2 days. The results herein with lenzilumab confirm results from prior clinical trials targeting GM-CSF but are differentiated by patient selection, timing of intervention, and dosing strategy. Mavrilimumab, a monoclonal antibody that acts as a GM-CSF alpha-receptor antagonist demonstrated non-significant trends of 48% improvement in survival without requirement for supplemental oxygen. Otilimab, a monoclonal antibody targeting circulating GM-CSF in ventilated subjects, was reported in a non-peer-reviewed disclosure of results from its phase 2b COVID-19 study (NCT04376684) to not meet its primary endpoint of improvement in the proportion of COVID-19 subjects who were alive and free of respiratory failure 28 days; however, in pre-planned analysis of subjects aged at least 70 years, which comprised 22% of the entire patient population, 65.1% were alive and free of respiratory failure, compared to 45.9% of subjects who received the standard of care. The results of all three trials of GM-CSF antagonism suggest the central role of GM-CSF in CS of COVID-19 that has been recently demonstrated through network analysis of cytokine profiles from COVID-19 subjects. This analysis associated IL-6, CXCL10, but in particular GM-CSF, to markers of endothelial injury and thrombosis early in COVID-19, in severe cases, in hospitalized subjects and those with risk factors for severe disease, and uniquely elevated in fatal COVID-19. The results herein with lenzilumab more completely define the central role of GM-CSF in COVID-19, identified subjects more likely to receive benefit from lenzilumab and underscores the necessity to target GM-CSF early during the emerging hyperinflammatory stage in order to ameliorate CS induced by COVID-19.

The findings of the network analysis and the anti-GM-CSF approaches listed above, including the robust response to lenzilumab particularly in subjects with CRP levels <150 mg/L and age<85, helps explain the limited efficacy of other anti-cytokine approaches that target cytokines downstream of myeloid cell activation in the CS cascade (i.e., IL-6, TNFα, IL-10). Results from clinical trials with anti-IL-6 agents are mixed and mostly negative. Targeting single downstream mediators of CS, such as IL-6, may render therapy clinically ineffective as other key, redundant cytokines continue to promote tissue damage or are even compensatory upregulated in the absence of IL-6 signaling. Alternatively, it is possible that targeting IL-6 is more effective later in the disease course as evidenced by tocilizumab wherein benefit appears to be derived in more critically ill subjects recently admitted to the ICU.

In the LIVE-AIR study, lenzilumab was studied as a real-world early intervention in hospitalized, hypoxic subjects not requiring invasive mechanical ventilation; hypothesizing that neutralizing GM-CSF during emerging hyperinflammation, could prevent the consequences of full-blown CS, in subjects at risk but already treated with standard of care. Lenzilumab improved the robust endpoint of SWOV, the effect of which was more pronounced in emerging hyperinflammation than in subjects who were further evolved in their disease course. LIVE-AIR has two important limitations. First, while it demonstrated an improvement in survival in subjects with CRP<150 mg/L and aged <85, the study was not powered to observe a survival benefit, and indeed did not achieve statistical significance on this key secondary endpoint in the mITT population. Second, the observations pertaining to benefits in the population that had CRP<150 mg/L and age <85 were exploratory.

Despite high vaccine efficacy and uptake in some countries, hospitalizations for COVID will continue to occur given the propensity for mutation and escape from protection that occurs through natural immunological selection. A proportion of those subjects will unfortunately progress to hypoxia and hospitalization. For them, dexamethasone is currently the only treatment approach shown to provide a survival benefit. This study demonstrates that early neutralization of GM-CSF with lenzilumab, a key initiator and orchestrator of CS, in newly hospitalized hypoxic subjects can improve their likelihood of survival without the need for mechanical ventilation and defines a targeted patient population most likely to derive the greatest benefit over and above that of steroids and/or remdesivir.

1. A method for treating a subject having emergent COVID-19-associated hyperinflammation (COV-HI), the method comprising administering to the subject a therapeutically effective amount of a pharmaceutical composition comprising an hGM-CSF antagonist.

2. The method of claim 1, wherein the hGM-CSF antagonist is anti-hGM-CSF antibody Lenzilumab.

3. The method of claim 1, wherein the hGM-CSF antagonist is an anti-GM-SCF antibody selected from the group consisting of Namilumab, Otilimab, Gimsilumab, and TJM2 (TJ003234).

4. The method of claim 1, wherein the GM-CSF antagonist is anti-GM-CSF receptor antibody Mavrilimumab.

5. The method of claim 2, wherein the subject has a baseline serum level of C-reactive protein (CRP) of from greater than 5 mg/L, to less than 150 mg/L before administration of the hGM-CSF antagonist.

6. The method of claim 3, wherein the subject has a baseline serum level of C-reactive protein (CRP) of from greater than 5 mg/L to less than 150 mg/L before administration of the hGM-CSF antagonist.

7. The method of claim 4, wherein the subject has a baseline serum level of C-reactive protein (CRP) of from greater than 5 mg/L, to less than 150 mg/L before administration of the hGM-CSF antagonist.

8. The method of claim 2, wherein the subject is from 18 years old to less than 85 years old.

9. The method of claim 3, wherein the subject is from 18 years old to less than 85 years old.

10. The method of claim 4, wherein the subject is from 18 years old to less than 85 years old.

11. The method of claim 2, wherein the subject has a baseline serum level of C-reactive protein (CRP) of from greater than 5 mg/L to less than 150 mg/L before administration of the hGM-CSF antagonist and is from 18 years old to less than 85 years old.

12. The method of claim 3, wherein the subject has a baseline serum level of C-reactive protein (CRP) of from greater than 5 mg/L to less than 150 mg/L before administration of the hGM-CSF antagonist and is from 18 years old to less than 85 years old.

13. The method of claim 4, wherein the subject has a baseline serum level of C-reactive protein (CRP) of from greater than 5 mg/L to less than 150 mg/L before administration of the hGM-CSF antagonist and is from 18 years old to less than 85 years old.

14. The method of claim 2, wherein the subject has a baseline serum level of GM-CSF of three or more times higher than 10 pg per milliliter, serum ferritin level >300 ug/L and serum D-dimer level of 500 nanograms per milliliter (mL) or higher.

15. The method of claim 3, wherein the subject has a baseline serum level of GM-CSF of three or more times higher than 10 pg per milliliter, serum ferritin level >300 ug/L and serum D-dimer level of 500 nanograms per milliliter (mL) or higher.

16. The method of claim 4, wherein the subject has a baseline serum level of GM-CSF of three or more times higher than 10 pg per milliliter, serum ferritin level >300 ug/L and serum D-dimer level of 500 nanograms per milliliter (mL) or higher.

17. The method of claim 2, wherein the subject has severe COVID-19 pneumonia and/or has a pulse oximeter reading (SpO2)≤94% on room air and/or requires supplemental oxygen.

18. The method of claim 3, wherein the subject has severe COVID-19 pneumonia and/or has a pulse oximeter reading (SpO2)≤94% on room air and/or requires supplemental oxygen.

19. The method of claim 4, wherein the subject has severe COVID-19 pneumonia and/or has a pulse oximeter reading (SpO2)≤94% on room air and/or requires supplemental oxygen.

20. The method of claim 17, further comprising administering supplemental oxygen to the subject, wherein the supplemental oxygen is low-flow oxygen, high-flow oxygen, or non-invasive positive pressure ventilation (NPPV).

21. The method of claim 18, further comprising administering supplemental oxygen to the subject, wherein the supplemental oxygen is low-flow oxygen, high-flow oxygen, or non-invasive positive pressure ventilation (NPPV).

22. The method of claim 19, further comprising administering supplemental oxygen to the subject, wherein the supplemental oxygen is low-flow oxygen, high-flow oxygen, or non-invasive positive pressure ventilation (NPPV).

23. The method of claim 20, wherein the subject does not require administration of supplemental oxygen by invasive mechanical ventilator (IMV).

24. The method of claim 21, wherein the subject does not require administration of supplemental oxygen by invasive mechanical ventilator (IMV).

25. The method of claim 22, wherein the subject does not require administration of supplemental oxygen by invasive mechanical ventilator (IMV).

26. The method of claim 2, further comprising administering antiviral agent remdesivir and/or a combination of remdesivir and a steroid.

27. The method of claim 3, further comprising administering antiviral agent remdesivir and/or a combination of remdesivir and a steroid.

28. The method of claim 4, further comprising administering antiviral agent remdesivir and/or a combination of remdesivir and a steroid.

29. The method of claim 2, wherein the hGM-CSF antagonist is administered within one to two days of hospitalization of the subject.

30. The method of claim 3, wherein the hGM-CSF antagonist is administered within one to two days of hospitalization of the subject.

31. The method of claim 4, wherein the hGM-CSF antagonist is administered within one to two days of hospitalization of the subject.

32. The method of claim 2, wherein the administration of the hGM-CSF antagonist improves survival without ventilation (SWOV) of the subject up to at least 28 days after the administration, improvement of SWOV is an improvement compared to an improvement in SWOV of a subject treated with a placebo.

33. The method of claim 3, wherein the administration of the hGM-CSF antagonist improves survival without ventilation (SWOV) of the subject up to at least 28 days after the administration, improvement of SWOV is an improvement compared to an improvement in SWOV of a subject treated with a placebo.

34. The method of claim 4, wherein the administration of the hGM-CSF antagonist improves survival without ventilation (SWOV) of the subject up to at least 28 days after the administration, improvement of SWOV is an improvement compared to an improvement in SWOV of a subject treated with a placebo.

35. The method of claim 2, wherein the hGM-CSF antagonist is administered at a dose of from 1200 mg to 1800 mg over 24 hours.

36. The method of claim 3, wherein the hGM-CSF antagonist is administered at a dose of from 1200 mg to 1800 mg over 24 hours.

37. The method of claim 4, wherein the hGM-CSF antagonist is administered at a dose of from 1200 mg to 1800 mg over 24 hours.

38. The method of claim 35, wherein the administered dose is 1,104 mg to 1,656 mg over 24 hours.

39. The method of claim 36, wherein the administered dose is 1,104 mg to 1,656 mg over 24 hours.

40. The method of claim 37, wherein the administered dose is 1,104 mg to 1,656 mg over 24 hours.

41. The method of claim 35, wherein the administered dose is 552 mg every eight hours

42. The method of claim 36, wherein the administered dose is 552 mg every eight hours

43. The method of claim 37, wherein the administered dose is 552 mg every eight hours

44. A method for treating a subject hospitalized with severe COVID-19 pneumonia and having a baseline level of C-reactive protein (CRP) of less than 150 mg/L, the method comprising administering to the subject a therapeutically effective amount of a pharmaceutical composition comprising an hGM-CSF antagonist.

45. The method of claim 44, wherein the hGM-CSF antagonist is anti-hGM-CSF antibody Lenzilumab.

46. The method of claim 44, wherein the GM-CSF antagonist is an anti-GM-SCF antibody selected from the group consisting of Namilumab, Otilimab, Gimsilumab, and TJM2 (TJ003234).

47. The method of claim 44, wherein the GM-CSF antagonist is anti-GM-CSF receptor antibody Mavrilimumab.

48. The method of claim 45, wherein the subject has a baseline serum level of C-reactive protein (CRP) of from greater than 5 mg/L to less than 150 mg/L before administration of the hGM-CSF antagonist.

49. The method of claim 46, wherein the subject has a baseline serum level of C-reactive protein (CRP) of from greater than 5 mg/L to less than 150 mg/L before administration of the hGM-CSF antagonist.

50. The method of claim 47, wherein the subject has a baseline serum level of C-reactive protein (CRP) of from greater than 5 mg/L to less than 150 mg/L before administration of the hGM-CSF antagonist.

51. The method of claim 45, wherein the subject is from 18 years old to less than 85 years old.

52. The method of claim 46, wherein the subject is from 18 years old to less than 85 years old.

53. The method of claim 47, wherein the subject is from 18 years old to less than 85 years old.

54. The method of claim 45, wherein the subject has a baseline serum level of C-reactive protein (CRP) of from greater than 5 mg/L to less than 150 mg/L before administration of the hGM-CSF antagonist and is from 18 years old to less than 85 years old.

55. The method of claim 46, wherein the subject has a baseline serum level of C-reactive protein (CRP) of from greater than 5 mg/L to less than 150 mg/L before administration of the hGM-CSF antagonist and is from 18 years old to less than 85 years old.

56. The method of claim 47, wherein the subject has a baseline serum level of C-reactive protein (CRP) of from greater than 5 mg/L to less than 150 mg/L before administration of the hGM-CSF antagonist and is from 18 years old to less than 85 years old.

57. The method of claim 45, wherein the subject has a baseline serum level of GM-CSF of three or more times higher than 10 pg per milliliter, serum ferritin level >300 ug/L and serum D-dimer level of 500 nanograms per milliliter (mL) or higher.

58. The method of claim 46, wherein the subject has a baseline serum level of GM-CSF of three or more times higher than 10 pg per milliliter, serum ferritin level >300 ug/L and serum D-dimer level of 500 nanograms per milliliter (mL) or higher.

59. The method of claim 47, wherein the subject has a baseline serum level of GM-CSF of three or more times higher than 10 pg per milliliter, serum ferritin level >300 ug/L and serum D-dimer level of 500 nanograms per milliliter (mL) or higher.

60. The method of claim 45, wherein the subject has a pulse oximeter reading (SpO2)≤94% on room air and/or requires supplemental oxygen.

61. The method of claim 46, wherein the subject has a pulse oximeter reading (SpO2)≤94% on room air and/or requires supplemental oxygen.

62. The method of claim 47, wherein the subject has a pulse oximeter reading (SpO2)≤94% on room air and/or requires supplemental oxygen.

63. The method of claim 60, further comprising administering supplemental oxygen to the subject, wherein the supplemental oxygen is low-flow oxygen, high-flow oxygen, or non-invasive positive pressure ventilation (NPPV).

64. The method of claim 61, further comprising administering supplemental oxygen to the subject, wherein the supplemental oxygen is low-flow oxygen, high-flow oxygen, or non-invasive positive pressure ventilation (NPPV).

65. The method of claim 62, further comprising administering supplemental oxygen to the subject, wherein the supplemental oxygen is low-flow oxygen, high-flow oxygen, or non-invasive positive pressure ventilation (NPPV).

66. The method of claim 63, wherein the subject does not require administration of supplemental oxygen by invasive mechanical ventilator (IMV).

67. The method of claim 64, wherein the subject does not require administration of supplemental oxygen by invasive mechanical ventilator (IMV).

68. The method of claim 65, wherein the subject does not require administration of supplemental oxygen by invasive mechanical ventilator (IMV).

69. The method of claim 45, further comprising administering antiviral agent remdesivir and/or a combination of remdesivir and a steroid.

70. The method of claim 46, further comprising administering antiviral agent remdesivir and/or a combination of remdesivir and a steroid.

71. The method of claim 47, further comprising administering antiviral agent remdesivir and/or a combination of remdesivir and a steroid.

72. The method of claim 45, wherein the hGM-CSF antagonist is administered within one to two days of hospitalization of the subject.

73. The method of claim 46, wherein the hGM-CSF antagonist is administered within one to two days of hospitalization of the subject.

74. The method of claim 47, wherein the hGM-CSF antagonist is administered within one to two days of hospitalization of the subject.

75. The method of claim 45, wherein the administration of the hGM-CSF antagonist improves survival without ventilation (SWOV) of the subject up to at least 28 days after the administration, wherein improvement of SWOV is an improvement compared to an improvement in SWOV of a subject treated with a placebo.

76. The method of claim 46, wherein the administration of the hGM-CSF antagonist improves survival without ventilation (SWOV) of the subject up to at least 28 days after the administration, wherein improvement of SWOV is an improvement compared to an improvement in SWOV of a subject treated with a placebo.

77. The method of claim 47, wherein the administration of the hGM-CSF antagonist improves survival without ventilation (SWOV) of the subject up to at least 28 days after the administration, wherein improvement of SWOV is an improvement compared to an improvement in SWOV of a subject treated with a placebo.

78. The method of claim 45, wherein the hGM-CSF antagonist is administered at a dose of from 1200 mg to 1800 mg over 24 hours.

79. The method of claim 46, wherein the hGM-CSF antagonist is administered at a dose of from 1200 mg to 1800 mg over 24 hours.

80. The method of claim 47, wherein the hGM-CSF antagonist is administered at a dose of from 1200 mg to 1800 mg over 24 hours.

81. The method of claim 78, wherein the administered dose is 1,104 mg to 1,656 mg over 24 hours.

82. The method of claim 79, wherein the administered dose is 1,104 mg to 1,656 mg over 24 hours.

83. The method of claim 80, wherein the administered dose is 1,104 mg to 1,656 mg over 24 hours.

84. The method of claim 78, wherein the administered dose is 552 mg every eight hours.

85. The method of claim 79, wherein the administered dose is 552 mg every eight hours.

86. The method of claim 80, wherein the administered dose is 552 mg every eight hours.
